(19) United States
(12) Patent Application Publication
(10) Pub. No.: US 2022/0380432 A1
(43) Pub. Date: Dec. 1, 2022

(54) Acylated Calcitonin Mimetics

(71) Applicant: KeyBioscience AG, Stans (CH)

(72) Inventors: Kim V. Andreassen, Frederiksberg (DK); Kim Henriksen, Hillerød (DK); Nina Sonne, Kobenhavn K (DK); Morten Asser Karsdal, Kobenhavn (DK)

(73) Assignee: KeyBioscience AG, Stans (CH)

(21) Appl. No.: 17/270,331

(22) PCT Filed: Aug. 22, 2019
(86) PCT No.: PCT/EP2019/072533

§ 371 Date: Feb. 22, 2021

(30) Foreign Application Priority Data
Aug. 22, 2018 (GB) 1813678.8

(51) Int. Cl. C07K 14/585; A61K 47/54

(52) U.S. Cl. CPC C07K 14/585; A61K 38/00; A61K 47/542

(57) ABSTRACT

Disclosed herein are calcitonin mimetics that are acylated at a lysine residue located at the 11 position or 19 position of the calcitonin mimetic, and the use thereof as medicaments in the treatment of various diseases and disorders, including diabetes, excess bodyweight, excessive food consumption and metabolic syndrome, NASH, alcoholic and non-alcoholic fatty liver disease, the regulation of blood glucose levels, the regulation of response to glucose tolerance tests, the regulation of food intake, and the treatment of osteoporosis and the treatment of osteoarthritis.

The present invention relates to acylated mimetics of calcitonin, and extends to their use as medicaments in the treatment of various diseases and disorders, including, but not limited to diabetes (Type I and Type II), excess bodyweight, excessive food consumption and metabolic syndrome, non-alcoholic steatohepatitis (NASH), alcoholic and non-alcoholic fatty liver disease, the regulation of blood glucose levels, the regulation of response to glucose tolerance tests, the regulation of food intake, the treatment of osteoporosis and the treatment of osteoarthritis.

Worldwide, there are about 250 million diabetics and the number is projected to double in the next two decades. Over 90% of this population suffers from type 2 diabetes mellitus (T2DM). It is estimated that only 50-60% of persons affected with T2DM or in stages preceding overt T2DM are currently diagnosed.

T2DM is a heterogeneous disease characterized by abnormalities in carbohydrate and fat metabolism. The causes of T2DM are multi-factorial and include both genetic and environmental elements that affect β-cell function and insulin sensitivity in tissues such as muscle, liver, pancreas and adipose tissue. As a consequence impaired insulin secretion is observed and paralleled by a progressive decline in β-cell function and chronic insulin resistance. The inability of the endocrine pancreas to compensate for peripheral insulin resistance leads to hyperglycaemia and onset of clinical diabetes. Tissue resistance to insulin-mediated glucose uptake is now recognized as a major pathophysiologic determinant of T2DM.

A success criterion for an optimal T2DM intervention is the lowering of blood glucose levels, which can be both chronic lowering of blood glucose levels and increased ability to tolerate high glucose levels after food intake, described by lower peak glucose levels and faster clearance. Both of these situations exert less strain on β-cell insulin output and function.

Type I diabetes is characterised by a loss of the ability to produce insulin in response to food intake and hence an inability to regulate blood glucose to a normal physiological level.

The physical structure of bone may be compromised by a variety of factors, including disease and injury. One of the most common bone diseases is osteoporosis, which is characterized by low bone mass and structural deterioration of bone tissue, leading to bone fragility and an increased susceptibility to fractures, particularly of the hip, spine and wrist. Osteoporosis develops when there is an imbalance such that the rate of bone resorption exceeds the rate of bone formation. Administering an effective amount of an anti-resorptive agent, such as calcitonin, has shown to prevent resorption of bone.

Inflammatory or degenerative diseases, including diseases of the joints, e.g. osteoarthritis (OA), rheumatoid arthritis (RA) or juvenile rheumatoid arthritis (JRA), and including inflammation that results from autoimmune response, e.g. lupus, ankylosing spondylitis (AS) or multiple sclerosis (MS), can lead to substantial loss of mobility due to pain and joint destruction. Cartilage that covers and cushions bone within joints may become degraded over time thus undesirably permitting direct contact of two bones that can limit motion of one bone relative to the other and/or cause damage to one by the other during motion of the joint. Subchondral bone just beneath the cartilage may also degrade. Administering an effective amount of an anti-resorptive agent, such as calcitonin, may prevent resorption of bone.

Calcitonins are highly conserved over a wide range of species. Full-length native calcitonin is 32 amino acids in length. The sequences of examples of natural calcitonins are set out below:

Salmon
CSNLSTCVLGKLSQELHKLQTYPRTNTGSGTP
Eel
CSNLSTCVLGKLSQELHKLQTYPRTDVGAGTP
Chicken
CASLSTCVLGKLSQELHKLQTYPRTDVGAGTP
Mouse
CGNLSTCMLGTYTQDLNKFHTFPQTSIGVEAP
Rat
CGNLSTCMLGTYTQDLNKFHTFPQTSIGVGAP
Horse
CSNLSTCVLGTYTQDLNKFHTFPQTAIGVGAP
Canine-1
CSNLSTCVLGTYSKDLNNFHTFSGIGFGAETP
Canine-2
CSNLSTCVLGTYTQDLNKFHTFPQTAIGVGAP
Porcine
CSNLSTCVLSAYWRNLNNFHRFSGMGFGPETP
Human
CGNLSTCMLGTYTQDFNKFHTFPQTAIGVGAP

Synthetic variants of natural calcitonins having modified amino acid sequences which are intended to provide improved properties are disclosed in WO2013/067357 and WO 2015/071229.

However, peptides, such as calcitonin and calcitonin mimetics, typically have poor absorption, distribution, metabolism and excretion properties, with rapid clearance and short half-life. Accordingly, peptide drugs typically require daily parenteral administration. Daily administration of treatment through subcutaneous (s.c.) injections is currently not an optimal method of administration, as it poses as an inconvenience to individual patients, and may cause non-adherence to treatment plan to avoid the discomfort associated with daily injections. As such, a once weekly drug using s.c. injections would increase the quality of life for the patients in question and further assist to adherence of treatment plan.

There are numerous approaches known in the art for attempting to improve the in-vivo half-life of peptide drugs. Such approaches include improving proteolytic stability (by, e.g., protecting the N- and C-termini, replacing amino acids with D-amino acids or unnatural amino acids, cyclising the peptide, etc.) and reducing renal clearance (by, e.g., conjugating the peptide to macromolecules, such as large polymers, albumin, immunoglobulins, etc.). However, it is also known in the art that making such modifications to drug peptides can be deleterious in terms of, for example, reduced drug potency and unpredictable adverse side reactions, such as drug sensitisation. As such, it is not possible to predict whether such modifications necessarily would improve the therapeutic profile of a peptide drug.

Accordingly, developing peptide drugs that require only once-weekly administration is a challenging prospect.

One approach to improving the pharmacokinetic and pharmacodynamic properties of peptide drugs is to acylate the peptide. Trier et al (PhD thesis, 2016, “Acylation of Therapeutic Peptides”, DTU; available for download from http://orbit.dtu.dk/files/127682557/PhD_thesis_Sofie_Trier.pdf) studied the effect of acylating two therapeutic peptides, namely glucagon-like peptide 2 (GLP2) and salmon calcitonin (sCT), with acyl groups of varying length (C₈-C₁₆). Whilst the effects of acylating GLP2 were found to be largely predictable based on previous observations on similar peptides, the effects observed when acylating sCT were found to be unpredictable. For example, Trier et al found that acylating sCT (at various positions on the peptide backbone) consistently caused a substantial loss in receptor potency (60-80% loss), whereas receptor potency was retained for GLP-2 following acylation. Accordingly, whilst Trier et al. did uncover some useful properties associated with acylating sCT (particularly with regard to short chain (C₈) acylations), it was also clear that there were numerous unpredictable and significantly disadvantageous effects associated with acylating sCT, most notably a significant loss in receptor potency. An additional noteworthy point is that the studies of Trier et al. focused on acylating the 18 position (Lys18) of salmon calcitonin. This is because previous studies aiming at improving the efficacy of salmon calcitonin identified the 18 position as being the superior position for modification (in that instance by PEGylation, not acylation). In those studies it was found that PEGylating the Lys18 position of sCT resulted in better efficacy than the analogous peptides modified at the Cys1 or Lys11 positions (Youn et al, J. Control. Release, 2006, 334-342).

### SUMMARY OF THE INVENTION

The present inventors have found that acylating calcitonin mimetics at a lysine residue located at the 11 position of the calcitonin mimetics or at a lysine residue located at the 19 position of the calcitonin mimetics, in particular with certain specific acyl moieties, results in a surprising improvement in the efficacy of the peptide vis-à-vis the equivalent non-acylated peptide, as well as increasing the duration of action of the peptide. Similarly, it was found that the greatest improvement in efficacy of the calcitonin mimetic corresponded to acylation at the 11 or 19 position, whereas acylating the 18 position produced an inferior result, contrary to the findings in Youn et al. As such, the present inventors have developed potent novel acylated calcitonin mimetics that may only need to be administered once weekly, rather than once daily.

Accordingly, in one aspect, the present invention provides a calcitonin mimetic that is acylated at a lysine residue located at the 11 position of the calcitonin mimetic and/or that is acylated at a lysine residue located at the 19 position of the calcitonin mimetic. The side chain ε-amino group of said lysine residue is acylated with an acyl group selected from any one of the following: a C16 or longer fatty acid with an optional linker; or a C16 or longer fatty diacid with an optional linker.

As used herein, “calcitonin mimetic” means a peptide that activates the calcitonin receptor (i.e. a calcitonin receptor agonist), and preferably also activates the amylin receptor (i.e. a dual amylin and calcitonin receptor agonist).

In certain preferred embodiments, the calcitonin mimetic is from 32 to 37 amino acids in length. Most preferably the calcitonin mimetic is 32 amino acids in length. In one preferred aspect, in which the calcitonin mimetic is acylated at a lysine residue located at the 11 position, the present invention relates to a calcitonin mimetic of formula (I)(a):

CX₂X₃LSTCX₈LGK_{Ac}

wherein

X₂=A, G or S

X₃=N or S

X₈=M, V or α-aminoisobutyric acid (AiB)

and wherein K_{Ac} is a lysine residue wherein the side chain ε-amino group is acylated with an acyl group selected from any one of the following:

C₁₆ or longer fatty acid with an optional linker, or

C₁₆ or longer fatty diacid with an optional linker.

In another preferred aspect, in which the calcitonin mimetic is acylated at a lysine residue located at the 19 position, the present inventive relates to a calcitonin mimetic of formula (I)(b):

CX₂X₃LSTCX₈LGX₁₁X₁₂X₁₃X₁₄X₁₅X₁₆X₁₇X₁₈K_{Ac}

wherein

X₂=A, G or S

X₃=N or S

X₈=M, V or α-aminoisobutyric acid (AiB)

X₁₁=R, K, T, A or K_{Ac} (preferably R, K, or K_{Ac}, most preferably R or K)

X₁₂=L or Y (most preferably L)

X₁₃=S, T, W or Y (preferably T, S or Y)

X₁₄=Q, K, R or A (preferably Q or A, most preferably Q)

X₁₅=D, E or N (preferably D or E)

X₁₆=L or F (most preferably L)

X₁₇=H or N

X₁₈=R, K or N (preferably R or K)
and wherein K_{Ac} is a lysine residue wherein the side chain ε-amino group is acylated with an acyl group selected from any one of the following: C₁₆ or longer fatty acid with an optional linker, or

C₁₆ or longer fatty diacid with an optional linker.

Preferably, the calcitonin mimetic of formula (I)(a) or (I)(b) is from 32 to 37 amino acids in length, preferably 32, 33, 35, 36 or 37 amino acids in length. Most preferably, the calcitonin mimetic of formula (I)(a) or (I)(b) is 32 amino acids in length.

In a preferred aspect of the invention, the calcitonin mimetic is a 32mer calcitonin mimetic of formula (II):

CX₂X₃LSTCXLGX₁₁X₁₂X₁₃X₁₄X₁₅X₁₆X₁₇X₁₈X₁₉X₂₀X₂₁X₂₂X₂₃X₂₄X₂₅X₂₆X₂₇GX₂₉X₃₀X₃₁P

wherein

X₂=A, G or S

X₃=N or S

X₈=M, V or α-aminoisobutyric acid (AiB)

X₁₁=K_{Ac}, R, K, T or A (most preferably K_{Ac}, R or K)

X₁₂=L or Y

X₁₃=S, T, W or Y

X₁₄=Q, K, R or A

X₁₅=D, E or N

X₁₆=L or F

X₁₇=H or N

X₁₈=R, K or N

X₁₉=K_{Ac}, L, F or K (most preferably K_{Ac}, L or F)

X₂₀=Q, H or A

X₂₁=T or R

X₂₂=Y or F

X₂₃=S or P

X₂₄=G, K, Q or R

X₂₅=T, I or M

X₂₆=S, N, D, G or A

X₂₇=T, V, F or I

X₂₉=S, A, P or V

X₃₀=N, G or E

X₃₁=A, T or S (most preferably A or T)

wherein either X₁₁ is K_{Ac} and/or X₁₉ is K_{Ac} (such that either X₁₁ is K_{Ac} and X₁₉ is L, F or K, preferably L or F; or X₁₁ is R, K, T or A, preferably R or K, and X₁₉ is K_{Ac}; or X₁₁ is K_{Ac} and X₁₉ is K_{Ac}),

and wherein K_{Ac} is a lysine residue wherein the side chain ε-amino group is acylated with an acyl group selected from any one of the following:

C₁₆ or longer fatty acid,

C₁₆ or longer fatty diacid,

linker-C₁₆ or longer fatty acid, or

linker-C₁₆ or longer fatty diacid.

Preferably, the 32mer calcitonin mimetic of formula (II) is:

CX₂X₃LSTCX₈LGX₁₁ LX₁₃X₁₄X₁₅LX₁₇X₁₈X₁₉X₂₀TX₂₂PX₂₄TDVGANAP

wherein

X₂=A, G or S

X₃=N or S

X₈=M, V or AiB

X₁₁=K_{Ac}, R, K, T or A (most preferably K_{Ac}, R or K)

X₁₃=T, S or Y

X₁₄=Q or A (most preferably Q)

X₁₅=D or E

X₁₇=H or N

X₁₈=R or K

X₁₉=K_{Ac}, L, F or K (most preferably K_{Ac}, L or F)

X₂₀=Q, H or A

X₂₂=Y or F

X₂₄=K, Q or R

wherein either X₁₁ is K_{Ac} and/or X₁₉ is K_{Ac},

and wherein K_{Ac} is a lysine residue wherein the side chain ε-amino group is acylated with an acyl group selected from any one of the following:

C₁₆ or longer fatty acid,

C₁₆ or longer fatty diacid,

linker-C₁₆ or longer fatty acid, or

linker-C₁₆ or longer fatty diacid.

Preferably, X₂ is S and X₃ is N; or X₂ is G and X₃ is N; or X₂ is A and X₃ is S.

Preferably, X₁₃ is S or T, most preferably S. Preferably, X₂₄ is R or K.

In a preferred embodiment,
X₁₁ is K_{Ac}, X₁₇ is H, X₁₈ is K, X₁₉ is L and X₂₀ is Q or A; or
X₁₁ is K_{Ac}, X₁₇ is H, X₁₈ is R, X₁₉ is L and X₂₀ is Q or A; or
X₁₁ is K_{Ac}, X₁₇ is N, X₁₈ is K, X₁₉ is F and X₂₀ is H or A; or
X₁₁ is K_{Ac}, X₁₇ is N, X₁₈ is R, X₁₉ is F and X₂₀ is H or A; or
X₁₁ is R or K, X₁₇ is H, X₁₈ is K, X₁₉ is K_{Ac} and X₂₀ is Q or A; or
X₁₁ is R or K, X₁₇ is H, X₁₈ is R, X₁₉ is K_{Ac} and X₂₀ is Q or A; or
X₁₁ is R or K, X₁₇ is N, X₁₈ is K, X₁₉ is K_{Ac} and X₂₀ is H or A; or
X₁₁ is R or K, X₁₇ is N, X₁ is R, X₁₈ is K_{A}, and X₂₀ is H or A.

In a preferred embodiment, X₂ is S, X₃ is N, X₁₁ is K_{Ac}, X₁₃ is S, X₁₇ is H, X₁₈ is K or R, X₁₉ is L, X₂₀ is Q or A and X₂₂ is Y; or X₂ is S, X₃ is N, X₁₁ is R or K, X₁₃ is S, X₁₇ is H, X₁₈ is K or R, X₁₉ is K_{Ac}, X₂₀ is Q or A and X₂₂ is Y. In a preferred embodiment, X₂ is A, X₃ is S, X₁₁ is K_{Ac}, X₁₃ is S, X₁₇ is H, X₁₈ is K or R, X₁₉ is L, X₂₀ is Q or A and X₂₂ is F; or X₂ is A, X₃ is S, X₁₁ is R or K, X₁₃ is S, X₁₇ is H, X₁₈ is K or R, X₁₉ is K_{Ac}, X₂₀ is Q or A and X₂₂ is F. In a preferred embodiment, X₂ is G, X₃ is N, X₁₁ is K_{Ac}, X₁₃ is T, X₁₇ is N, X₁₈ is K or R, X₁₉ is F, X₂₀ is H or A and X₂₂ is F; or X₂ is G, X₃ is N, X₁₁ is R or K, X₁₃ is T, X₁₇ is N, X₁₈ is K or R, X₁₉ is K_{Ac}, X₂₀ is H or A and X₂₂ is F.

In another preferred aspect, the invention relates to a calcitonin mimetic, wherein the calcitonin mimetic is a 33mer peptide in accordance with formula (III):

CSNLSTCX₆LGX₇LSQDLHRX₈QTYPKX₁TX₅VGANAP (III)

or wherein the calcitonin mimetic is a 35mer peptide in accordance with formula (IV):

CSNLSTCX₆LGX₇LSQDLHRX₈QTYPKX₁X₂X₃TX₅VGANAP (IV)

or wherein the calcitonin mimetic is a 36mer peptide in accordance with formula (V):

CSNLSTCX₆LGX₇LSQDLHRX₈QTYPKX₁X₂X₃X₄TX₅VGANAP (V)

or wherein the calcitonin mimetic is a 37mer peptide in accordance with formula (VI):

CSNLSTCX₆LGK_{Ac}LZX₁X₂X₃X₄TX₅VGANAP (VI)

wherein each of X₁ to X₄ is any amino acid, with the proviso that at least one of X₁ to X₄ is a basic amino acid residue, and/or at least two of X₁ to X₄ are independently a polar amino acid residue or a basic amino acid residue, and/or at least one of X₁ to X₄ is a Gly residue, and wherein none of X₁ to X₄ is an acidic residue;

wherein X₅ is D or N;

wherein X₆ is AiB or M;

wherein either X₇ is K_{Ac} and X₈ is L, or X₇ is R or K and X₈ is K_{Ac},

wherein Z is selected from SQDLHRLSNNFGA, SQDLHRLQTYGAI or ANFLVHSSNNFGA; and

wherein K_{Ac} is a lysine residue wherein the side chain ε-amino group is acylated with an acyl group selected from any one of the following:

C₁₆ or longer fatty acid,

C₁₆ or longer fatty diacid,

linker-C₁₆ or longer fatty acid, or

linker-C₁₆ or longer fatty diacid.

Preferably, at least one of X₁ or X₄ of formulae (Ill)-(VI) is a basic amino acid residue. Preferably still, at least one of X₁ or X₄ is a basic amino acid residue, and at least one more of X₁ to X₄ is independently a polar amino acid residue or a basic amino acid residue, and none of X₁ to X₄ is an acidic residue. Preferably still, at least three of X₁ to X₄ are independently a polar amino acid residue or a basic amino acid residue, and none of X₁ to X₄ is an acidic residue. More preferably, all of X₁ to X₄ are independently a polar amino acid residue or a basic amino acid residue, and none of X₁ to X₄ is an acidic residue. Most preferably, all of X₁ to X₄ are independently a polar amino acid residue or a basic amino acid residue, at least three of X₁ to X₄ are basic amino acid residues, and none of X₁ to X₄ is an acidic residue.

The basic amino acid residues may be any natural or unnatural amino acid residues with basic side chains, and may be selected from, but are not limited to, Arg, His or Lys. The polar amino acid residues may be any natural or unnatural amino acid residues with polar uncharged side chains, and may be selected from, but are not limited to, Ser, Thr, Asn, Gln or Cys. As used herein, the term “acidic residue” refers to any natural or unnatural amino acid residue that has an acidic side chain, such as, for example, Glu or Asp.

In a preferred embodiment, X₁ is selected from Asn, Phe, Val, Gly, lie, Leu, Lys, His or Arg;

X₂ is selected from Ala, Asn, His, Leu, Ser, Thr, Gly or Lys;

X₃ is selected from Ala, Phe, lie, Ser, Pro, Thr, Gly or Lys; and/or

X₄ is selected from Ile, Leu, Gly, His, Arg, Asn, Ser, Lys, Thr or Gln;

with proviso that at least one of X₁ or X₄ is a basic amino acid residue, and/or at least two of X₁ to X₄ are independently a polar amino acid residue and/or a basic amino acid residue, and/or at least one of X₁ to X₄ is a Gly residue.

In a preferred embodiment, X₁ is selected from Asn, Gly, lie, His or Arg;

X₂ is selected from Asn, Leu, Thr, Gly or Lys;

X₃ is selected from Phe, Pro, lie, Ser, Thr, Gly or Lys; and/or

X₄ is selected from Gly, His, Asn, Ser, Lys, Thr or Gln;

with proviso that at least one of X₁ or X₄ is a basic amino acid residue, and/or at least two of X₁ to X₄ are independently a polar amino acid residue and/or a basic amino acid residue, and/or at least one of X₁ to X₄ is a Gly residue.

Peptides of the invention in accordance with formulae (Ill)-(V), supra, may comprise one or more of the following conservative substitutions:
Asp residue at position 15 of the peptide is substituted with Glu;
Arg residue at position 18 of the peptide is substituted with Lys; and/or
Lys residue at position 24 of the peptide is substituted with Arg.

Peptides of the invention in accordance with formulae (VI), supra, wherein the Z component of the peptide of formula (VI) is SQDLHRLSNNFGA or SQDLHRLQTYGAI, may comprise one or more of the following conservative substitutions:
Asp residue at position 15 of the peptide is substituted with Glu; and/or
Arg residue at position 18 of the peptide is substituted with Lys.

In all aspects of the invention, the linker preferably comprises a glutamic acid residue and/or an oligoethyleneglycol (OEG) amino acid linker comprising one OEG amino acid or two or more OEG amino acids linked together, wherein said OEG amino acid is:

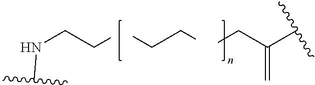

and wherein n is from 1 to 10, preferably 1 to 5, preferably 1 to 3, preferably 1 or 2, and most preferably 1.

The OEG amino acid linker may preferably comprise one OEG amino acid or two to six OEG amino acids linked together. More preferably, the OEG amino acid linker comprises one OEG amino acid, or two to three OEG amino acids linked together. Most preferably, the OEG amino acid linker comprises two OEG amino acids linked together. The OEG amino acid linker may further comprise one or more glutamic acid residues linked to the amino terminus or to the carboxyl terminus of the OEG amino acid linker. Preferably, the OEG amino acid linker is selected from any one of the following:

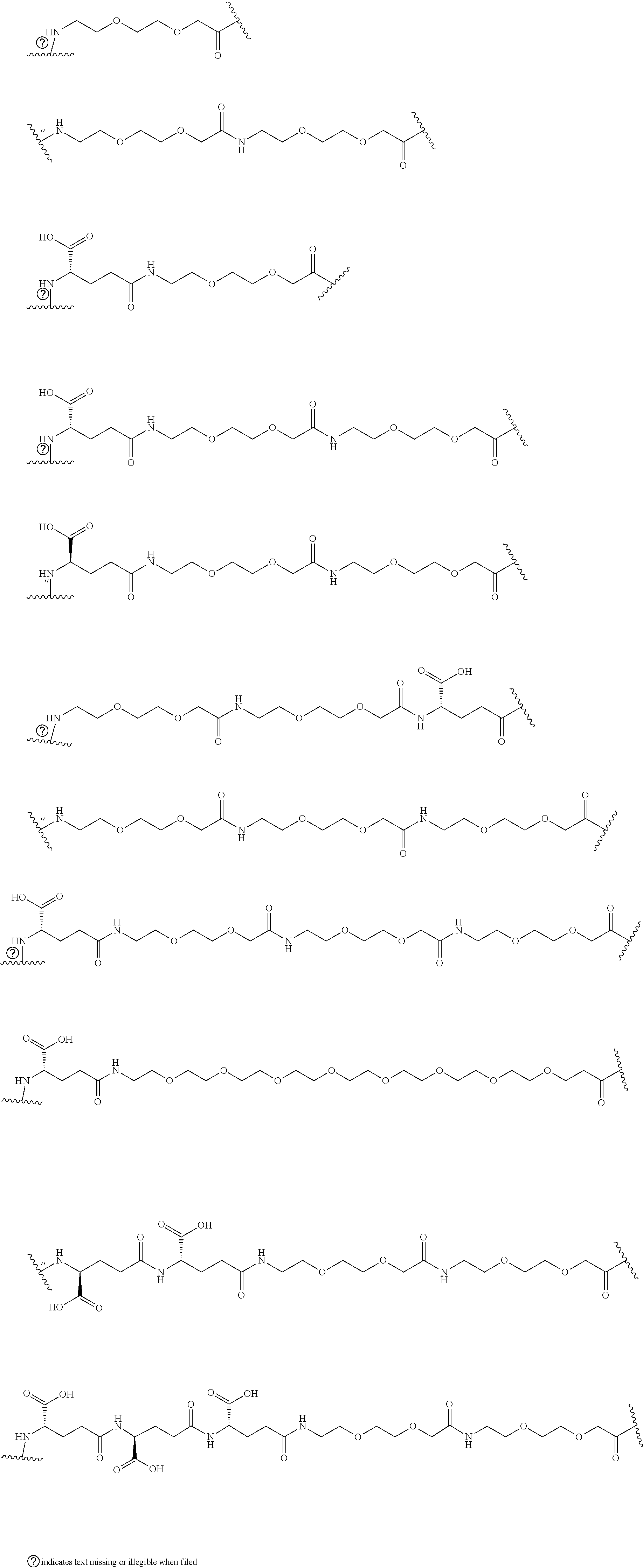

Preferably, the OEG amino acid linker is:

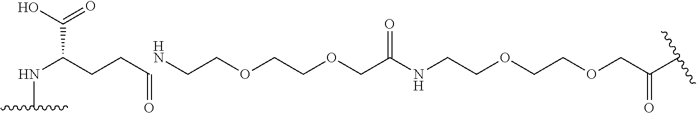

In a preferred embodiment, the acyl group is selected from C₁₈ or longer fatty acid, C₁₈ or longer fatty diacid, linker-C₁₈ or longer fatty acid, or linker-C₁₈ or longer fatty diacid. Preferably, the acyl group is selected from any one of the following: C₁₃ to C₃₀ fatty acid, preferably C₁₃ to C₂₂ fatty acid, C₁₈ to C₃₀ fatty diacid, preferably C₁₈ to C₂₂ fatty diacid, linker-C₁ to C₃₀ fatty acid, preferably linker-C₁₈ to C₂₂ fatty acid, or linker-C₁₈ to C₃₀ fatty diacid, preferably linker-C₁₈ to C₂₂ fatty acid.

Preferably, the C₁₈ fatty diacid is octadecanedioic acid (CAS No. 871-70-5).

In a preferred embodiment, K_{Ac} is acylated with a linker-fatty diacid, wherein the fatty diacid is a C₁₈ to C₂₂ fatty diacid and the linker is

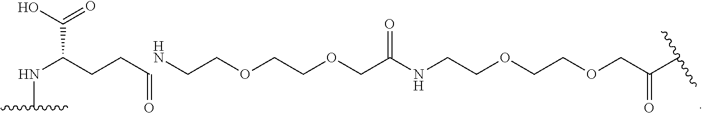

Preferably, the C₁₈ fatty diacid is octadecanedioic acid.

Preferably, the calcitonin mimetic of the invention is selected from any one of the following:

CSNLSTCMLGK_{Ac}LSQDLHRLQTYPKTDVGANAP
CSNLSTCMLGK_{Ac}LSQELHRLQTYPKTDVGANAP
CSNLSTCVLGK_{Ac}LSQELHKLQTYPRTDVGANAP
CASLSTCVLGK_{Ac}LSQDLHKLQTFPKTDVGANAP
CGNLSTCMLGK_{Ac}LSQDLNKFHTFPQTDVGANAP
CSNLSTC(AiB)LGK_{Ac}LSQDLHRLQTYPKTDVGANAP
CGNLSTC(AiB)LGK_{Ac}LTQDLNKFHTFPKTDVGANAP
CSNLSTC(AiB)LGK_{Ac}LANFLVHSSNNFGAILPKTDVGANAP
CSNLSTCMLGK_{Ac}LSQDLHRLQTYPKHTDVGANAP
CSNLSTCMLGK_{Ac}LSQDLHRLQTYPKHSSTDVGANAP
CSNLSTCMLGK_{Ac}LSQDLHRLQTYPKHSSNTDVGANAP
CSNLSTCMLGK_{Ac}LSQDLHRLSNNFGAILSSTNVGANAP
CSNLSTCMLGK_{Ac}LSQDLHRLQTYGAILSPKTDVGANAP
CSNLSTCMLGK_{Ac}LANFLVHSSNNFGAILPKTDVGANAP
CSNLSTCMLGK_{Ac}LSQDLHRLQTYPKILSSTDVGANAP
CSNLSTCMLGK_{Ac}LSQDLHRLQTYPKGLITTDVGANAP
CSNLSTCMLGK_{Ac}LSQDLHRLQTYPKNNFGTDVGANAP
CSNLSTCMLGK_{Ac}LSQDLHRLQTYPKRTTQTDVGANAP
CSNLSTCMLGK_{Ac}LSQDLHRLQTYPKHTTNTDVGANAP
CSNLSTCMLGK_{Ac}LSQDLHRLQTYPKHGGQTDVGANAP
CSNLSTCMLGK_{Ac}LSQDLHRLQTYPKHKKNTDVGANAP
CSNLSTCMLGK_{Ac}LSQDLHRLQTYPKHKKHTDVGANAP
CSNLSTC(AiB)LGRLSQDLHRK_{Ac}QTYPKTDVGANAP
CSNLSTCMLGRLSQELHRK_{Ac}QTYPKTDVGANAP

wherein K_{Ac} is as defined supra. The amino acid residue in the 8 position of the above peptides is, where not already the case, optionally substituted with AiB.

Preferably, the calcitonin mimetic of the invention is selected from any one of the following:

AcCSNLSTCMLGK_{Ac}LSQDLHRLQTYPKTDVGANAP-NH₂
AcCSNLSTC(AiB)LGK_{Ac}LSQDLHRLQTYPKTDVGANAP-NH₂
AcCGNLSTC(AiB)LGK_{Ac}LTQDLNKFHTFPKTDVGANAP-NH₂
AcCSNLSTCVLGK_{Ac}LSQELHKLQTYPRTDVGANAP-NH₂
AcCSNLSTCMLGK_{Ac}LSQELHRLQTYPKTDVGANAP-NH₂
AcCASLSTCVLGK_{Ac}LSQDLHKLQTFPKTDVGANAP-NH₂
AcCGNLSTCMLGK_{Ac}LSQDLNKFHTFPQTDVGANAP-NH₂
AcCSNLSTCMLGK_{Ac}LSQDLHRLQTYPKHTDVGANAP-NH₂
AcCSNLSTCMLGK_{Ac}LSQDLHRLQTYPKHSSTDVGANAP-NH₂
AcCSNLSTCMLGK_{Ac}LSQDLHRLQTYPKHSSNTDVGANAP-NH₂
AcCSNLSTCMLGK_{Ac}LSQDLHRLSNNFGAILSSTNVGANAP-NH₂
AcCSNLSTCMLGK_{Ac}LSQDLHRLQTYGAILSPKTDVGANAP-NH₂
AcCSNLSTCMLGK_{Ac}LANFLVHSSNNFGAILPKTDVGANAP-NH₂
AcCSNLSTCMLGK_{Ac}LSQDLHRLQTYPKILSSTDVGANAP-NH₂
AcCSNLSTCMLGK_{Ac}LSQDLHRLQTYPKGLITTDVGANAP-NH₂
AcCSNLSTCMLGK_{Ac}LSQDLHRLQTYPKNNFGTDVGANAP-NH₂
AcCSNLSTCMLGK_{Ac}LSQDLHRLQTYPKRTTQTDVGANAP-NH₂
AcCSNLSTCMLGK_{Ac}LSQDLHRLQTYPKHTTNTDVGANAP-NH₂
AcCSNLSTCMLGK_{Ac}LSQDLHRLQTYPKHGGQTDVGANAP-NH₂
AcCSNLSTCMLGK_{Ac}LSQDLHRLQTYPKHKKNTDVGANAP-NH₂
AcCSNLSTCMLGK_{Ac}LSQDLHRLQTYPKHKKHTDVGANAP-NH₂
AcCSNLSTC(AiB)LGK_{Ac}LANFLVHSSNNFGAILPKTDVGANAP-NH₂
AcCSNLSTC(AiB)LGRLSQDLHRK_{Ac}QTYPKTDVGANAP-NH₂
AcCSNLSTCMLGRLSQELHRK_{Ac}QTYPKTDVGANAP-NH₂

wherein K_{Ac} is acylated with a linker-fatty diacid, and wherein the fatty diacid is a C₁₈ to C₂₂ fatty diacid and the linker is

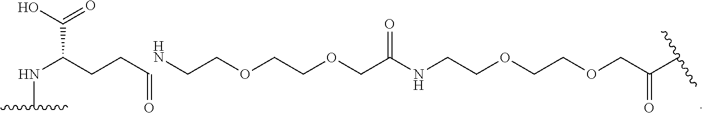

Preferably, the C₁₈ fatty diacid is octadecanedioic acid. The amino acid residue in the 8 position of the above peptides is, where not already the case, optionally substituted with AiB. In the above peptides, “Ac” indicates that the N-terminus of the peptide is acetylated, and “—NH₂” indicates that the C-terminus of the peptide is amidated.

The calcitonin mimetic of the invention may be formulated for enteral administration. For example, the calcitonin mimetic may be formulated in a pharmaceutical composition for oral administration comprising coated citric acid particles, and wherein the coated citric acid particles increase the oral bioavailability of the peptide. Alternatively, or in addition to, the calcitonin mimetic may be formulated with a carrier for oral administration. An exemplary carrier may comprise 5-CNAC, SNAD, or SNAC. The calcitonin mimetic of the invention may also be formulated for parenteral administration. For example, the calcitonin mimetic may be formulated for injection.

The present invention also relates to a pharmaceutical composition comprising a calcitonin mimetic as described supra.

The present invention also relates to a calcitonin mimetic as described supra for use as a medicament. In that regard, the calcitonin mimetic may be for use in treating diabetes (Type I and/or Type II), excess bodyweight, excessive food consumption, metabolic syndrome, rheumatoid arthritis, non-alcoholic steatohepatitis (NASH), non-alcoholic fatty liver disease, alcoholic fatty liver disease, osteoporosis, or osteoarthritis, poorly regulated blood glucose levels, poorly regulated response to glucose tolerance tests, or poor regulation of food intake. The calcitonin mimetic may also be administered in conjunction with metformin or another insulin sensitizer.

The peptides of the invention may be acylated at its N-terminal or otherwise modified to reduce the positive charge of the first amino acid and independently of that may be amidated at its C-terminal.

The peptide may be formulated for administration as a pharmaceutical and may be formulated for enteral or parenteral administration. Preferred formulations are injectable, preferably for subcutaneous injection, however the peptide may be formulated with a carrier for oral administration, and optionally wherein the carrier increases the oral bioavailability of the peptide. Suitable carriers include ones that comprise 5-CNAC, SNAD, or SNAC.

Optionally, the peptide is formulated in a pharmaceutical composition for oral administration comprising coated citric acid particles, and wherein the coated citric acid particles increase the oral bioavailability of the peptide.

The invention includes a peptide of the invention for use as a medicament. The peptide may be for use in treating diabetes (Type I and/or Type II), excess bodyweight, excessive food consumption, metabolic syndrome, rheumatoid arthritis, non-alcoholic steatohepatitis (NASH), non-alcoholic fatty liver disease, alcoholic fatty liver disease, osteoporosis, or osteoarthritis, poorly regulated blood glucose levels, poorly regulated response to glucose tolerance tests, or poor regulation of food intake. In particular, the peptides may be used to lower an undesirably high fasting blood glucose level or to lower an undesirably high HbA1c or to reduce an undesirably high response to a glucose tolerance test. The peptides of the invention may also be used for producing a decrease in liver triglycerides and/or for reducing fat accumulation in the liver of a subject.

The peptides of the invention may be produced using any suitable method known in the art for generating peptides, such as synthetic (chemical) and recombinant technologies. Preferably, the peptides are produced using a synthetic method. Synthetic peptide synthesis is well known in the art, and includes (but is not limited to) solid phase peptide synthesis employing various protecting group strategies (e.g. using Fmoc, Boc, Bzl, tBu, etc.).

In some embodiments, the N-terminal side of the calcitonin mimetics discussed supra is modified to reduce the positive charge of the first amino acid. For example, an acetyl, propionyl, or succinyl group may be substituted on cysteine-1. Alternative ways of reducing positive charge include, but are not limited to, polyethylene glycol-based PEGylation, or the addition of another amino acid such as glutamic acid or aspartic acid at the N-terminus. Alternatively, other amino acids may be added to the N-terminus of peptides discussed supra including, but not limited to, lysine, glycine, formylglycine, leucine, alanine, acetyl alanine, and dialanyl. As those of skill in the art will appreciate, peptides having a plurality of cysteine residues frequently form a disulfide bridge between two such cysteine residues. All such peptides set forth herein are defined as optionally including one or more such disulphide bridges, particularly at the Cys1-Cys7 locations. Mimicking this, the cysteines at positions 1 and 7 may jointly be replaced by an α-aminosuberic acid linkage. While calcitonin mimetics of the present disclosure may exist in free acid form, it is preferred that the C-terminal amino acid be amidated. Applicants expect that such amidation may contribute to the effectiveness and/or bioavailability of the peptide. Synthetic chemical methods may be employed for amidating the C-terminal amino acid. Another technique for manufacturing amidated versions of the calcitonin mimetics of the present disclosure is to react precursors (having glycine in place of the C-terminal amino group of the desired amidated product) in the presence of peptidylglycine alpha-amidating monooxygenase in accordance with known techniques wherein the precursors are converted to amidated products in reactions described, for example, in U.S. Pat. No. 4,708,934 and EP0308067 and EP0382403.

Production of amidated products may also be accomplished using the process and amidating enzyme set forth by Consalvo, et al in U.S. Pat. No. 7,445,911; Miller et al, US2006/0292672; Ray et al, 2002, Protein Expression and Purification, 26:249-259; and Mehta, 2004, Biopharm. International, July, pp. 44-46.

The production of the preferred amidated peptides may proceed, for example, by producing glycine-extended precursor in E. coli as a soluble fusion protein with glutathione-S-transferase, or by direct expression of the precursor in accordance with the technique described in U.S. Pat. No. 6,103,495. Such a glycine extended precursor has a molecular structure that is identical to the desired amidated product except at the C-terminus (where the product terminates —X—NH₂, while the precursor terminates -X-gly, X being the C-terminal amino acid residue of the product). An alpha-amidating enzyme described in the publications above catalyzes conversion of precursors to product. That enzyme is preferably recombinantly produced, for example, in Chinese Hamster Ovary (CHO) cells), as described in the Biotechnology and Biopharm. articles cited above.

Free acid forms of peptide active agents of the present disclosure may be produced in like manner, except without including a C-terminal glycine on the “precursor”, which precursor is instead the final peptide product and does not require the amidation step.

Except where otherwise stated, the preferred dosage of the calcitonin mimetics of the present disclosure is identical for both therapeutic and prophylactic purposes. Desired dosages are discussed in more detail, infra, and differ depending on mode of administration.

Except where otherwise noted or where apparent from context, dosages herein refer to weight of active compounds (i.e. calcitonin mimetics) unaffected by or discounting pharmaceutical excipients, diluents, carriers or other ingredients, although such additional ingredients are desirably included. Any dosage form (capsule, tablet, injection or the like) commonly used in the pharmaceutical industry for delivery of peptide active agents is appropriate for use herein, and the terms “excipient”, “diluent”, or “carrier” includes such non-active ingredients as are typically included, together with active ingredients in such dosage form in the industry. A preferred oral dosage form is discussed in more detail, infra, but is not to be considered the exclusive mode of administering the active agents of the present disclosure.

The calcitonin mimetics of the present disclosure can be administered to a patient to treat a number of diseases or disorders. As used herein, the term “patient” means any organism belonging to the kingdom Animalia. In an embodiment, the term “patient” refers to vertebrates, more preferably, mammals including humans.

Accordingly, the present disclosure includes the use of the peptides in a method of treatment of type I diabetes, Type II diabetes or metabolic syndrome, obesity, or of appetite suppression, or for mitigating insulin resistance, or for reducing an undesirably high fasting serum glucose level, or for reducing an undesirably high peak serum glucose level, or for reducing an undesirably high peak serum insulin level, or for reducing an undesirably large response to a glucose tolerance test, or for treating osteoporosis, or for treating osteoarthritis, or for treating non-alcoholic steatohepatitis (NASH), or for treating alcoholic fatty liver disease, or for producing a decrease in liver triglycerides, or for reducing fat accumulation in the liver of a subject.

There are a number of art-recognized measures of normal range for body weight in view of a number of factors such as gender, age and height. A patient in need of treatment or prevention regimens set forth herein include patients whose body weight exceeds recognized norms or who, due to heredity, environmental factors or other recognized risk factor, are at higher risk than the general population of becoming overweight or obese. In accordance with the present disclosure, it is contemplated that the calcitonin mimetics may be used to treat diabetes where weight control is an aspect of the treatment.

In an embodiment, the method includes enteral administration to a patient in need thereof for treatment of a said condition of a pharmaceutically effective amount of any one of the peptides described herein.

In an embodiment, the method includes parenteral administration to a patient in need thereof for treatment of a said condition of a pharmaceutically effective amount of any one of the peptides described herein. For parenteral administration (including intraperitoneal, subcutaneous, intravenous, intradermal or intramuscular injection), solutions of a peptide of the present disclosure in either sesame or peanut oil or in aqueous propylene glycol may be employed, for example. The aqueous solutions should be suitably buffered if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art. For parenteral application, examples of suitable preparations include solutions, preferably oily or aqueous solutions as well as suspensions, emulsions, or implants, including suppositories. Peptides may be formulated in sterile form in multiple or single dose formats such as being dispersed in a fluid carrier such as sterile physiological saline or 5% saline dextrose solutions commonly used with injectables.

Said method may include a preliminary step of determining whether the patient suffers from a said condition, and/or a subsequent step of determining to what extent said treatment is effective in mitigating the condition in said patient, e.g. in each case, carrying out an oral glucose tolerance test or a resting blood sugar level.

Oral enteral formulations are for ingestion by swallowing for subsequent release in the intestine below the stomach, and hence delivery via the portal vein to the liver, as opposed to formulations to be held in the mouth to allow transfer to the bloodstream via the sublingual or buccal routes.

Suitable dosage forms for use in the present disclosure include tablets, mini-tablets, capsules, granules, pellets, powders, effervescent solids and chewable solid formulations. Such formulations may include gelatin which is preferably hydrolysed gelatin or low molecular weight gelatin. Such formulations may be obtainable by freeze drying a homogeneous aqueous solution comprising a calcitonin mimetic and hydrolysed gelatin or low molecular weight gelatin and further processing the resulting solid material into said oral pharmaceutical formulation, and wherein the gelatin may have a mean molecular weight from 1000 to 15000 Daltons. Such formulations may include a protective carrier compound such as 5-CNAC or others as disclosed herein.

Whilst oral formulations such as tablets and capsules are preferred, compositions for use in the present disclosure may take the form of syrups, elixirs or the like and suppositories or the like. Oral delivery is generally the delivery route of choice since it is convenient, relatively easy and generally painless, resulting in greater patient compliance relative to other modes of delivery. However, biological, chemical and physical barriers such as varying pH in the gastrointestinal tract, powerful digestive enzymes, and active agent impermeable gastrointestinal membranes, makes oral delivery of calcitonin like peptides to mammals problematic, e.g. the oral delivery of calcitonins, which are long-chain polypeptide hormones secreted by the parafollicular cells of the thyroid gland in mammals and by the ultimobranchial gland of birds and fish, originally proved difficult due, at least in part, to the insufficient stability of calcitonin in the gastrointestinal tract as well as the inability of calcitonin to be readily transported through the intestinal walls into the blood stream.

Suitable oral formulations are however described below.

### Treatment of Patients

In an embodiment, a calcitonin mimetic of the present disclosure is administered at adequate dosage to maintain serum levels of the mimetic in patients between 5 picograms and 1000 nanograms per milliliter, preferably between 50 picograms and 500 nanograms, e.g. between 1 and 300 nanograms per milliliter. The serum levels may be measured by any suitable techniques known in the art, such as radioimmunoassay or mass spectrometry. The attending physician may monitor patient response, and may then alter the dosage somewhat to account for individual patient metabolism and response. Near simultaneous release is best achieved by administering all components of the present disclosure as a single pill or capsule. However, the disclosure also includes, for example, dividing the required amount of the calcitonin mimetic among two or more tablets or capsules which may be administered together such that they together provide the necessary amount of all ingredients. “Pharmaceutical composition,” as used herein includes but is not limited to a complete dosage appropriate to a particular administration to a patient regardless of whether one or more tablets or capsules (or other dosage forms) are recommended at a given administration.

A calcitonin mimetic of the present disclosure may be formulated for oral administration using the methods employed in the Unigene Enteripep® products. These may include the methods as described in U.S. Pat. Nos. 5,912,014, 6,086,918, 6,673,574, 7,316,819, 8,093,207, and US Publication No. 2009/0317462. In particular, it may include the use of conjugation of the compound to a membrane translocator such as the protein transduction domain of the HIV TAT protein, co-formulation with one or more protease inhibitors, and/or a pH lowering agent which may be coated and/or an acid resistant protective vehicle and/or an absorption enhancer which may be a surfactant.

In an embodiment, a calcitonin mimetic of the present disclosure is preferably formulated for oral delivery in a manner known in U.S. Patent Publication No. 2009/0317462.

In an embodiment, a calcitonin mimetic of the present disclosure may be formulated for enteral, especially oral, administration by admixture with a suitable carrier compound. Suitable carrier compounds include those described in U.S. Pat. Nos. 5,773,647 and 5,866,536 and amongst these, 5-CNAC (N-(5-chlorosalicyloyl)-8-aminocaprylic acid, commonly as its disodium salt) is particularly effective. Other preferred carriers or delivery agents are SNAD (sodium salt of 10-(2-Hydroxybenzamido)decanoic acid) and SNAC (sodium salt of N-(8-[2-hydroxybenzoyl]amino)caprylic acid). In an embodiment, a pharmaceutical composition of the present disclosure comprises a delivery effective amount of carrier such as 5-CNAC, i.e. an amount sufficient to deliver the compound for the desired effect. Generally, the carrier such as 5-CNAC is present in an amount of 2.5% to 99.4% by weight, more preferably 25% to 50% by weight of the total composition.

In addition, WO 00/059863 discloses the disodium salts of formula I

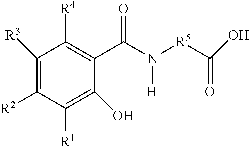

wherein
R¹, R², R³, and R⁴ are independently hydrogen, —OH, —NR⁶R⁷, halogen, C₁-C₄ alkyl, or C₁-C₄alkoxy;
R⁵ is a substituted or unsubstituted C₂-C₁₆ alkylene, substituted or unsubstituted C₂-C₁₆ alkenylene, substituted or unsubstituted C₁-C₁₂ alkyl(arylene), or substituted or unsubstituted aryl(C₁-C₁₂ alkylene); and R⁶ and R⁷ are independently hydrogen, oxygen, or C₁-C₄ alkyl; and hydrates and solvates thereof as particularly efficacious for the oral delivery of active agents, such as calcitonins, e.g. salmon calcitonin, and these may be used in the present disclosure.

Preferred enteric formulations using optionally micronised 5-CNAC may be generally as described in WO2005/014031.

The compound may be formulated for oral administration using the methods employed in the Capsitonin product of Bone Medical Limited. These may include the methods incorporated in Axcess formulations. More particularly, the active ingredient may be encapsulated in an enteric capsule capable of withstanding transit through the stomach. This may contain the active compound together with a hydrophilic aromatic alcohol absorption enhancer, for instance as described in WO02/028436. In a known manner the enteric coating may become permeable in a pH sensitive manner, e.g. at a pH of from 3 to 7. WO2004/091584 also describes suitable formulation methods using aromatic alcohol absorption enhancers.

The compound may be formulated using the methods seen in the Oramed products, which may include formulation with omega-3 fatty acid as seen in WO2007/029238 or as described in U.S. Pat. No. 5,102,666.

Generally, the pharmaceutically acceptable salts (especially mono or di sodium salts), solvates (e.g. alcohol solvates) and hydrates of these carriers or delivery agents may be used.

Oral administration of the pharmaceutical compositions according to the disclosure can be accomplished regularly, e.g. once or more on a daily or weekly basis; intermittently, e.g. irregularly during a day or week; or cyclically, e.g. regularly for a period of days or weeks followed by a period without administration. The dosage form of the pharmaceutical compositions of the presently disclosed embodiments can be any known form, e.g. liquid or solid dosage forms. The liquid dosage forms include solution emulsions, suspensions, syrups and elixirs. In addition to the active compound and carrier such as 5-CNAC, the liquid formulations may also include inert excipients commonly used in the art such as, solubilizing agents e.g. ethanol; oils such as cottonseed, castor and sesame oils; wetting agents; emulsifying agents; suspending agents; sweeteners; flavourings; and solvents such as water. The solid dosage forms include capsules, soft-gel capsules, tablets, caplets, powders, granules or other solid oral dosage forms, all of which can be prepared by methods well known in the art. The pharmaceutical compositions may additionally comprise additives in amounts customarily employed including, but not limited to, a pH adjuster, a preservative, a flavorant, a taste-masking agent, a fragrance, a humectant, a tonicifier, a colorant, a surfactant, a plasticizer, a lubricant such as magnesium stearate, a flow aid, a compression aid, a solubilizer, an excipient, a diluent such as microcrystalline cellulose, e.g. Avicel PH 102 supplied by FMC corporation, or any combination thereof. Other additives may include phosphate buffer salts, citric acid, glycols, and other dispersing agents. The composition may also include one or more enzyme inhibitors, such as actinonin or epiactinonin and derivatives thereof; aprotinin, Trasylol and Bowman-Birk inhibitor. Further, a transport inhibitor, i.e. a [rho]-glycoprotein such as Ketoprofin, may be present in the compositions of the present disclosure. The solid pharmaceutical compositions of the instant disclosure can be prepared by conventional methods e.g. by blending a mixture of the active compound, the carrier such as 5-CNAC, and any other ingredients, kneading, and filling into capsules or, instead of filling into capsules, molding followed by further tableting or compression-molding to give tablets. In addition, a solid dispersion may be formed by known methods followed by further processing to form a tablet or capsule. Preferably, the ingredients in the pharmaceutical compositions of the instant disclosure are homogeneously or uniformly mixed throughout the solid dosage form.

Alternatively, the active compound may be formulated as a conjugate with said carrier, which may be an oligomer as described in US2003/0069170, e.g.

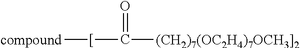

Such conjugates may be administered in combination with a fatty acid and a bile salt as described there.

Conujugates with polyethylene glycol (PEG) may be used, as described for instance in Mansoor et al.

Alternatively, active compounds may be admixed with nitroso-N-acetyl-D,L-penicillamine (SNAP) and Carbopol solution or with taurocholate and Carbapol solution to form a mucoadhesive emulsion.

The active compound may be formulated by loading into chitosan nanocapsules as disclosed in Prego et al (optionally PEG modified as in Prego Prego C, Torres D, Fernandez-Megia E, Novoa-Carballal R, Quino& E, Alonso M J.) or chitosan or PEG coated lipid nanoparticles as disclosed in Garcia-Fuentes et al. Chitosan nanoparticles for this purpose may be iminothiolane modified as described in Guggi et al. They may be formulated in water/oil/water emulsions as described in Dogru et al. The bioavailability of active compounds may be increased by the use of taurodeoxycholate or lauroyl carnitine as described in Sinko et al or in Song et al. Generally, suitable nanoparticles as carriers are discussed in de la Fuente et al and may be used in the present disclosure.

Other suitable strategies for oral formulation include the use of a transient permeability enhancer (TPE) system as described in WO2005/094785 of Chiasma Ltd. TPE makes use of an oily suspension of solid hydrophilic particles in a hydrophobic medium to protect the drug molecule from inactivation by the hostile gastrointestinal (GI) environment and at the same time acts on the GI wall to induce permeation of its cargo drug molecules.

Further included is the use of glutathione or compounds containing numerous thiol groups as described in US2008/0200563 to inhibit the action of efflux pumps on the mucous membrane. Practical examples of such techniques are described also in Caliceti, P. Salmaso, S., Walker, G. and Bernkop-Schnurch, A. (2004) ‘Development and in vivo evaluation of an oral insulin-PEG delivery system.’ Eur. J. Pharm. Sci., 22, 315-323, in Guggi, D., Krauland, A. H., and Bernkop-Schnurch, A. (2003) ‘Systemic peptide delivery via the stomach: in vivo evaluation of an oral dosage form for salmon calcitonin’. J. Control. Rel. 92, 125-135, and in Bernkop-Schnurch, A., Pinter, Y., Guggi, D., Kahlbacher, H., Schöffmann, G., Schuh, M., Schmerold, I., Del Curto, M. D., D'Antonio, M., Esposito, P. and Huck, Ch. (2005) ‘The use of thiolated polymers as carrier matrix in oral peptide delivery’—Proof of concept. J. Control. Release, 106, 26-33.

The active compound may be formulated in seamless micro-spheres as described in WO2004/084870 where the active pharmaceutical ingredient is solubilised as an emulsion, microemulsion or suspension formulated into mini-spheres; and variably coated either by conventional or novel coating technologies. The result is an encapsulated drug in “pre-solubilised” form which when administered orally provides for predetermined instant or sustained release of the active drug to specific locations and at specific rates along the gastrointestinal tract. In essence, pre-solubilization of the drug enhances the predictability of its kinetic profile while simultaneously enhancing permeability and drug stability.

One may employ chitosan coated nanocapsules as described in US2009/0074824. The active molecule administered with this technology is protected inside the nanocapsules since they are stable against the action of the gastric fluid. In addition, the mucoadhesive properties of the system enhances the time of adhesion to the intestine walls (it has been verified that there is a delay in the gastrointestinal transit of these systems) facilitating a more effective absorption of the active molecule.

Methods developed by TSRI Inc. may be used. These include Hydrophilic Solubilization Technology (HST) in which gelatin, a naturally derived collagen extract carrying both positive and negative charges, coats the particles of the active ingredient contained in lecithin micelles and prevents their aggregation or clumping. This results in an improved wettability of hydrophobic drug particles through polar interactions. In addition, the amphiphilic lecithin reduces surface tension between the dissolution fluid and the particle surface.

The active ingredient may be formulated with cucurbiturils as excipients.

Alternatively, one may employ the GIPET technology of Merrion Pharmaceuticals to produce enteric coated tablets containing the active ingredient with an absorption enhancer which may be a medium chain fatty acid or a medium chain fatty acid derivative as described in US2007/0238707 or a membrane translocating peptide as described in U.S. Pat. No. 7,268,214.

One may employ GIRES™ technology which consists of a controlled-release dosage form inside an inflatable pouch, which is placed in a drug capsule for oral administration. Upon dissolution of the capsule, a gas-generating system inflates the pouch in the stomach. In clinical trials the pouch has been shown to be retained in the stomach for 16-24 hours.

Alternatively, the active may be conjugated to a protective modifier that allows it to withstand enzymatic degradation in the stomach and facilitate its absorption. The active may be conjugated covalently with a monodisperse, short-chain methoxy polyethylene glycol glycolipids derivative that is crystallized and lyophilized into the dry active pharmaceutical ingredient after purification. Such methods are described in U.S. Pat. No. 5,438,040 and at www.biocon.com.

One may also employ a hepatic-directed vesicle (HDV) for active delivery. An HDV may consist of liposomes (5150 nm diameter) encapsulating the active, which also contain a hepatocyte-targeting molecule in their lipid bilayer. The targeting molecule directs the delivery of the encapsulated active to the liver cells and therefore relatively minute amounts of active are required for effect. Such technology is described in US2009/0087479 and further at www.diasome.com.

The active may be incorporated into a composition containing additionally a substantially non-aqueous hydrophilic medium comprising an alcohol and a cosolvent, in association with a medium chain partial glyceride, optionally in admixture with a long-chain PEG species as described in US2002/0115592 in relation to insulin.

Alternatively, use may be made of intestinal patches as described in Shen Z, Mitragotri S, Pharm Res. 2002 April; 19(4):391-5 ‘Intestinal patches for oral drug delivery’.

The active may be incorporated into an erodible matrix formed from a hydrogel blended with a hydrophobic polymer as described in U.S. Pat. No. 7,189,414.

Suitable oral dosage levels for adult humans to be treated may be in the range of 0.05 to 5 mg, preferably about 0.1 to 2.5 mg.

The frequency of dosage treatment of patients may be from one to four times weekly, preferably one to two times weekly, and most preferably once weekly. Treatment will desirably be maintained over a prolonged period of at least 6 weeks, preferably at least 6 months, preferably at least a year, and optionally for life.

Combination treatments for relevant conditions may be carried out using a composition according to the present disclosure and separate administration of one or more other therapeutics. Alternatively, the composition according to the present disclosure may incorporate one or more other therapeutics for combined administration.

Combination therapies according to the present disclosure include combinations of an active compound as described with insulin, GLP-2, GLP-1, GIP, or amylin, or generally with other anti-diabetics. Thus combination therapies including co-formulations may be made with insulin sensitizers including biguanides such as Metformin, Buformin and Phenformin, TZD's (PPAR) such as Balaglitazone, Pioglitazone, Rivoglitazone, Rosiglitazone and Troglitazone, dual PPAR agonists such as Aleglitazar, Muraglitazar and Tesaglitazar, or secretagogues including sulphonylureas such as Carbutamide, Chloropropamide, Gliclazide, Tolbutamide, Tolazamide, Glipizide, Glibenclamide, Glyburide, Gliquidone, Glyclopyramide and Glimepriride, Meglitinides/glinides (K+) such as Nateglinide, Repaglinide and Mitiglinide, GLP-1 analogs such as Exenatide, Lixisenatide, Liraglutide, Semaglutide, dulaglutide and Albiglutide, DPP-4 inhibitors such as Alogliptin, Linagliptin, Saxagliptin, Sitagliptin and Vildagliptin, insulin analogs or special formulations such as (fast acting) Insulin lispro, Insulin aspart, Insulin glulisine, (long acting) Insulin glargine, Insulin detemir), inhalable insulin—Exubra and NPH insulin, and others including alpha-glucosidase inhibitors such as Acarbose, Miglitol and Voglibose, amylin analogues such as Pramlintide, SGLT2 inhibitors such as Dapagliflozin, Empagliflozin, Remogliflozin and Sergliflozin as well as miscellaneous ones including Benfluorex and Tolrestat.

Further combinations include co-administration or co-formulation with leptins. Leptin resistance is a well-established component of type 2 diabetes; however, injections of leptin have so far failed to improve upon this condition. In contrast, there is evidence supporting that amylin, and thereby molecules with amylin-like abilities, as the salmon calcitonin mimetics, are able to improve leptin sensitivity. Amylin/leptin combination has shown a synergistic effect on body weight and food intake, and also insulin resistance [Kusakabe T et al].

A further preferred combination therapy includes co-formulation or co-administration of the peptides of the invention with one or more weight loss drugs. Such weight loss drugs include, but are not limited to, lipase inhibitors (e.g. pancreatic lipase inhibitors, such as Orlistat), appetite suppressing amphetamine derivatives (e.g. Phentermine), Topiramate, Qysmia® (Phentermine/Topiramate combination), 5-HT₂c receptor agonists (e.g. Locaserin), Contrave® (naltrexone/bupropion combination), glucagon-like peptide-1 [GLP-1] analogues and derivatives (e.g. Liraglutide, semaglutide), sarco/endoplasmic reticulum (SR) Ca²⁺ ATPase (SERCA) inhibitors (e.g. sarcolipin), Fibroblast growth factor 21 [FGF-21] receptor agonists (e.g. analogs of FGF-21), and R₃ adreno receptor agonists (e.g. Mirabegron). Such combinations may be used to treat an overweight condition, such as obesity.

### DESCRIPTION OF THE FIGURES

FIG. 1: Comparison of KBP346, KBP347, KBP349, KBP351, KBP352, KBP353 and KBP089 on food intake and body weight. A) Food intake, 0-4 hours. B) Body weight change, 4 hours. C) Food intake, 4-24 hours. D) Body weight change, 24 hours. E) Food intake, 24-49 hours. F) Body weight change, 48 hours.

FIG. 2: Single dose test of KBP375, KBP376 and KBP377. Single dose was given at t=0 and the effect on food intake and body weight of a single dose 36 nmol/kg of each molecule were monitored for 168 hours and compared head-to-head with a non-acylated benchmark. A) Food intake. B) Body weight change.

FIG. 3: Dose response test of KBP356, KB358, KBP362, KBP364, KB368 and KBP370. Single dose was given at t=0 and the effect on food intake and body weight of a single dose 36 nmol/kg of each molecule were monitored for 168 hours. A-B) Food intake and Body weight of acylated KBP-066 variants. C-D) Food intake and Body weight of acylated KBP-062 variants. E-F) Food intake and Body weight of acylated KBP-110 variants.

FIG. 4: Effect of a single high dose KBP372 and KBP356 on food intake and body weight. A) KBP-042A11.03 (KBP372) effect on food intake. B) KBP-042A11.03 (KBP372) effect on body weight. C) KBP-066A11.03 (KBP356) effect on food intake. D) KBP-066A11.03 (KBP356) effect on body weight.

FIG. 5: 4 hour food intake study for KBP350.

FIG. 6: Accumulated food intake. A) Accumulated food intake over time. Food intake is monitored once daily for the initial 21 days of the study. n=3-4 cages.+/−SEM. B) Total area under the curve of the data presented in FIG. 9A. n=9-10.+/−SEM.

FIG. 7: ZDF Body weight during study. A) Body weight of individual rats in grams B) Body weight normalised to vehicle in percent. Body weight is recorded daily throughout the first 21 days, then twice weekly until one week prior to study end (day 62). The body weight of the KBP-066A11.03 group was monitored daily until one week prior to study end (day 62). n=9-10 rats.+/−SEM.

FIG. 8: ZDF Fasting blood glucose. Fasting blood glucose is measured after 6 h of fasting on day 0, 14, 28, 42 and 62 after study start. n=9-10.+/−SEM.

FIG. 9: ZDF HbA1c values. A) HbA1c at baseline. B) HbA1c at study end. HbA1c is measured on day −3 from study start. HbA1c is measured at study end, day 62. n=9-10. +/−SEM.

FIG. 10: Oral glucose tolerance test (OGTT). A) an OGTT over 180 min in male ZDF rats. B) Total area under the curve during the OGTT shown in A). The OGTT is performed after 8 weeks of treatment. The rats were fasted for 11 h prior to time point −30 minutes. Blood glucose levels are measured at time point −30, 0, 15, 30, 60, 120 and 180 minutes. Glucose is administered orally at time point 0 minutes. Blood glucose values above 33.3 mmol*L⁻¹ were assigned with the upper limit of detection; 33.3 mmol*L⁻¹. The rats had not been pre-dosed with saline or KBP-066 og KBP-066A on that same day. n=9-10. +/−SEM.

FIG. 11: Single dose test of KBP-305, KBP-306, KBP-307, KBP-356, KBP-381, KBP-382 and KBP-383. Single dose was given at t=0 and the effect on food intake and body weight of a single dose 3 nmol/kg of each molecule were monitored for 96 hours and compared head-to-head with one another and vehicle to determine the optimal acylation length. A) Acute food intake in grams(g). B) Body weight change in grams(g). n=4 rats per group. Data as +/−SEM.

FIG. 12: Six-week body weight loss study in HFD SD rats using KBP-066A11 compounds with different acylation length, 0.03, 0.04, and 0.05 acylations. Rats were treated with treated with KBP-066A11.03, KBP-066A11.04, KBP-066A11.05 or vehicle and dosed every 3^{rd} day with a single s.c. injection of 4 nmol compound/kg. Body weight is recorded daily throughout the study. A) Daily food intake in grams(g) B) Body weight loss of individual rats in grams(g). n=6 rats per group. Data as +/−SEM.

FIG. 13: Additional parameters from the body weight loss study in HFD SD rats using KBP-066A11 compounds with different acylation length, 0.03, 0.04, or 0.05 acylation. Rats were treated with KBP-066A11.03, KBP-066A11.04, KBP-066A11.05 or vehicle. Rats were dosed every 3^{rd} day with a single s.c. injection of 4 nmol compound/kg. A) Oral glucose tolerance test. B) Incremental area under the curve of the OGTT. C) Weight of the epididymal WAT at study end in grams(g). D) Weight of the inguinal WAT at study end in grams(g). E) Weight of the perirenal WAT at study end in grams(g). F) Change in body weight at study end from baseline in grams(g). Body weight was recorded daily throughout the study. n=6 rats per group. Data as +/−SEM.

FIG. 14: Competitive ligand binding assay using radio labelled salmon calcitonin (¹²⁵I-sCT) as tracer and conducted 2% serum albumin from two different species, rat (Rattus norvegicus) and man (Homo sapiens). As a tracer 0.25 nM ¹²⁵I-sCT was used. A) Competitive binding assay conducted in 2% RSA. B) Competitive binding assay conducted in 2% HSA. Data as +/−SEM.

FIG. 15: Single dose test of KBP-356, KBP-386, KBP-387, KBP-388, KBP-389, and KBP-390 for investigating the acylation position of the KBP-066 backbone. Single dose was given at t=0 and the effect on food intake and body weight of a single dose 3 nmol/kg of each molecule were monitored for 96 hours and compared head-to-head with one another and vehicle to determine the optimal acylation position. A) Acute food intake in grams(g). B) Body weight change in grams(g). n=4 rats per group. Data as +/−SEM.

FIG. 16: Single dose test of KBP-391, KBP-312, KBP-313, KBP-314, KBP-315, KBP-316, KBP-317, and KBP-318 for investigating acylation position of the KBP-021 backbone. Single dose was given at t=0 and the effect on food intake and body weight of a single dose 3 nmol/kg of each molecule were monitored for 96 hours and compared head-to-head with one another or vehicle to determine the optimal acylation position. A) Acute food intake in grams(g). B) Body weight change in grams(g). n=4 rats per group. Data as +/−SEM.

FIG. 17: Six-week body weight loss study in HFD SD rats using KBP-066 compounds with same acylation length, 0.03, but different position, A11 and A19. Rats were dosed every 3^{rd} day with a single s.c. injection of 4 nmol compound/kg KBP-066A11.03 (KBP-356), KBP-066A19.03 (KBP-389) or vehicle. Body weight and food intake was recorded daily throughout the study. A) Daily food intake during the study in grams(g). B) Body weight loss of individual rats in grams(g). n=6 rats per group. Data as +/−SEM.

FIG. 18: Additional parameters from the body weight loss study in HFD SD rats using KBP-066A11 compounds with different acylation length, 0.03, 0.04, and 0.05 acylations. Rats were treated with KBP-066A11.03, or KBP-066A19.03 or vehicle. Rats were dosed every 3^{rd} day with a single s.c. injection of 4 nmol compound/kg. A) Oral glucose tolerance test. B) Incremental area under the curve of the OGTT. C) Weight of the epididymal WAT at study end in grams(g). D) Weight of the inguinal WAT at study end in grams(g). E) Weight of the perirenal WAT at study end in grams(g). F) Change in body weight at study end from baseline in grams(g). Body weight is recorded daily throughout the study. n=6 rats per group. Data as +/−SEM.

FIG. 19: Investigating acylation linker of the KBP-066 backbone using single dose test of KBP-356, KBP-384, and KBP-385. Single dose was given at t=0 and the effect on body weight of a single dose of 4 nmol/kg of each molecule were monitored for 96 hours and compared head-to-head with one another to determine the optimal acylation linker A) Acute food intake in grams(g). B) Body weight change in grams(g). n=4 rats per group. Data as +/−SEM.

### EXAMPLES

The presently disclosed embodiments described in the following Examples, which are set forth to aid in the understanding of the disclosure, should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. In the following examples, the following materials and methods were employed.

Cells and Cell Lines

The following cell lines expressing the calcitonin, amylin and CGRP receptors were purchased and cultured according to the manufacturer's instructions.
1. Calcitonin Receptor (CTR): U2OS-CALCR from DiscoveRx (Cat. No.: 93-0566C3).
2. Amylin Receptor (AMY-R): CHO-K1 CALCR+RAMP3 from DiscoveRx (Cat. No.: 93-0268C2).

Chemicals

Thioflavin T (T3516, Sigma). Assay stock ThT is prepared as a 10 mM solution in 5 mM sodium phosphate pH 7.2. Aliquots are stored, protected from light, at −20° C. Stock ThT is thawed and diluted just prior to use.

For the tested calcitonin mimetics (hereinafter referred to as “acylated KBPs” or simply “KBPs”), final buffer conditions are 10 mM Tris-HCl pH 7.5.

The final peptide concentration in the wells should be 100-200 μM, and the final ThT concentration should be 4 μM. ThT is added last (10 μL).

Animal Models

In the animal model studies, 12 week healthy Sprague Dawley (SD) rats were used to assess the potency of the acylated KBPs. In some examples they were fed normal chow during prior and during the tests, whereas in other examples, the 12 week healthy SD rats were fed high fat diet (HFD) for eight weeks prior to the test and for the duration of the test.

Acylated Calcitonin Mimetics

The following Tables 1a and 1b set out the amino acid sequences of the acylated calcitonin mimetics that have been tested. As used therein:

1 acylation means K_{Ac}-(glutamic acid linker)-(C16 fatty acid [palmitate]);

2 acylation means K_{Ac}-(glutamic acid linker)-(C18 diacid [Octadecanedioic acid]);

3 acylation means K_{Ac}-(2×OEG amino acids linked together with a glutamic acid residue attached to N-terminus)-(C18 diacid [Octadecanedioic acid]).

4 acylation means KAc-(2×OEG amino acids linked together with a glutamic acid residue attached to N-terminus)-(C20 diacid [Eicosanedioic acid]).

5 acylation means KAc-(2×OEG amino acids linked together with a glutamic acid residue attached to N-terminus)-(C22 diacid [Docosanedioic acid]).

6 acylation means KAc-(2×OEG amino acids linked together with a glutamic acid residue attached to N-terminus)-(C16 diacid [Hexadecanedioic acid]).

7 acylation means KAc-(3×OEG amino acids linked together with a glutamic acid residue attached to N-terminus)-(C18 diacid [Octadecanedioic acid]).

8 acylation means KAc-(1×OEG amino acids linked together with a glutamic acid residue attached to N-terminus)-(C18 diacid [Octadecanedioic acid]).

9 acylation means KAc-(2×OEG amino acids linked together with a glutamic acid residue attached to N-terminus)-(C24 diacid [Tetracosanedioic acid]).

10 acylation means KAc-(2×OEG amino acids linked together with a glutamic acid residue attached to N-terminus)-(C26 diacid [Hexacosanedioic acid]).

11 acylation means KAc-(2×OEG amino acids linked together with a glutamic acid residue attached to N-terminus)-(C14 diacid [Tetradecanedioic acid]).

The tested calcitonin mimetics are based on the following core peptide sequences prior to modification:

CSNLSTCMLGRLSQDLHRLQTYPKTDVGANAP (KBP089)
CSNLSTC(AiB)LGRLSQDLHRLQTYPKTDVGANAP (KBP066)
CGNLSTC(AiB)LGRLTQDLNKFHTFPKTDVGANAP (KBP062)
CSNLSTCVLGKLSQELHKLQTYPRTDVGANAP (KBP042)
CSNLSTC(AiB)LGRLANFLVHSSNNFGAILPKTDVGANAP (KBP110)
CSNLSTCMLGRLSQELHRLQTYPKTDVGANAP (KBP021)

In Table 1b, the following additional nomenclature is also used:

| Acylated Amino Acid | KBP Name Modifier |
|---|---|
| 01 | A01 |
| 02 | A02 |
| 03 | A03 |
| . . . | . . . |
| XX | AXX |
| . . . | . . . |
| 31 | A31 |
| 32 | A32 |
| Type Acylation | Name Addition |
| C16 | .01 |
| C18 diacid | .02 |
| C18 diacid 2*OEG | .03 |
| C20 diacid 2*OEG | .04 |
| C22 diacid 2*OEG | .05 |
| C16 diacid 2*OEG | .06 |
| C18 diacid 3*OEG | .07 |
| C18 diacid 1*OEG | .08 |
| C24 diacid 2*OEG | .09 |
| C26 diacid 2*OEG | .10 |
| C14 diacid 2*OEG | .11 |

Thus, by way of example, the nomenclature KBP-066A11.03 indicates that the peptide consists of the KBP-066 core sequence, modified by substitution at the 11 position with a lysine residue with a C18 diacid 2*OEG acylation.

TABLE 1
Acylated calcitonin mimetics

| | N- | | | | | | | | | | | | | | | | | | | | | | | | | | | | | | | | | | | | | | c- |
|---|---|---|---|---|---|---|---|---|---|---|---|---|---|---|---|---|---|---|---|---|---|---|---|---|---|---|---|---|---|---|---|---|---|---|---|---|---|---|---|
| KBP | term | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 | 31 | 32 | 33 | 34 | 35 | 36 | 37 | term |
| 346 | Ac— | C | S | N | L | S | T | C | V | L | G | 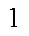 | L | S | Q | E | L | H | K | L | Q | T | Y | P | R | T | D | V | G | A | N | A | P | | | | | | —NH2 |
| 347 | Ac— | C | S | N | L | S | T | C | M | L | G | R | L | S | Q | D | L | H | 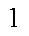 | L | Q | T | Y | P | K | T | D | V | G | A | N | A | P | | | | | | —NH2 |
| 363 | Ac— | C | S | N | L | S | T | C | M | L | G | 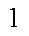 | L | S | Q | D | L | H | R | L | Q | T | Y | P | K | T | D | V | G | A | N | A | P | | | | | | —NH2 |
| [symbol] | Ac— | C | S | N | L | S | T | C | M | L | G | R | 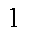 | S | Q | D | L | H | R | L | Q | T | Y | P | K | T | D | V | G | A | N | A | P | | | | | | —NH2 |
| [symbol] | Ac— | C | S | N | L | S | T | C | M | L | G | R | L | S | Q | D | 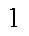 | H | R | L | Q | T | Y | P | K | T | D | V | G | A | N | A | P | | | | | | —NH2 |
| [symbol] | Ac— | C | S | N | L | S | T | C | M | 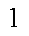 | G | R | L | S | Q | D | L | H | R | L | Q | T | Y | P | K | T | D | V | G | A | N | A | P | | | | | | —NH2 |
| [symbol] | Ac— | C | S | N | L | S | T | C | M | L | G | R | L | S | Q | D | L | H | R | L | Q | T | Y | P | K | T | D | V | G | A | N | A | P | K | | | | | 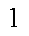 |
| [symbol] | Ac— | C | S | N | L | S | T | C | 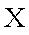 | 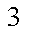 | G | R | L | S | Q | D | L | H | R | L | Q | T | Y | P | K | T | D | V | G | A | N | A | P | | | | | | —NH2 |
| [symbol] | Ac— | C | S | N | L | S | T | C | 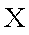 | 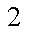 | G | R | L | S | Q | D | L | H | R | L | Q | T | Y | P | K | T | D | V | G | A | N | A | P | | | | | | —NH2 |
| [symbol] | Ac— | C | S | N | L | S | T | C | 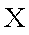 | L | G | | L | S | Q | D | L | H | R | L | Q | T | Y | P | K | T | D | V | G | A | N | A | P | | | | | | —NH2 |
| [symbol] | Ac— | C | S | N | L | S | T | C | 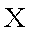 | L | G | 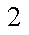 | L | S | Q | D | L | H | R | L | Q | T | Y | P | K | T | D | V | G | A | N | A | P | | | | | | —NH2 |
| [symbol] | Ac— | C | S | N | L | S | T | C | 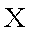 | L | G | R | L | S | Q | D | L | H | R | L | Q | T | Y | P | K | T | D | V | G | A | N | A | P | K | | | | | 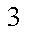 |
| [symbol] | Ac— | C | S | N | L | S | T | C | 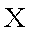 | L | G | R | L | S | Q | D | L | H | R | L | Q | T | Y | P | K | T | D | V | G | A | N | A | P | K | | | | | 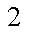 |
| [symbol] | Ac— | C | G | N | L | S | T | C | 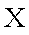 | 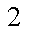 | G | R | L | T | Q | D | L | N | K | F | H | T | F | P | K | T | D | V | G | A | N | A | P | | | | | | —NH2 |
| [symbol] | Ac— | C | G | N | L | S | T | C | 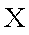 | 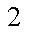 | G | R | L | T | Q | D | L | N | K | F | H | T | F | P | K | T | D | V | G | A | N | A | P | | | | | | —NH2 |
| 363 | Ac— | C | G | N | L | S | T | C | 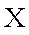 | L | G | | L | T | Q | D | L | N | K | F | H | T | F | P | K | T | D | V | G | A | N | A | P | | | | | | —NH2 |
| [symbol] | Ac— | C | G | N | L | S | T | C | 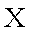 | L | G | 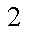 | L | T | Q | D | L | N | K | F | H | T | F | P | K | T | D | V | G | A | N | A | P | | | | | | —NH2 |
| 286 | Ac— | C | G | N | L | S | T | C | 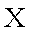 | L | G | R | L | T | Q | D | L | N | K | P | H | T | F | P | K | T | D | V | G | A | N | A | P | K | | | | | 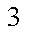 |
| 366 | Ac— | C | G | N | L | S | T | C | 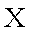 | L | G | R | L | T | Q | D | L | N | K | F | H | T | F | P | K | T | D | V | G | A | N | A | P | K | | | | | 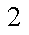 |
| [symbol] | Ac— | C | S | N | L | S | T | C | 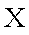 | 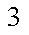 | G | R | L | A | N | F | L | V | H | S | S | N | N | F | G | A | I | L | P | K | T | D | V | G | A | N | A | P | —NH2 |
| [symbol] | Ac— | C | S | N | L | S | T | C | 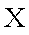 | 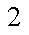 | G | R | L | A | N | F | L | V | H | S | S | N | N | F | G | A | I | L | P | K | T | D | V | G | A | N | A | P | —NH2 |
| 368 | Ac— | C | S | N | L | S | T | C | 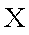 | L | G | 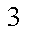 | L | A | N | F | L | V | H | S | S | N | N | F | G | A | I | L | P | K | T | D | V | G | A | N | A | P | —NH2 |
| 369 | Ac— | C | S | N | L | S | T | C | 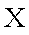 | L | G | 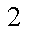 | L | A | N | F | L | V | H | S | S | N | N | F | G | A | I | L | P | K | T | D | V | G | A | N | A | P | —NH2 |
| STD | Ac— | C | S | N | L | S | T | C | 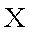 | L | G | R | L | A | N | F | L | V | H | S | S | N | N | F | G | A | I | L | P | K | T | D | V | G | A | N | A | P | 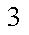 |
| [symbol] | Ac— | C | S | N | L | S | T | C | 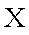 | L | G | R | L | A | N | F | L | V | H | S | S | N | N | F | G | A | I | L | P | K | T | D | V | G | A | N | A | P | 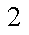 |
| 372 | Ac— | C | S | N | L | S | T | C | V | L | G | 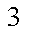 | L | S | Q | E | L | H | K | L | Q | T | Y | P | R | T | D | V | G | A | N | A | P | | | | | | —NH2 |
| 373 | Ac— | C | S | N | L | S | T | C | V | 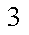 | G | K | L | S | Q | E | L | H | K | L | Q | T | Y | P | R | T | D | V | G | A | N | A | P | | | | | | —NH2 |
| 374 | Ac— | C | S | N | L | S | T | C | V | L | G | K | L | S | Q | E | L | H | K | L | Q | T | Y | P | R | T | D | V | G | A | N | A | P | K | | | | | 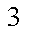 |
| 375 | Ac— | C | S | N | L | S | T | C | M | 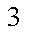 | G | R | L | S | Q | D | L | H | R | L | Q | T | Y | P | K | T | D | V | G | A | N | A | F | | | | | | —NH2 |
| 376 | Ac— | C | S | N | L | S | T | C | M | L | G | 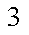 | L | S | Q | D | L | H | R | L | Q | T | Y | P | K | T | D | V | G | A | N | A | P | | | | | | —NH2 |
| 377 | Ac— | C | S | N | L | S | T | C | M | L | G | R | L | S | Q | D | L | H | R | L | Q | T | Y | P | K | T | D | V | G | A | N | A | P | K | | | | | 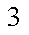 |
| 378 | Ac— | C | A | S | L | S | T | C | V | 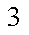 | G | K | L | S | Q | D | L | H | K | L | Q | T | F | P | K | T | D | V | G | A | N | A | P | | | | | | —NH2 |
| 379 | Ac— | C | A | S | L | S | T | C | V | L | G | 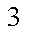 | L | S | Q | D | L | H | K | L | Q | T | F | P | K | T | D | V | G | A | N | A | P | | | | | | —NH2 |
| 380 | Ac— | C | A | S | L | S | T | C | V | L | G | K | L | S | Q | D | L | H | K | L | Q | T | F | P | K | T | D | V | G | A | N | A | P | K | | | | | 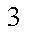 |

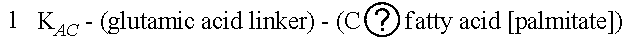

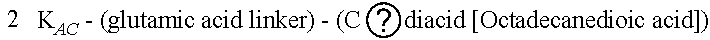

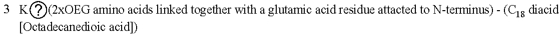

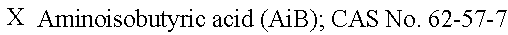

[symbol]

indicates data missing or illegible when filed

TABLE 1b
Acylated Calcitonin Mimetics

| | | | N- | | | | | | | | | | | | | | | | | |
|---|---|---|---|---|---|---|---|---|---|---|---|---|---|---|---|---|---|---|---|---|
| KBP | Core peptide | Acylation | term | 1 | 2 | 3 | 4 | 5 | 6 | 7 | 8 | 9 | 10 | 11 | 12 | 13 | 14 | 15 | 16 | 17 |
| [symbol] | KBP-065 | A11.04 | Ac— | C | S | N | L | S | T | C | 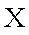 | L | G | 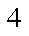 | L | S | Q | D | L | H |
| 381 | KBP-066 | A11.05 | Ac— | C | S | N | L | S | T | C | 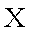 | L | G | 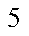 | L | S | Q | D | L | H |
| [symbol] | KBP-066 | A11.06 | Ac— | C | S | N | L | S | T | C | 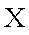 | L | G | 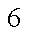 | L | S | Q | D | L | H |
| 305 | KBP- [symbol] | A11.07 | Ac— | C | S | N | L | S | T | C | 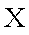 | L | G | 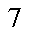 | L | S | Q | D | L | H |
| [symbol] | KGP-065 | A11.08 | Ac— | C | S | N | L | S | T | C | 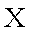 | L | G | 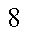 | L | S | Q | D | L | H |
| 307 | KBP-066 | A11.09 | Ac— | C | S | N | L | S | T | C | 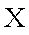 | L | G | 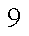 | L | S | Q | D | L | H |
| [symbol] | KBP-064 | A11.16 | Ac— | C | S | N | L | S | T | C | 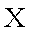 | L | G | 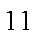 | L | S | Q | D | L | H |
| 305 | KBP-066 | A11.13 | Ac— | C | S | N | L | S | T | C | 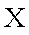 | L | G | 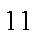 | L | S | Q | D | L | H |
| 354 | KBP-066 | A09.03 | Ac— | C | S | N | L | S | T | C | 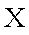 | 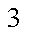 | G | R | L | S | Q | D | L | H |
| 356 | KBP-066 | A11.03 | Ac— | C | S | N | L | S | T | C | 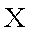 | L | G | 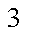 | L | S | Q | D | L | H |
| [symbol] | KBP-068 | A12.03 | Ac— | C | S | N | L | S | T | C | 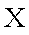 | L | G | R | 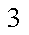 | S | Q | D | L | H |
| 387 | KBP-064 | A16.03 | Ac— | C | S | N | L | S | T | C | 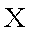 | L | G | R | L | S | Q | D | 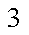 | H |
| 348 | KBP-064 | A16.03 | Ac— | C | S | N | L | S | T | C | 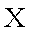 | L | G | R | L | S | Q | D | L | H |
| 399 | KBP-064 | A19.03 | Ac— | C | S | N | L | S | T | C | 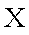 | L | G | R | L | S | Q | D | L | H |
| 399 | KBP-066 | A19.05 | Ac— | C | S | N | L | S | T | C | 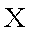 | L | G | R | L | S | Q | D | L | H |
| 390 | KBP-064 | A24.03 | Ac— | C | S | N | L | S | T | C | 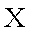 | L | G | R | L | S | Q | D | L | H |
| 358 | KBP-066 | A32.03 | Ac— | C | S | N | L | S | T | C | 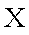 | L | G | R | L | S | Q | D | L | H |
| 312 | KBP-021 | A09.03 | Ac— | C | S | N | L | S | T | C | M | 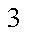 | G | R | L | S | Q | 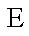 | L | H |
| 391 | KBP-021 | A11.03 | Ac— | C | S | N | L | S | T | C | M | L | G | 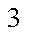 | L | S | Q | 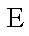 | L | H |
| 393 | KBP-035 | A11.06 | Ac— | C | S | N | L | S | T | C | M | L | C | 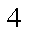 | L | S | Q | 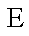 | L | H |
| 394 | KBP-021 | A11.06 | Ac— | C | S | N | L | S | T | C | M | L | G | 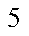 | L | S | Q | 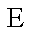 | L | H |
| [symbol] | KBP-021 | A11.03 | Ac— | C | S | N | L | S | T | C | M | L | G | R | 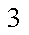 | S | Q | 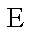 | L | H |
| 314 | KBP-021 | A16.03 | Ac— | C | S | N | L | S | T | C | M | L | G | R | L | S | Q | 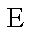 | 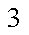 | H |
| 315 | KBP-021 | A19.03 | Ac— | C | S | N | L | S | T | C | M | L | G | R | L | S | Q | 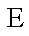 | L | H |
| 316 | KBP-021 | A19.03 | Ac— | C | S | N | L | S | T | C | M | L | G | R | L | S | Q | 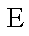 | L | H |
| 395 | KBP-021 | A19.05 | Ac— | C | S | N | L | S | T | C | M | L | G | R | L | S | Q | 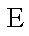 | L | H |
| 217 | KBP-021 | A24.03 | Ac— | C | S | N | L | S | T | C | M | L | G | R | L | S | Q | 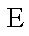 | L | H |
| 318 | KBP-021 | A32.03 | Ac— | C | S | N | L | S | T | C | M | L | G | R | L | S | Q | 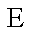 | L | H |
| | | | KBP | 18 | 19 | 20 | 21 | 22 | 23 | 24 | 25 | 26 | 27 | 28 | 29 | 30 | 31 | 32 | 33 | C-term |
| | | | [symbol] | R | L | Q | T | Y | P | K | T | D | V | G | A | N | A | P | | —NH2 |
| | | | 381 | R | L | Q | T | Y | P | K | T | D | V | G | A | N | A | P | | —NH2 |
| | | | [symbol] | R | L | Q | T | Y | P | K | T | D | V | G | A | N | A | P | | —NH2 |
| | | | 305 | R | L | Q | T | Y | P | K | T | D | V | G | A | N | A | P | | —NH2 |
| | | | [symbol] | R | L | Q | T | Y | P | K | T | D | V | G | A | N | A | P | | —NH2 |
| | | | 307 | R | L | Q | T | Y | P | K | T | D | V | G | A | N | A | P | | —NH2 |
| | | | [symbol] | R | L | Q | T | Y | P | K | T | D | V | G | A | N | A | P | | —NH2 |
| | | | 305 | R | L | Q | T | Y | P | K | T | D | V | G | A | N | A | P | | —NH2 |
| | | | 354 | R | L | Q | T | Y | P | K | T | D | V | G | A | N | A | P | | —NH2 |
| | | | 356 | R | L | Q | T | Y | P | K | T | D | V | G | A | N | A | P | | —NH2 |
| | | | [symbol] | R | L | Q | T | Y | P | K | T | D | V | G | A | N | A | P | | —NH2 |
| | | | 387 | R | L | Q | T | Y | P | K | T | D | V | G | A | N | A | P | | —NH2 |
| | | | 348 | 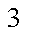 | L | Q | T | Y | P | K | T | D | V | G | A | N | A | P | | —NH2 |
| | | | 399 | R | 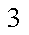 | Q | T | Y | P | K | T | D | V | G | A | N | A | P | | —NH2 |
| | | | 399 | R | 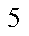 | Q | T | Y | P | K | T | D | V | G | A | N | A | P | | —NH2 |
| | | | 390 | R | L | Q | T | Y | P | 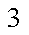 | T | D | V | G | A | N | A | P | | —NH2 |
| | | | 358 | R | L | Q | T | Y | P | K | T | D | V | G | A | N | A | P | K | 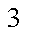 |
| | | | 312 | R | L | Q | T | Y | P | K | T | D | V | G | A | N | A | P | | —NH2 |
| | | | 391 | R | L | Q | T | Y | P | K | T | D | V | G | A | N | A | P | | —NH2 |
| | | | 393 | R | L | Q | T | Y | P | K | T | D | V | C | A | N | A | P | | —NH2 |
| | | | 394 | R | L | Q | T | Y | P | K | T | D | V | G | A | N | A | P | | —NH2 |
| | | | [symbol] | R | L | Q | T | Y | P | K | T | D | V | G | A | N | A | P | | —NH2 |
| | | | 314 | R | L | Q | T | Y | P | K | T | D | V | G | A | N | A | P | | —NH2 |
| | | | 315 | 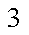 | L | Q | T | Y | P | K | T | D | V | G | A | N | A | P | | —NH2 |
| | | | 316 | R | 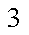 | Q | T | Y | P | K | T | D | V | G | A | N | A | P | | —NH2 |
| | | | 395 | R | 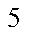 | Q | T | Y | P | K | T | D | V | G | A | N | A | P | | —NH2 |
| | | | 217 | R | L | Q | T | Y | P | 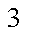 | T | D | V | G | A | N | A | P | | —NH2 |
| | | | 318 | R | L | Q | T | Y | P | K | T | D | V | G | A | N | A | P | K | 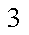 |

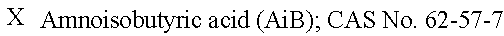

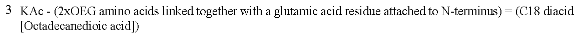

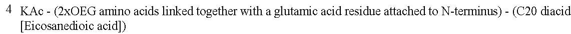

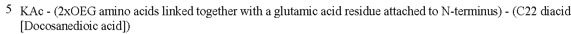

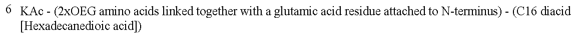

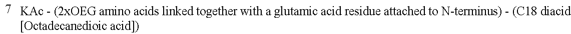

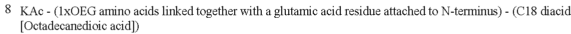

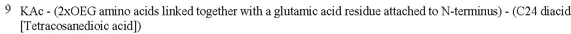

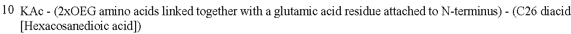

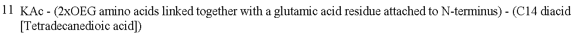

[symbol]

indicates data missing or illegible when filed

The various acylations have the following chemical structures:

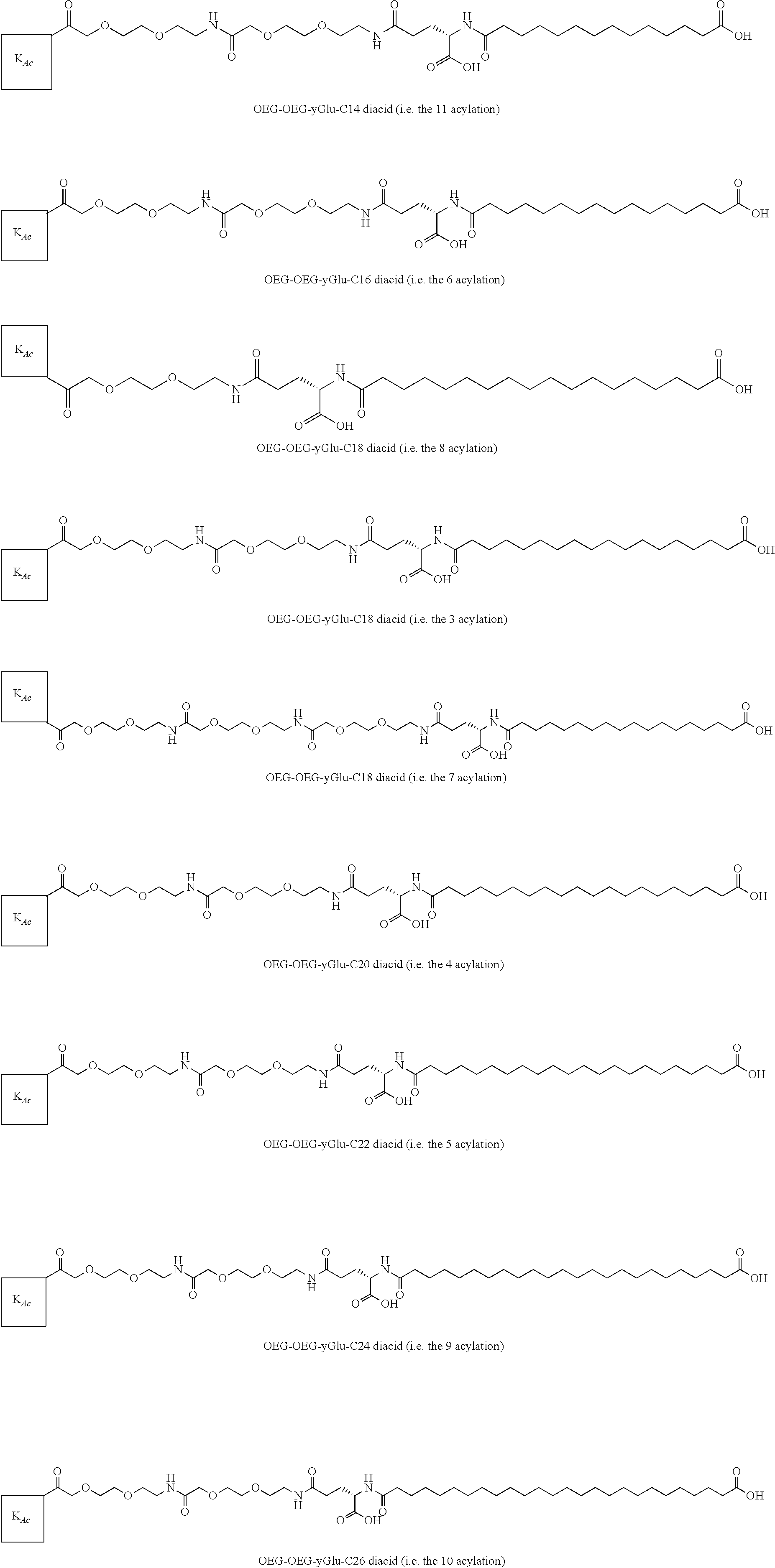

### Initial Acylation Studies (Examples 1-5)

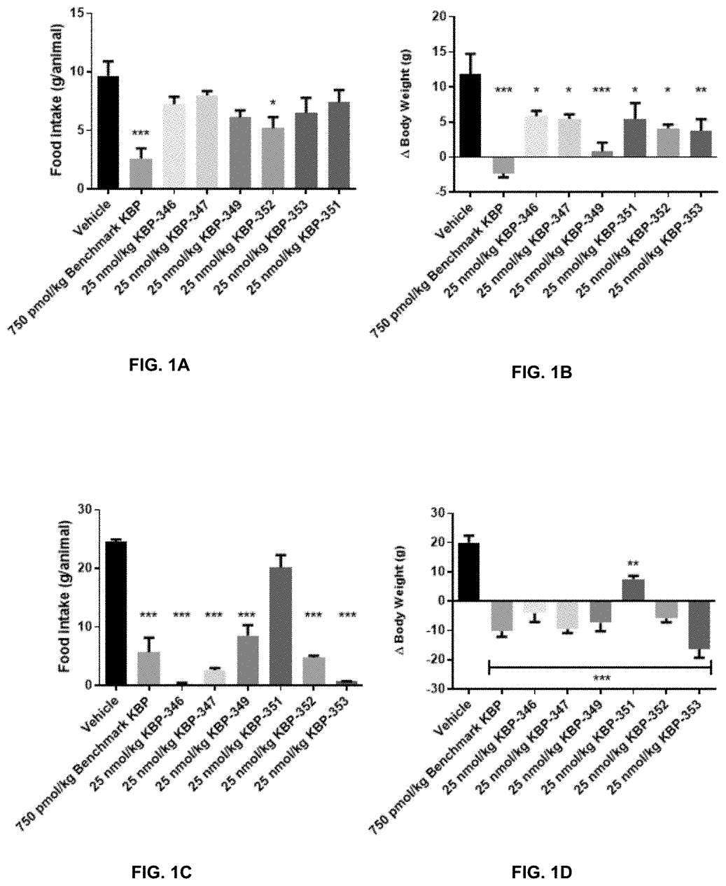

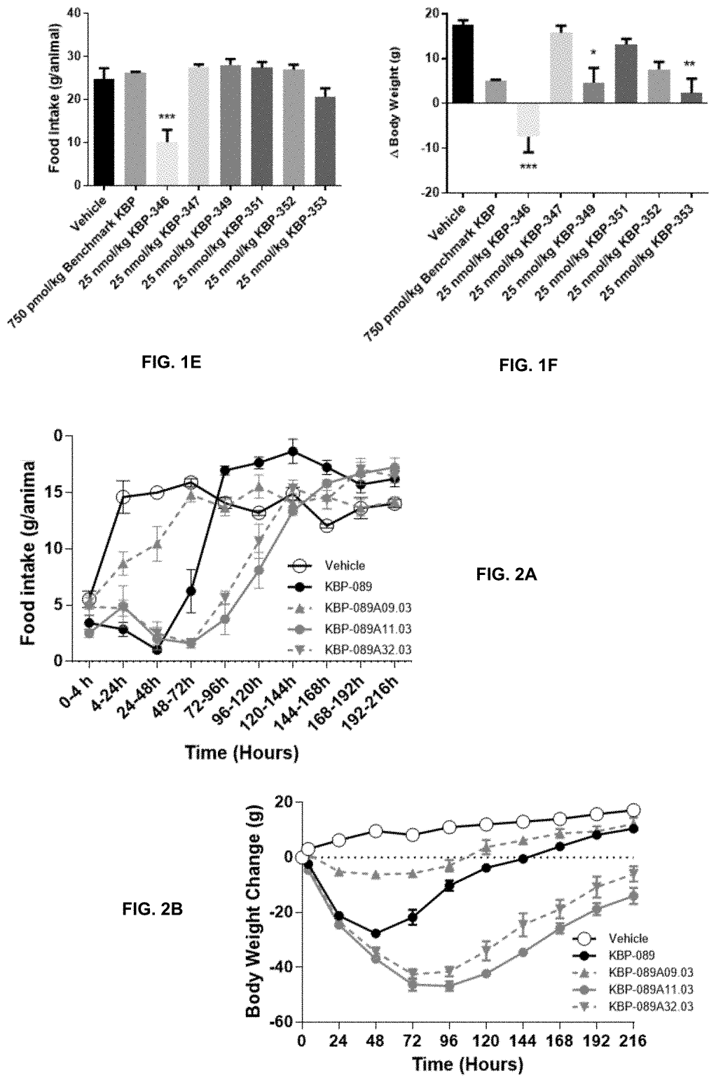

### Example 1 (FIGS. 1 & 5)

Single dose comparative effect of 1 acylated variants at different positions (9 position “A09”, 11 position “A11”, 16 position “A16”, 18 position “A18”, and 32 position “A32”) to a non-acylated Benchmark peptide (KBP-089) on food intake and body weight in 12 week lean SD rats.

| KBP | Core | Position/Acylation |
|---|---|---|
| KBP-346 | KBP-042 | A11/1 acylation |
| KBP-347 | KBP-089 | A18/1 acylation |
| KBP-349 | KBP-089 | A11/1 acylation |
| KBP-350 | KBP-089 | A12/1 acylation |
| KBP-351 | KBP-089 | A16/1 acylation |
| KBP-352 | KBP-089 | A9/1 acylation |
| KBP-353 | KBP-089 | A32/1 acylation |

Rats were single caged four days prior to the test. Rats were randomized by weight into six groups (Vehicle (0.9% NaCl), KBPs (doses: 25 nmol/kg (\hat{ }100 μg/kg)). They were fasted overnight and then treated with a single dose of peptide or vehicle in the morning using subcutaneous administration. Food intake was monitored in the following intervals (0-4 hours, 4-24 hours, 24-48 hours). Body weight was measured at baseline and at 24 hours and 48 hours post s.c injection.

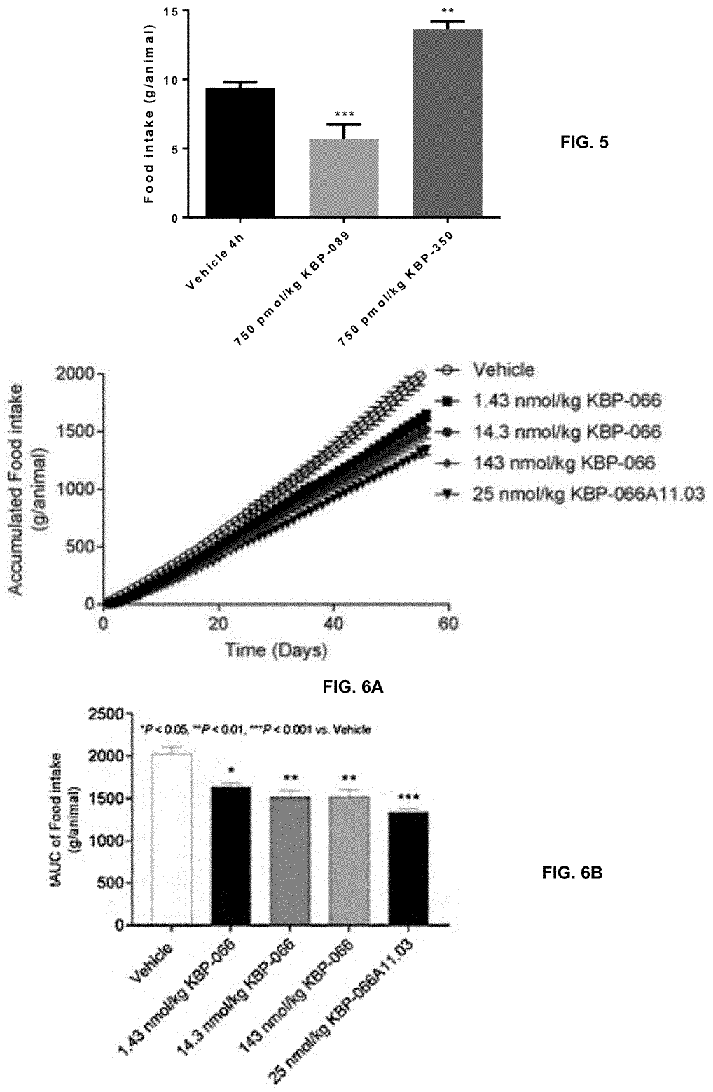

Acylation at positions “A09”, “A11” and “A32” with 1 acylation produced a protracted in vivo response (FIG. 1) that merited further testing (see below). Positions 12 (FIG. 5), 16, and 18 returned an unacceptable result and were not advanced into further experiments.

### Example 2: β-Arrestin Assay

PathHunter β-arrestin GPCR assays are whole cell, functional assays that directly measure the ability of a ligand to activate a GPCR by detecting the interaction of β-arrestin with the activated GPCR. Because β-arrestin recruitment is independent of G-protein signaling, these assays offer a powerful and universal screening and profiling platform that can be used for virtually any Gi-, Gs, or Gq-coupled receptor.

In this system, the GPCR is fused in frame with the small enzyme fragment ProLink™ and co-expressed in cells stably expressing a fusion protein of β-arrestin and the larger, N-terminal deletion mutant of β-gal (called enzyme acceptor or EA). Activation of the GPCR stimulates binding of β-arrestin to the ProLink-tagged GPCR and forces complementation of the two enzyme fragments, resulting in the formation of an active β-gal enzyme. This interaction leads to an increase in enzyme activity that can be measured using chemiluminescent PathHunter® Detection Reagents.

In independent bioassays, CTR and AMY-R cells were treated at the indicated time points with increasing doses of KBPs identified in Tables 2 and 3 below (100, 20, 4, 0.8, 0.16, 0.032 nM and vehicle). The assay was performed in white 384 well plates (Greiner Bio-One, 784080). Cells were seeded 2500 cells per well in 10 μL cell-type specific medium the day prior to the experiment. To quantify the GPCR-mediated β-arrestin recruitment the Pathhunter™ Detection Kit (93-0001, DiscoverX) was used and assay performed accordingly to the manufacturer's instructions.

The prolonged/protracted response was conducted using the calcitonin receptor (CTR): U2OS-CALCR from DiscoveRx (Cat. No.: 93-0566C3) cell line, and as opposed to the classical three hour output, β-arrestin accumulation was conducted over 3, 6, 24, 48 or 72 hour and then assayed and analyzed. Table 2 (2 acylation) and Table 3 (3 acylation) set out the results of the β-arrestin study.

TABLE 2
β-arrestin study for the 2 acylation (K_{Ac}-(glutamic acid linker)-(C18 diacid))

| | | | Compound | | | | | |
|---|---|---|---|---|---|---|---|---|
| | | | U2OS | | U2OS | | CHO-K1 | |
| | | | (CTR) | | (CTR) | | (AMY-R) | |
| | | | Acylated KBPs 3 Acylation | | | | | |
| | | | | | β-arrestin | | | |
| | | | β-arrestin | | Prolonged | | β-arrestin | |
| | | | Fold | | CTR response | | Fold | |
| | | | Recruitment | | (10 nM) | | Recruitment | |
| | Core | Acylation | EC50 values | | tAUC value | | EC50 values | |
| NO | Sequence | Position/Type | (10⁻⁹ M) | | 0-72 h | | (10⁻⁰⁹ M) | |
| KBP-355 | KBP-066 | A09/2 | 31.2 ± 4.4 | (3) | 147 ± 004 | (2) | 509 ± 695 | (3) |
| KBP-357 | KBP-066 | A11/2 | 9.2 ± 1.0 | (3) | 1576 ± 171 | (2) | 11.4 ± 5.8 | (3) |
| KBP-359 | KBP-066 | A32/2 | 40.8 ± 7.2 | (3) | 1438 ± 003 | (2) | 96.5 ± 65 | (3) |
| KBP-361 | KBP-062 | A09/2 | 127.5 ± 45 | (3) | 136 ± 007 | (2) | 18.4 | (1) |
| KBP-363 | KBP-062 | A11/2 | 10.9 ± 7.0 | (3) | 1581 ± 066 | (2) | 36.6 ± 31 | (2) |
| KBP-365 | KBP-062 | A32/2 | 34.6 ± 6.8 | (3) | 1282 ± 034 | (2) | 51.9 ± 1.6 | (2) |
| KBP-367 | KBP-110 | A09/2 | >1000 | (3) | 095 ± 020 | (3) | >1000 | (3) |
| KBP-369 | KBP-110 | A11/2 | 182 ± 1.2 | (3) | 537 ± 073 | (3) | 230 ± 4.3 | (3) |
| KBP-371 | KBP-110 | A32/2 | >1000 | (3) | 109 ± 001 | (3) | >1000 | (3) |

Table 2: In vitro peptide screening characteristics.
AX/2 means position X with a 2 acylation, e.g. A09/2 means acylation at the 9 position with the 2 acylation.

TABLE 3
β-arrestin study for the 3 acylation (K_{Ac}-(2xOEG amino acids linked together with
a glutamic acid residue attached to N-terminus)-(C18 diacid [Octadecanedioic acid])

| | | | Compound | | | | | | | |
|---|---|---|---|---|---|---|---|---|---|---|
| | | | U2OS | | U2OS | | CHO-K1 | | Food | |
| | | | (CTR) | | (CTR) | | (AMY-R) | | Intake | |
| | | | Acylated KBPs 2 Acylation | | | | | | | |
| | | | | | β-arrestin | | | | | |
| | | | β-arrestin | | Prolonged | | β-arrestin | | | |
| | | | Fold | | CTR response | | Fold | | ΔFOOD | |
| | | | Recruitment | | (10 nM) | | Recruitment | | Sustained | |
| | | | EC50 | | tAUC | | EC50 | | Attenuation | |
| | Core | Acylation | values | | value | | values | | (36 nmol/kg) | |
| NO | Sequence | Type | (10⁻⁹ M) | | 0-72 h | | (10⁻⁰⁹ M) | | Hours (h) | |
| KBP-354 | KBP-066 | A09/3 | 4.7 ± 0.6 | (3) | 260 ± 019 | (2) | 93.0 ± 26 | (3) | 4 | h |
| KBP-356 | KBP-066 | A11/3 | 8.5 ± 0.8 | (3) | 2512 ± 295 | (2) | 12.0 ± 4.0 | (3) | 96 | h |
| KBP-358 | KBP-066 | A32/3 | 44.2 ± 5.7 | (3) | 1460 ± 202 | (2) | 98.6 ± 53 | (3) | 72 | h |
| KBP-360 | KBP-062 | A09/3 | 45.2 ± 9.4 | (3) | 182 ± 006 | (2) | 83.9 ± 42 | (3) | 4 | h |
| KBP-362 | KBP-062 | A11/3 | 13.2 ± 9.6 | (3) | 1784 ± 330 | (2) | 14.5 ± 0.2 | (2) | 72 | h |
| KBP-364 | KBP-062 | A32/3 | 53.3 ± 8.6 | (3) | 1322 ± 035 | (2) | 106 ± 32 | (2) | 48 | h |
| KBP-366 | KBP-110 | A09/3 | >1000 | (3) | 084 ± 007 | (3) | >1000 | (3) | 4 | h |
| KBP-368 | KBP-110 | A11/3 | 193 ± 2.9 | (3) | 827 ± 140 | (3) | 166 ± 43 | (3) | 72 | h |
| KBP-370 | KBP-110 | A32/3 | 473 ± 34 | (3) | 635 ± 077 | (3) | >1000 | (3) | 4 | h |
| KBP-373 | KBP-042 | A09/3 | 96.5 ± 17 | (3) | 337 | (1) | 263 ± 7.3 | (3) | 4 | h |
| KBP-372 | KBP-042 | A11/3 | 7.8 ± 2.5 | (3) | 1304 ± 238 | (3) | 45.6 ± 12 | (3) | 96 | h |
| KBP-374 | KBP-042 | A32/3 | 49.2 ± 6.4 | (3) | 1073 | (1) | 151 ± 15 | (4) | 72 | h |
| KBP-375 | KBP-089 | A09/3 | 56.3 ± 20 | (3) | 624 | (1) | 232 ± 27 | (4) | 4 | h |
| KBP-376 | KBP-089 | A11/3 | 14.7 ± 2.7 | (3) | 1395 | (1) | 25.0 ± 2.2 | (4) | 96 | h |
| KBP-377 | KBP-089 | A32/3 | 66.0 ± 36 | (3) | 1403 | (1) | 73.1 ± 7.4 | (4) | 72 | h |

Table 3: In vitro peptide screening characteristics
AX/3 means position X with a 3 acylation, e.g. A09/3 means acylation at the 9 position with the 3 acylation.

The β-arrestin studies indicated the following:

1) Potency of the acylations in terms of the acylation position on the peptide is as follows: A11>A32>A09.

2) The 2 or 3 acylation at the 11 position (A11) is the generally far superior acylation/position combination for every peptide core in terms of activing the calcitonin receptor (CTR), the amylin receptor (AMY-R), prolonged CTR response, and suppressing food intake.

3) Acylated KBPs with different cores demonstrate similar potency and patterns in vitro when modified with identical acylations.

### Example 3 (FIG. 2)

Single dose comparative effect of A09 (KBP375), A11 (KBP376) and A32 (KBP377) 3 acylated variants of KBP089 with the non-acylated Benchmark KBP089 on food intake and body weight in 20 week HFD SD rats.

| KBP | Core | Annotation | Position/Acylation |
|---|---|---|---|
| KBP-375 | KBP-089 | KBP-089A09.03 | A9/3 acylation |
| KBP-376 | KBP-089 | KBP-089A11.03 | A11/3 acylation |
| KBP-377 | KBP-089 | KBP-089A32.03 | A32/3 acylation |

Rats were single caged four days prior to the test. Rats were randomized by weight into eleven groups (Vehicle (0.9% NaCl), KBPs (doses: 36 nmol/kg (150-157 μg/kg)). They were fasted overnight and then treated with a single dose of peptide or vehicle in the morning using subcutaneous administration. Food intake was monitored in the following intervals (0-4 hours, 4-24 hours, 24-48 hours . . . 144-168 hours). Body weight was measured at baseline and every 24 hours post s.c injection.

The animal model studies confirmed the results of the β-arrestin study and demonstrated improved efficacy vis-à-vis the naked peptide:

1) A11>A32>A09 in terms of benefit of acylation position using KBP-089 as core peptide.

2) 2 acylation and 3 acylation are far superior to non-acylated KBP-089 at the dose given in terms of protracted in vivo activity and efficacy.

The animal model study also showed that acylating at the 9 position reduced the potency of the peptide when compared to the naked peptide, thereby ruling out the 9 position as a position of interest in further studies.

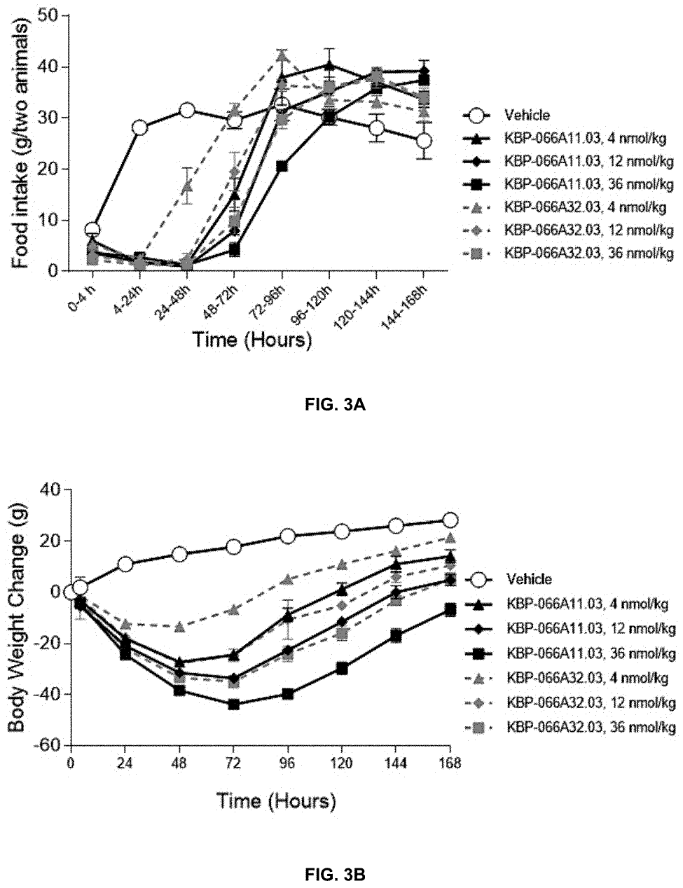

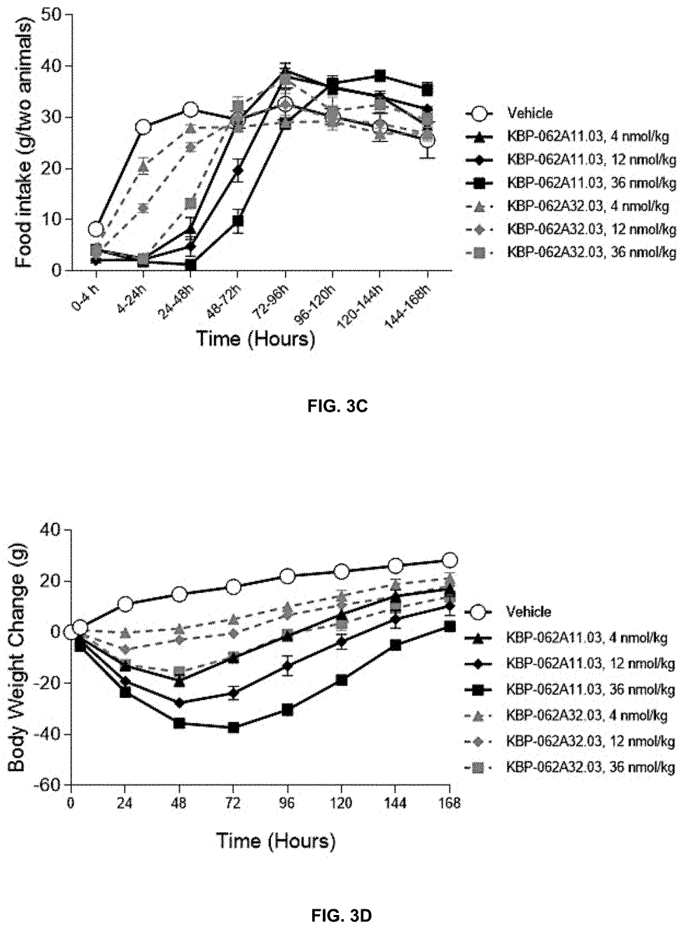

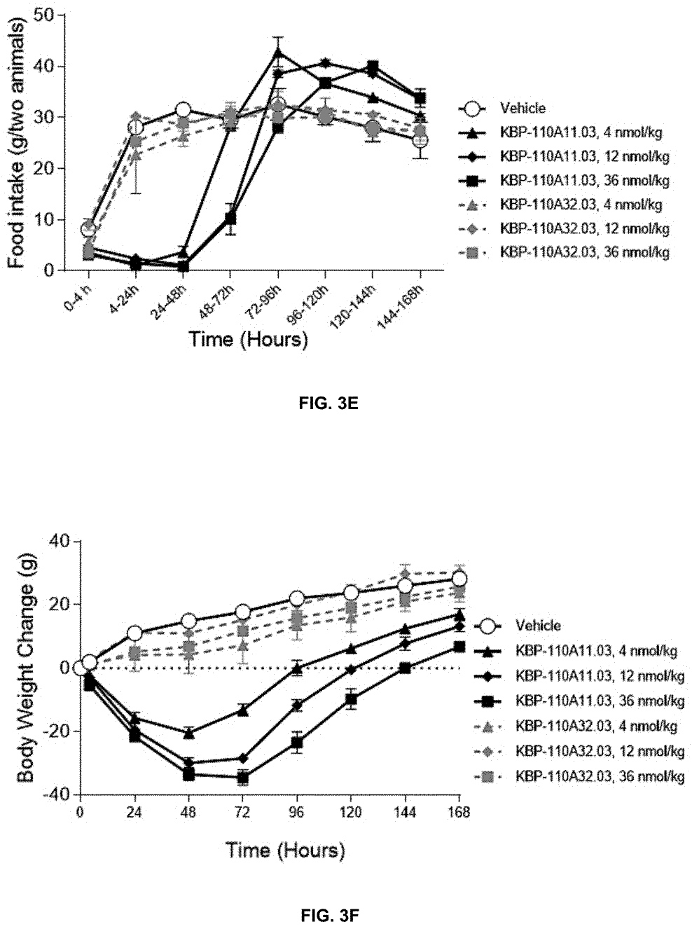

### Example 4 (FIG. 3)

Single dose comparative effect of A11 and A32 3 acylated variants with different peptide core to the respective non-acylated Benchmark KBP (KBP-066, KBP-062 and 7KBP-110) on food intake and body weight in 20 week HFD SD rats.

| KBP | Core | Annotation | Position/Acylation |
|---|---|---|---|
| KBP-356 | KBP-066 | KBP-066A11.03 | A11/3 acylation |
| KBP-358 | KBP-066 | KBP-066A32.03 | A32/3 acylation |
| KBP-362 | KBP-062 | KBP-062A11.03 | A11/3 acylation |
| KBP-364 | KBP-062 | KBP-062A32.03 | A32/3 acylation |
| KBP-368 | KBP-110 | KBP-110A11.03 | A11/3acylation |
| KBP-370 | KBP-110 | KBP-110A32.03 | A32/3 acylation |

Rats were single caged four days prior to the test. Rats were randomized by weight into eleven groups (Vehicle (0.9% NaCl), KBPs (doses: 4 nmol/kg (\hat{ }17 μg/kg), 12 nmol/kg (\hat{ }50 μg/kg) or 36 nmol/kg (\hat{ }50 μg/kg)). They were fasted overnight and then treated with a single dose of peptide or vehicle in the morning using subcutaneous administration. Food intake was monitored in the following intervals (0-4 hours, 4-24 hours, 24-48 hours . . . 144-168 hours). Body weight was measured at baseline and every 24 hours post s.c injection.

The results are as follows:

1) The peptide core does not affect the improvement observed by acylating at the 11 or 32 positions.

2) A11 is a better acylation site than A32.

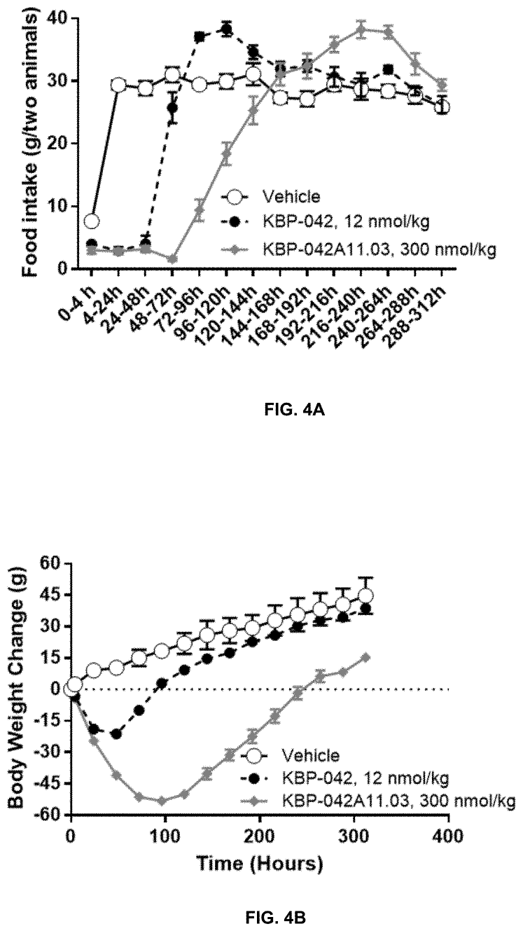

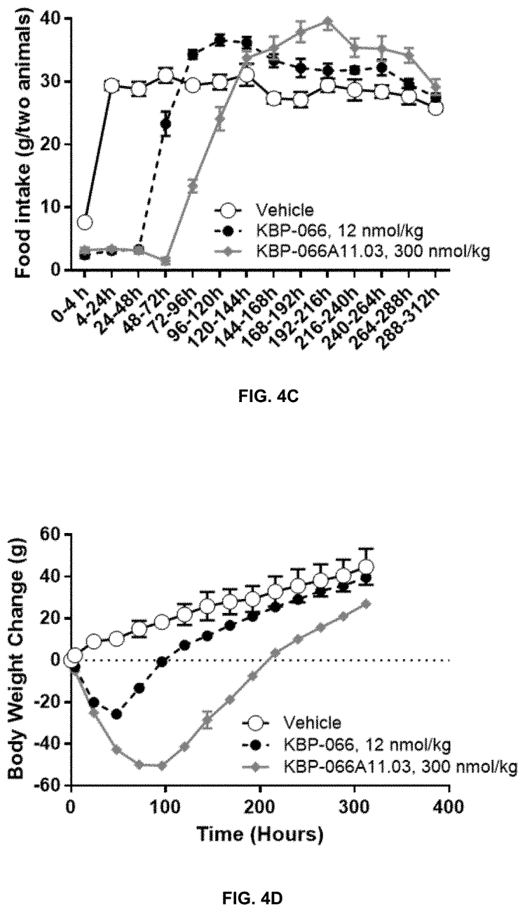

### Example 5 (FIG. 4)

Single high dose effect of A11/3 acylated variants of KBP-042 and KBP-066 on food intake and body weight in 20 week HFD SD rats. Rats were single caged four days prior to the test. Rats were randomized by weight into eleven groups (Vehicle (0.9% NaCl), KBPs (doses: 300 nmol/kg (\hat{ }1000 μg/kg)).

| KBP | Core | Annotation | Position/Acylation |
|---|---|---|---|
| KBP-372 | KBP-042 | KBP-042A11.03 | A11/3 acylation |
| KBP-356 | KBP-066 | KBP-066A11.03 | A11/3 acylation |

The rats were fasted overnight and then treated with a single dose of peptide or vehicle in the morning using subcutaneous administration. Food intake was monitored in the following intervals (0-4 hours, 4-24 hours, 24-48 hours . . . 188-312 hours). Body weight was measured at baseline and every 24 hours post s.c injection.

The high dose test using KBP356 and KBP372 demonstrated a superior protracted in vivo efficacy that lasted for days. These acylated peptides are therefore clear candidates for development of a once-weekly peptide therapeutic.

### Example 6 (FIGS. 6-10)

Further work was performed on compound KBP-356 (KBP-066A11.03), which comprises an AiB residue at the 8 position and the preferred acylation at the 11 position of the peptide.

A chronic study was performed in male ZDF rats. (obese homozygous recessive (fa/fa) strain: 370) (Charles River, USA). Rats were delivered 5 weeks of age. The rats were housed 2-3 per cage.

### Chronic Treatment of Male ZDF Rats:

Rats were delivered to the animal facility of Nordic Bioscience at five weeks of age (DAY −6). Rats were acclimatized for three days. HbA1c and BW was registered (DAY −3). Rats were randomized based on HbA1c (primarily) and BW (secondly) at day 4. The study was initiated at DAY 1.

### Dosage Concentrations and Frequency

Animals were dosed once daily with KBP-066 or saline (vehicle). Dosing with KBP-066A11.03 was performed once every third day. Dosing was administered subcutaneously (SC) around noon.

Saline: Dosage volume was 1 mL/kg.
KBP-066: Dosage volume was 1 mL/kg, Dosage concentration was 5, 50 or 500 μg/kg, and compound concentration was 5, 50 or 500 mg/L. The dose equivalent in nmol/kg is 1.43, 14.3 and 143 nmol/kg, respectively.
KBP-066A11.03: Dosage volume was 1 mL/kg, dosage concentration was 25 nmol/kg, and compound concentration was 25 mmol/L. The dose equivalent in μg/kg is 104 μg/kg.

Treatment groups in nmol/kg

| | | | Dosing | Dosing | | Compound | | Admin. | |
|---|---|---|---|---|---|---|---|---|---|
| Intervention | | Compound | volume | conc. | | conc. | | route | n |
| Vehicle | | Saline | 1 mL/kg | NA | | NA | | SC. | 10 |
| 1.43 | nmol/kg | KBP-066 | 1 mL/kg | 1.43 | nmol/kg | 1.43 | μmol/L | SC. | 10 |
| 14.3 | nmol/kg | KBP-066 | 1 mL/kg | 14.3 | nmol/kg | 14.3 | μmol/L | SC. | 10 |
| 143 | nmol/kg | KBP-066 | 1 mL/kg | 143 | nmol/kg | 143 | μmol/L | SC. | 10 |
| 25.0 | nmol/kg | KBP-066A11.03 | 1 mL/kg | 25.0 | nmol/kg | 25.0 | μmol/L | SC. | 10 |

Treatment groups in μg/kg

| | | | Dosing | Dosing | | Compound | | Admin. | |
|---|---|---|---|---|---|---|---|---|---|
| Intervention | | Compound | volume | conc. | | conc. | | route | n |
| Vehicle | | Saline | 1 mL/kg | NA | | NA | | SC. | 10 |
| 5 | μg/kg | KB P-066 | 1 mL/kg | 5 | μg/kg | 5 | mg/L | SC. | 10 |
| 50 | μg/kg | KB P-066 | 1 mL/kg | 50 | μg/kg | 50 | mg/L | SC. | 10 |
| 500 | μg/kg | KB P-066 | 1 mL/kg | 500 | μg/kg | 500 | mg/L | SC. | 10 |
| 104 | μg/kg | KBP-066A11.03 | 1 mL/kg | 104 | μg/kg | 104 | mg/L | SC. | 10 |

Weekly total dose per treatment group:
5 μg/kg KBP-066 equals to 35 μg/kg/week or 10 nmol/kg/week
50 μg/kg KBP-066 equals to 350 μg/kg/week or 100.4 nmol/kg/week
500 μg/kg KBP-066 equals to 3500 μg/kg/week or 1004 nmol/kg/week
25 nmol/kg KBP-066 equals to 243.4 μg/kg/week or 58.3 nmol/kg/week
Compounds were dissolved in saline and stored at −20° C. Aliquots were thawed immediately prior to administration.

### Collection of Test Results

DAY −3: HbA1c measurement
DAY 1: (first day of study), rats were fasted for 6 h and a BG and blood sample was taken. Dosing was performed subsequently.
DAY 14: Fasting blood glucose (FBG)+blood sample (6 h fasting)
DAY 28: FBG+blood sample (6 h fasting)
DAY 42: FBG+blood sample (6 h fasting)
DAY 57: (gr. 1+2)/58 (gr. 3+4) OGTT with no pre-dosing of KBP-066 or KBP-066A11.03 (11 h fasting). Hb1Ac is measured during the OGTT at t=120 or t=180.
DAY 62: FBG+blood sample (6 h fast)

### Food Intake

Food intake was monitored daily. Body weight was monitored daily for first three weeks, then twice weekly after week three.

### Fasting Blood Glucose

Fasting blood glucose was monitored every two weeks using Accu-Check® Avia monitoring system (Roche Diagnostics, Rotkreuz, Switzerland): Measurement was taken from the tail vein (25 G needle).

### HbA1c

Rats were non-fasted for the first (randomization) and second (after the second OGTT) HbA1c measurement. A single drop of blood was applied to the HbA1c cassette and the HbA1c was measured using a DCA Vantage Analyzer. Dosing of compound or saline was performed subsequently during first and second HbA1c measurement.

### Oral Glucose Tolerance Test

A glucose tolerance test (OGTT) was performed after eight weeks of treatment. Body weight from the day prior was used to calculate glucose dose given. Animals were fasted for 11 h. Heat was applied app. 45 min prior to time point −30 min (see below figure). Animals were pre-dosed with KBP-066, KBP-066A11.03 or saline during the first OGTT but not in the second OGTT, hence (C) in the below figure.

### Results

### FIG. 6A+B, Accumulated Food Intake

FIG. 6A shows the accumulated food intake during the course of the study. All treatment groups eat less than the vehicle. Furthermore, higher doses leads to higher reduction in food intake. The acylated KBP-066A11.03 treatment group had the greatest reduction of food intake compared to KBP-066 at all dosages. At study end all treatment groups had a significant reduction in food consumed over the course of the study compared to vehicle, with acylated KBP-066A11.03 treatment showing the greatest reduction in food intake; ˜35% reduction in food intake vis-à-vis vehicle (FIG. 6B).

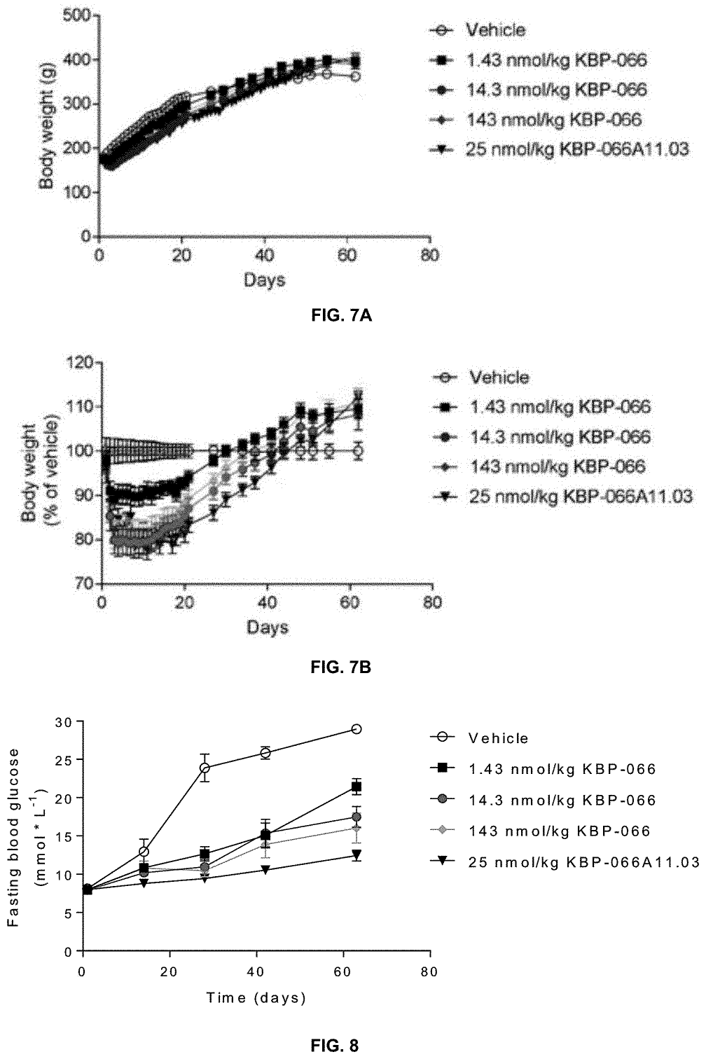

### FIG. 7A+B, Body Weight

All treatment groups lost body weight over the first three weeks of the study. As the ZDF vehicle rats became progressively sicker and thus failed to maintain their body weight/rate of gain (FIG. 7A) the treatment groups caught up to the vehicle group in terms of weight. The rate of weight gain compared to the vehicle was dose dependent as well as being dependent upon the type of compound used in the treatment group (FIG. 7B). The acylated KBP-066A11.03 treatment group has the slowest regain rate, followed by the 14.3 and 143 nmol/kg groups and with 1.43 nmol/kg as the fastest regainer of weight.

This shows that acylated KBP-66A11.03 given in a s.c dose regiment once every three days has additional pharmacological benefits over non-acylated KBP-066 given s.c., once daily.

### FIG. 8, Fasting Blood Glucose

As the ZDF vehicle rats became progressively sicker and failed to maintain FBG, all treatment groups attenuate FBG effectively for the duration of the study compared to vehicle. The acylated KBP-066A11.03 treatment was the most effective treatment, only allowing a modest 5 mM increase in FBG during the 62-day study in this super aggressive animal model of type 2 diabetes. The non-acylated KBP-066 reduced FBG in a dose dependent manner, but was not as potent as the acylated treatment group in attenuating FBG. Again, this shows that acylated KBP-66A11.03 has additional pharmacological benefits over non-acylated KBP-066.

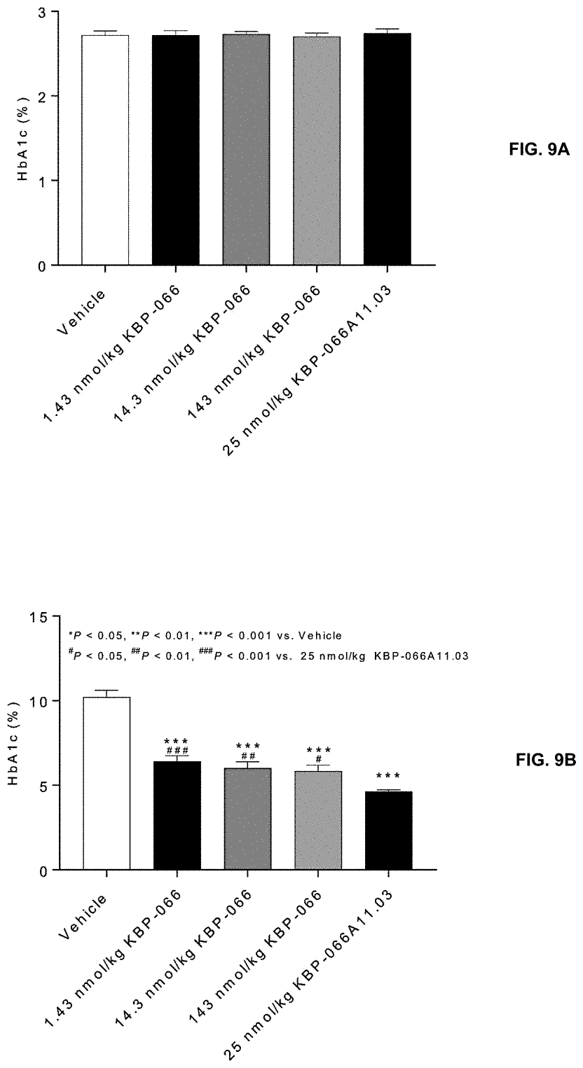

### FIG. 9, HbA1c at Baseline and Study End

As expected, HbA1c values at baseline are almost identical prior to onset of diabetes and treatment modalities in male ZDF rats (FIG. 9A). At study end (Day 62), all treatment groups had significantly reduced HbA1c levels compared to vehicle. Interestingly, the acylated KBP-066A11.03 treatment group had the lowest HbA1c values. Furthermore, it was also significantly lower than all the non-acylated KBP-066 treatment groups (FIG. 9B), demonstrating a further advantage of the acylation vis-à-vis the non-acylated equivalent.

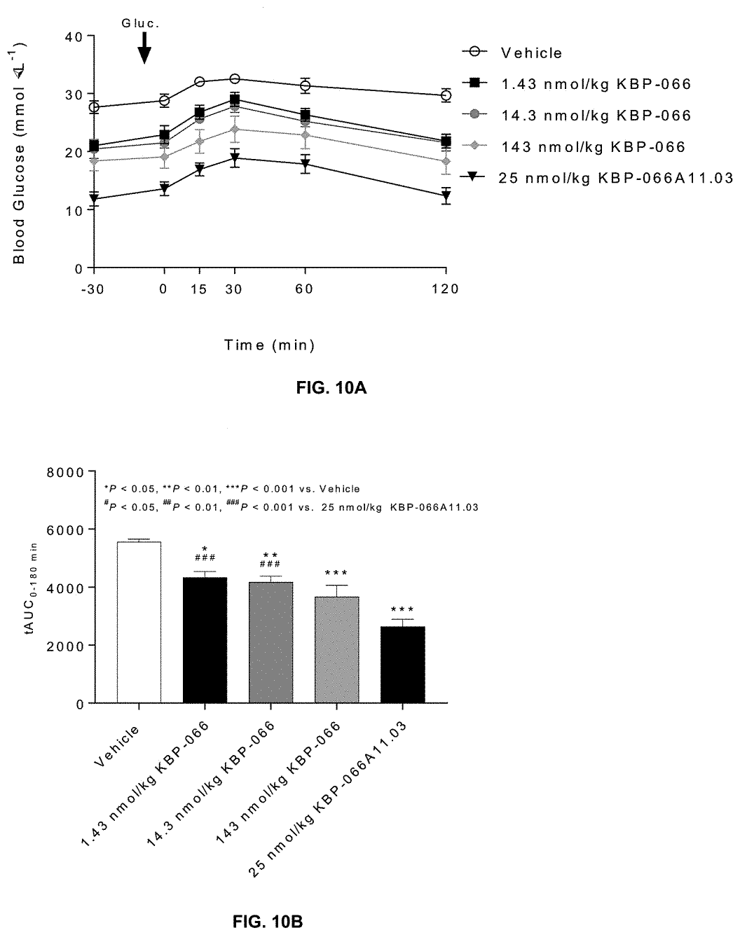

### FIG. 10, Oral Glucose Tolerance Test (OGTT)

An oral glucose tolerance test was conducted after eight weeks of treatment and results are illustrated in FIG. 10A. Due to treatment-induced differences in FBG the individual OGTT curves are markedly different. This difference is highlighted in the calculated tAUC values (FIG. 10B). All treatment groups have significantly lower tAUC compared to vehicle. The acylated KBP-066A11.03 treatment group had the lowest tAUC value, and was also significantly lower than two of three the non-acylated KBP-066 treatment groups, and with a p-value of 0.06 when compared to the last group, 143 nmol/kg KBP-066 (FIG. 10B).

In conclusion, the collective data show that acylated KBP-66A11.03 given in a s.c dose regiment every three days have significantly advantageous additional pharmacological benefits over non-acylated KBP-066 given s.c. once daily in obese and diabetic ZDF rats.

### Summary of Results of Examples 1-6

### Results of Acylation Studies by Acylation Site

### Position A09 (Acylation at the 9 Position of the Peptide)

Acylation of position A09 with 1 acylation produced a sustained prolonged in vivo activity that merited further testing (FIG. 1A-F).

Furthermore, acylation of position A09 with 2 and 3 acylations attenuated EC50 on both the CTR and AMYR receptor and produced no prolonged response on the CTR (Table 3-4).

However, acylation of A09 with 2 and 3 acylations disrupted the previously observed prolonged in vivo efficacy of the core peptide making the potency of the acylated KBP similar to that of vehicle. Hence, they were less potent than the non-acylated core peptide (FIG. 2A-B) in both reducing both food intake and body weight after a single s.c. injection of 36 nmol/kg compound.

### Position A09 was Therefore not Given any Further Consideration.

Position A11 (acylation at the 11 position of the peptide) Acylation of position A11 with the 1 acylation produced a sustained prolonged in vivo activity that merited further testing (FIG. 1A-F).

Acylation of position A11 with acylations 2 and 3 resulted in the best assayed EC50 value on both the CTR and AMYR receptor, and producing the highest prolonged response values (tAUC) across all core peptides tested (Table 3-4).

Furthermore, A11/3 acylations improved the in vivo activity of the core peptide significantly compared to the non-acylated core peptide in both reducing food intake (FIG. 2A) and body weight (FIG. 2B) after a single s.c. injection of 36 nmol/kg compound. A11/3 acylations were also better at reducing food intake and body weight than any other acylated positions when using KBP-089 as core peptide (FIG. 2A-B).

This difference was further underscored in a dose response test (FIG. 3A-F) in which three doses (4 nmol/kg, 12 nmol/kg and 36 nmol/kg) were all superior to the corresponding A32/3 acylated peptides. In terms of potency, the lowest dose of 4 nmol/kg A11/3 had a similar profile to the 36 nmol/kg A32/3 acylated peptides. This was consistently demonstrated for all tested core peptides (FIG. 3A-F).

To further investigate the potency of the A11 position with the 3 acylation, KBP-042 and KBP-066 acylated at position A11 with the 3 acylation was tested at a high dose (300 nmol/kg) and compared to the non-acylated versions to demonstrate the potential maximum effect of the protracted in vivo efficacy combined with the protracted bio-availability (FIG. 4A-D).

Acylation 3 at position A11 attenuated food intake for more than 120 hours returning to vehicle food consumption levels after \hat{ }144 hours for both KBP-042 (FIG. 4A) and KBP-066 (FIG. 4C). Treatment mediated body weight loss peaked after 96 hours and body weight returned to baseline levels after \hat{ }240 hours for both KBP-042 (FIG. 4B) and KBP-066 (FIG. 4D).

In conclusion, A11 was the best position tested in terms of preserving ligand potency and maximizing the protracted in vivo efficacy.

### Position A12 (Acylation at the 12 Position of the Peptide)

A12 position with a 1 acylation produced a worse result in vivo in the 4 h food intake study when compared to the vehicle (FIG. 5).

Thus, position A12 was not a good candidate for acylation, and was not tested further.

### Position A16 (Acylation at the 16 Position of the Peptide)

A16 position with a 1 acylation demonstrated no prolonged activity in vivo (FIG. 1A-F).

Thus, position A16 was not a good candidate using 1 acylation, and was not tested further.

### Position A18 (Acylation at the 18 Position of the Peptide)

A18 position with a 1 acylation was efficacious across the 4-24 hour testing period, however the observed efficacy was not maintained in the prolonged activity study (KBP-347, 48 h, FIG. 1F).

Thus, position A18 was not a good candidate using 1 acylation, and was not tested further.

### Position A32 (Acylation at the 32 Position of the Peptide)

A32 position with a 1 acylation demonstrated a prolonged effect in vivo on both food intake and body weight, and was among the best of the tested compounds (FIG. 1A-F).

Position A32 with acylation 2 and 3 resulted in inferior assayed EC50 values on both the CTR and AMYR receptor compared to position A11. Acylation of position A32 attenuated the CTR mediated prolonged response slightly compared to position A11, but still maintained a prolonged response (Table 3-4).

Acylations at position A32 improved the in vivo efficacy of a single dose s.c. treatment compared to the non-acylation counterparts for all tested core peptides.

However, the position was inferior to A11 in all tested 2 and 3 acylations during in vivo studies at equivalent doses (FIG. 2A-B, FIG. 3A-F).

In conclusion, A32 was a mediocre position in terms of preserving ligand potency and improving in vivo efficacy using 1, 2 and 3 acylations when compared to position A11.

### Further Acylation Studies (Examples 7-12)

### Example 7: μ-Arrestin and Thioflavin T Assays

Additional PathHunter β-Arrestin GPCR assays were carried out, using the same protocol as described above in connection with Example 2. In independent bioassays, CTR and AMY-R cells were treated at the indicated time points with increasing doses of KBPs identified in Tables 4.1-4.4 (ranging from 1 μM-0.1 nM and vehicle).

Thioflavin T assays were also conducted. Thioflavin T (ThT) is a dye widely used for the detection of amyloid fibrils. In the presence of fibrils, ThT has an excitation maximum at 450 nm and enhanced emission at 480 nm, whereas ThT is essentially non-fluorescent at these wavelengths when not bound to amyloid fibrils.

Thus, ThT in combination with a fluorescent plate reader is an ideal tool for screening large numbers of in vitro samples for the presence of amyloid fibrils. The ThT assay used for the KBPS was a modification of the procedure described by Nielsen et. al. (Nielsen L, Khurana R, Coats A, FrØkjaer S, Brange J, Vyas S, et al. Effect of environmental factors on the kinetics of insulin fibril formation: elucidation of the molecular mechanism. Biochemistry. 2001; 40 (20): 6036-46.1) for measuring insulin fibrillation.

Fibrillation screening assays were conducted in 384-well plates (Greiner Bio-One, 784080) in sample triplicates with a final volume of 20 μL. The plate is sealed using an optical adhesive film to prevent sample evaporation over the course of the assay.

The plate is loaded into a fluorescent plate reader, such as a SpectraMax with SoftMax Pro 7.0.2 software, and the template set to 37° C. with excitation wavelength at 450 nm and emission wavelength at 480 nm.

Plate reader should measure fluorescence every 10 minutes for 24 hours with a five-second plate shake before the first read and a three-second plate shake before all other reads. Alternatively, the plate is read after the following incubation times; 0, 1, 2, 4 and 24 hours.

Plot relative fluorescence units (RFU) as a function of time. Fibrillation is determined as an increase in RFU over baseline as described by Nielsen et. al.

In this filing four fibrillations tiers have been defined based on the 18 h fluorescence signal to get a single output that reflects the peptides fibrillation potential: None=<1000 RFU, Low=1000-3000 RFU, Medium=3000-10000, High=>10000

The results of the Thioflavin T assays are also shown in Tables 4.1-4.4.

TABLE 4.1
β-arrestin study for different acylations length (KAc-(glutamic acid linker)-(C14 to C26 diacid))

| | | | Compounds | | | | | | | |
|---|---|---|---|---|---|---|---|---|---|---|
| | | | U2OS | | CHO-K1 | | Peptide | | Food | |
| | | | (CTR) | | (AMY-R) | | Fibrillation | | Intake | |
| | | | Acylated KBPs | | | | | | | |
| | | | β-arrestin | | β-arrestin | | Thioflavin T | | ΔFOOD | |
| | | | Fold | | Fold | | ΔFluorescence | | Sustained | |
| | | | Recruitment | | Recruitment | | 18 h | | Attenuation | |
| | Core | Acylation | EC50 values | | EC50 values | | Assay | | (4 nmol/kg) | |
| NO | Sequence | Type | (10⁻⁹ M) | | (10⁻⁹ M) | | Score | | Hours (h) | |
| 356 | KBP-066 | A11.03 | 3.0 ± 2.4 | (23) | 7.2 ± 4.7 | (26) | None | (10) | 72-96 | h |
| 383 | KBP-066 | A11.04 | 33.0 ± 10.3 | (3) | 16.6 ± 2.9 | (3) | None | (3) | 96 | h |
| 382 | KBP-066 | A11.05 | 56.1 ± 10.6 | (3) | 23.3 ± 4.2 | (3) | None | (3) | 96 | h |
| 381 | KBP-066 | A11.06 | 3.9 ± 0.9 | (3) | 1.0 ± 0.5 | (3) | None | (3) | 24-48 | h |
| 307 | KBP-066 | A11.09 | 26.6 ± 6.1 | (3) | 149 ± 127 | (4) | None | (3) | 72 | h |
| 306 | KBP-066 | A11.10 | 65.7 ± 2.9 | (3) | 82.7 ± 6.6 | (4) | None | (3) | 48 | h |
| 305 | KBP-066 | A11.11 | 2.1 ± 1.0 | (3) | 0.95 ± 0.2 | (4) | None | (3) | 24 | h |

Table 4.1: In vitro peptide screening characteristics

TABLE 4.2
β-arrestin study for different acylations positions using backbone
(KBP-066) and 3 acylation (KAc-(glutamic acid linker)-(C18 diacid))

| | | | Compounds | | | | | | | |
|---|---|---|---|---|---|---|---|---|---|---|
| | | | U2OS | | CHO-K1 | | Peptide | | Food | |
| | | | (CTR) | | (AMY-R) | | Fibrillation | | Intake | |
| | | | Acylated KBPs | | | | | | | |
| | | | β-arrestin | | β-arrestin | | Thioflavin T | | ΔFOOD | |
| | | | Fold | | Fold | | ΔFluorescence | | Sustained | |
| | | | Recruitment | | Recruitment | | 18 h | | Attenuation | |
| | Core | Acylation | EC50 values | | EC50 values | | Assay | | (4 nmol/kg) | |
| NO | Sequence | Type | (10⁻⁹ M) | | (10⁻⁹ M) | | Score | | Hours (h) | |
| 354 | KBP-066 | A09.03 | 4.7 ± 0.6 | (3) * | 93.0 ± 26 | (3) * | None | (3) | 4 | h |
| 356 | KBP-066 | A11.03 | 3.0 ± 2.4 | (23) | 7.2 ± 4.7 | (26) | None | (10) | 72-96 | h |
| 386 | KBP-066 | A12.03 | 56.5 ± 21.1 | (3) | 98.9 ± 74.8 | (3) | None | (3) | 0-4 | h |
| 387 | KBP-066 | A16.03 | 25.4 ± 9.9 | (3) | 21.1 ± 9.9 | (3) | Low | (3) | 48 | h |
| 388 | KBP-066 | A18.03 | 22.8 ± 11.6 | (3) | 34.7 ± 33.8 | (3) | None | (3) | 72 | h |
| 389 | KBP-066 | A19.03 | 9.4 ± 3.2 | (3) | 6.5 ± 3.1 | (3) | None | (3) | 72-96 | h |
| 390 | KBP-066 | A24.03 | 11.1 ± 2.8 | (3) | 6.0 ± 3.3 | (3) | None | (3) | 72 | h |
| 358 | KBP-066 | A32.03 | 44.2 ± 5.7 | (3) * | 98.6 ± 53 | (3) * | None | (3) | 72 | h |

Table 4.2: In vitro peptide screening characteristics
* Data from original patent filing

TABLE 4.3
β-arrestin study for different acylations positions using backbone
(KBP-021) and 3 acylation (KAc-(glutamic acid linker)-(C18 diacid))

| | | | Compounds | | | | | | | |
|---|---|---|---|---|---|---|---|---|---|---|
| | | | U2OS | | CHO-K1 | | Peptide | | Food | |
| | | | (CTR) | | (AMY-R) | | Fibrillation | | Intake | |
| | | | Acylated KBPs | | | | | | | |
| | | | β-arrestin | | β-arrestin | | Thioflavin T | | ΔFOOD | |
| | | | Fold | | Fold | | ΔFluorescence | | Sustained | |
| | | | Recruitment | | Recruitment | | 18 h | | Attenuation | |
| | Core | Acylation | EC50 values | | EC50 values | | Assay | | (4 nmol/kg) | |
| NO | Sequence | Type | (10⁻⁹ M) | | (10⁻⁹ M) | | Score | | Hours (h) | |
| 312 | KBP-021 | A09.03 | 16.7 ± 2.9 | (3) | 1528 ± 1201 | (3) | Low | (3) | 4 | h |
| 391 | KBP-021 | A11.03 | 12.5 ± 10.9 | (9) | 9.0 ± 2.2 | (5) | None | (3) | 72-96 | h |
| 393 | KBP-021 | A11.04 | 55.1 ± 48.9 | (3) | 32.8 ± 14.6 | (4) | Low | (3) | 72-96 | h |
| 394 | KBP-021 | A11.05 | 56.9 ± 33.4 | (3) | 53.9 ± 21.2 | (4) | Low | (3) | 24 | h |
| 313 | KBP-021 | A12.03 | 314 ± 116 | (3) | 330 ± 124 | (4) | None | (3) | 4 | h |
| 314 | KBP-021 | A16.03 | 183 ± 149 | (3) | 428 ± 175 | (5) | None | (3) | 4 | h |
| 315 | KBP-021 | A18.03 | 19.2 ± 6.6 | (3) | 44.9 ± 6.6 | (5) | Medium | (3) | 48 | h |
| 316 | KBP-021 | A19.03 | 2.5 ± 1.4 | (3) | 6.1 ± 1.8 | (5) | High | (3) | 72-96 | h |
| 395 | KBP-021 | A19.05 | 95.2 ± 95.2 | (3) | 32.9 ± 12.7 | (4) | High | (3) | 72-96 | h |
| 317 | KBP-021 | A24.03 | 12.8 ± 12.8 | (3) | 31.5 ± 7.4 | (5) | None | (3) | 24 | h |
| 318 | KBP-021 | A32.03 | 197 ± 83.6 | (3) | 301 ± 225 | (5) | Low | (3) | 4 | h |

Table 4.3: In vitro peptide screening characteristics

TABLE 4.4
β-arrestin study for different acylations linkers using
same backbone (KBP-066) and same acylation(C18 diacid))

| | | | Compounds | | | | | | | |
|---|---|---|---|---|---|---|---|---|---|---|
| | | | U2OS | | CHO-K1 | | Peptide | | Food | |
| | | | (CTR) | | (AMY-R) | | Fibrillation | | Intake | |
| | | | Acylated KBPs | | | | | | | |
| | | | β-arrestin | | β-arrestin | | Thioflavin T | | ΔFOOD | |
| | | | Fold | | Fold | | ΔFluorescence | | Sustained | |
| | | | Recruitment | | Recruitment | | 18 h | | Attenuation | |
| | Core | Acylation | EC50 values | | EC50 values | | Assay | | (4 nmol/kg) | |
| NO | Sequence | Type | (10⁻⁹ M) | | (10⁻⁹ M) | | Score | | Hours (h) | |
| 385 | KBP-066 | A11.07 | 20.1 ± 11.1 | (3) | 9.3 ± 1.7 | (2) | None | (3) | 72 | h |
| 384 | KBP-066 | A11.08 | 29.7 ± 29.2 | (3) | 6.9 ± 1.1 | (3) | Low | (3) | 72 | h |
| 356 | KBP-066 | A11.03 | 3.0 ± 2.4 | (23) | 7.2 ± 4.7 | (26) | None | (10) | 72-96 | h |

Table 4.4: In vitro peptide screening characteristics

### Results—Acylation Length

In terms of in vitro potency as a function of acylation length there was a clear correlation between acylation length and in vitro potency. EC50 values on both the CTR and AMYR by the shortest acylations, 11(C14 diacid) and 6 (C16 diacid), produced the lowest EC50s on both receptors (Table 4.1), whereas the longest acylations, 9 (C24 diacid) and 10 (C26 diacid), produced some of the highest recorded EC50 values on both receptors.

None of the tested acylated peptides in this series using the KBP-066 backbone had any fibrillation issues.

### Results—Acylation Position on the KBP-066 Backbone

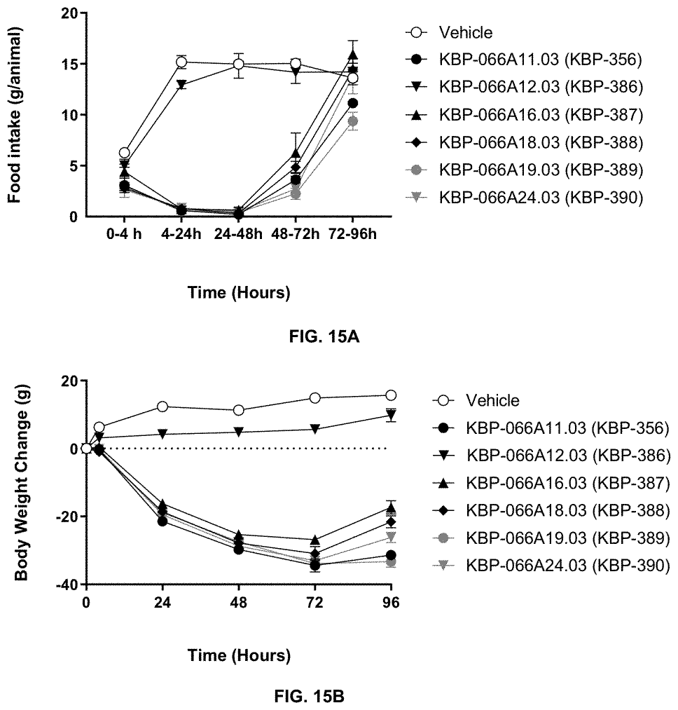

EC50 values on the CTR and AMYR on this series are listed in Table 4.2. In terms of in vitro potency as a function of acylation position on the KBP-066 backbone, three positions stand out as potent dual calcitonin and amylin receptor agonists. All, A19 and A24 all have EC50 values on both receptors in the 5×10⁻⁹ M range as the only ones, whereas all other tested positions are impaired in comparison. The increased potency of A11, A19 and A24 appears to translate into improved in vivo efficacy for the KBP-066 backbone (see FIGS. 15, 17 and 18 described infra). Interestingly, A09 is among the best CTR agonist, but has poor AMYR activity, suggesting that an acylation to close to the N-terminus can disrupt AMYR activity and generate a biased ligand.

Fibrillation does not appear to be an issue for the KBP-066 backbone at most positions, as only one peptide (KBP-066A16.03 (387)) produced a “Low” score in the ThT assay.

### Results—Acylation Position on the KBP-021 Backbone

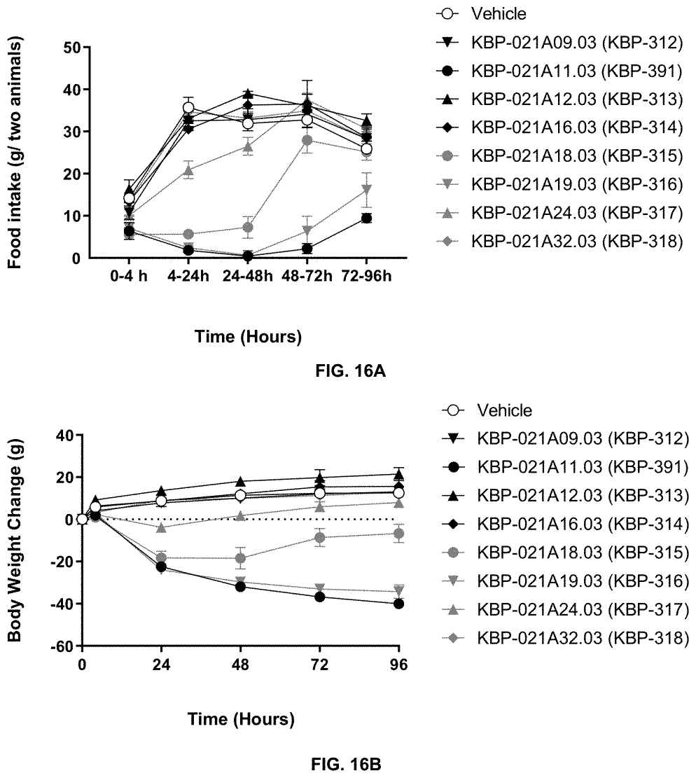

EC50 values on the CTR and AMYR for this series are listed in Table 4.3. In terms of in vitro potency as a function of acylation position on the KBP-021 backbone, two positions stand out as potent dual agonists. All and A19 both have EC50 values on both receptors in the 5×10⁻⁹ M range as the only ones, whereas all other tested positions are impaired in comparison. The increased potency of A11 and A19 also appear to translate into improved in vivo efficacy for the KBP-021 backbone (see FIG. 16 described infra).

Interestingly, fibrillation appears to be an issue for the KBP-021 backbone, where position A19 as the only peptide tested scored a “High” score in the ThT assay despite good potency both in vitro and in vivo. The position next to it “A18” also scored high with a “Medium” score in the ThT assay suggesting the KBP-021 backbone is susceptible to fibrillation when acylated in that area of the backbone.

Furthermore, longer acylations also appear to increase fibrillation for this backbone, KBP-021, as the 4 and 5 acylation on position A11, scored a “Low” score in the ThT assay, however, this issue did not affect the favoured position A11 with 3 acylation.

### Results—Acylation Linker

EC50 values on the CTR and AMYR for this series are listed in Table 4.4. In terms of in vitro potency as a function of acylation position on the KBP-066 backbone, the OEG-OEG-γGLU linker (356) have an almost 10-fold better EC50 on the CTR compared to OEG-OEG-OEG-γGLU (385) and OEG-γGLU (384), however, all linkers have very similar EC50 in the 5×10⁻⁹ M range on the AMYR.

In terms of fibrillation, the shortest linker, OEG-γGLU (384), produced a “low” score in the ThT assay, whereas the two other linkers produce a “None” score.

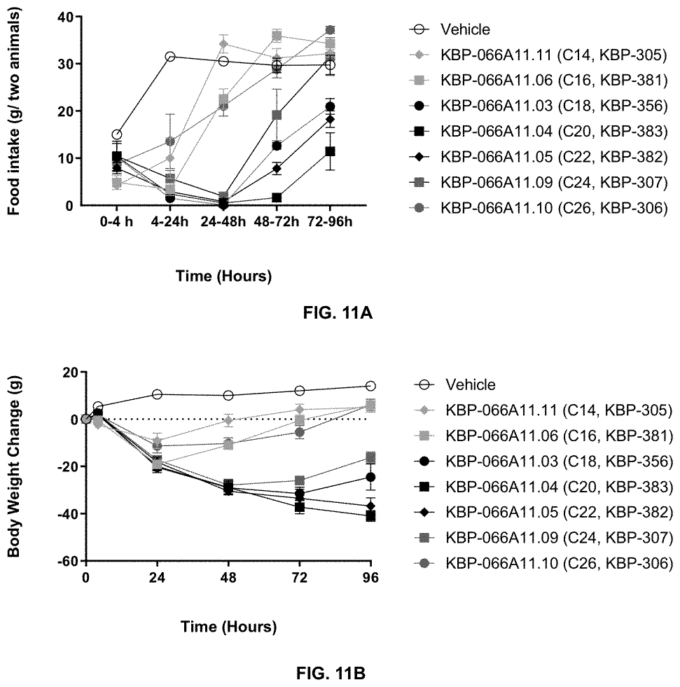

### Example 8: (FIG. 11)

Single dose comparative effect of several acylated variants (3, 4, 5, 6, 9, 10, 11) at the same position and backbone, A11 and KBP-066, respectively, on food intake and body weight in an acute setting in 20-week old SD rats feed HFD for 8 weeks prior to the experiment

| | | Acylation | | Position/ |
|---|---|---|---|---|
| KBP | Core | length | Annotation | Acylation |
| KBP-356 | KBP-066 | C18 diacid | KBP-066A11.03 | A11/3 acylation |
| KBP-383 | KBP-066 | C20 diacid | KBP-066A11.04 | A11/4 acylation |
| KBP-382 | KBP-066 | C22 diacid | KBP-066A11.05 | A11/5 acylation |
| KBP-381 | KBP-066 | C16 diacid | KBP-066A11.06 | A11/6 acylation |
| KBP-307 | KBP-066 | C24 diacid | KBP-066A11.09 | A11/9 acylation |
| KBP-306 | KBP-066 | C26 diacid | KBP-066A11.10 | A11/10 acylation |
| KBP-305 | KBP-066 | C14 diacid | KBP-066A11.11 | A11/11 acylation |

Rats were single caged four days prior to the test. Rats were randomized by weight into eight groups (Vehicle (0.9% NaCl), KBPs (doses: 3 nmol/kg (\hat{ }10-11 μg/kg)). They were fasted overnight and then treated with a single dose of peptide or vehicle in the morning using subcutaneous administration. Food intake was monitored in the following intervals (0-4 hours, 4-24 hours, 24-48 hours, 48-72 hours, and 72-96 hours). Body weight was measured at baseline and at 4 hour, 24 hours, 48 hours, 72 hours and 96 hours post s.c injection.

### FIG. 11 Results—Food Intake and Body Weight

Acylation 6, 10, 11 are able attenuate food intake and body weight with a peak suppression at 24 hours followed by rebound to vehicle levels. Acylation 9 is able attenuate food intake and body weight with a peak suppression at 48 hours followed by rebound to vehicle levels. Acylation 3 is able attenuate food intake and body weight with a peak suppression at 72 hours followed by rebound to vehicle levels. Acylation 4 and 5 were able to attenuate food intake and body weight with a peak suppression after 96 hours followed by a rebound.

Hence, acylation 3, 4, and 5 are all prime candidates for acylation length as the initial goal was to suppress food intake and body weight for a minimum of 72 hours as every 3^{rd} day dosing in rodents appears to translate into once weekly dosing in man.

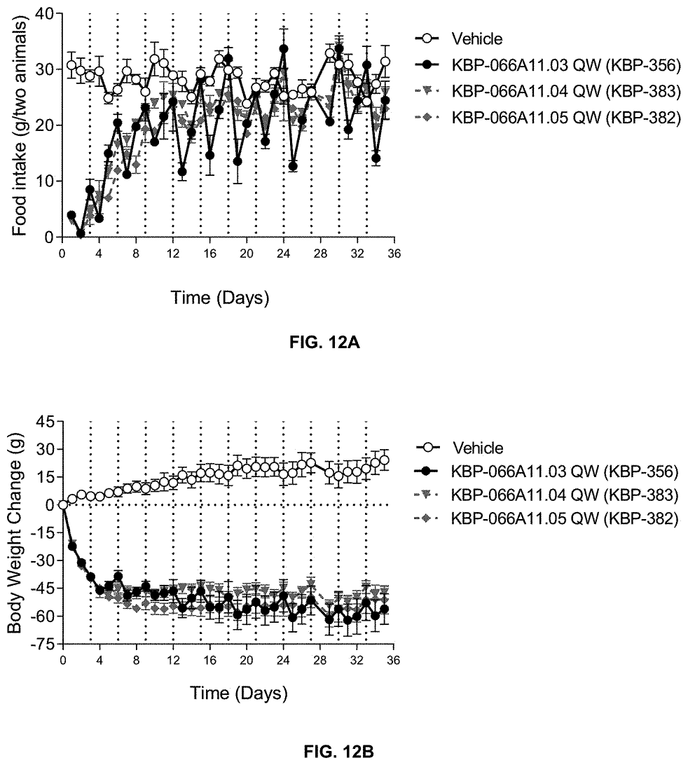

### Example 9 (FIGS. 12, 13 and 14)

Further work was conducted on the best performers from the acute testing, acylated variants (3, 4, 5), and a study using repeated dosing for comparative effect of the acylations with the same position and backbone, A11 and KBP-066 respectively, was carried out. Food intake and body weight were investigated in a chronic setting (five-week study) in 20-week old SD rats feed HFD for 8 weeks prior to study start.

| | | Acylation | | Position/ |
|---|---|---|---|---|
| KBP | Core | length | Annotation | Acylation |
| KBP-356 | KBP-066 | C18 diacid | KBP-066A11.03 | A11/3 acylation |
| KBP-383 | KBP-066 | C20 diacid | KBP-066A11.04 | A11/4 acylation |
| KBP-382 | KBP-066 | C22 diacid | KBP-066A11.05 | A11/5 acylation |

Rats were caged two and two and were randomized by weight into treatment groups (Vehicle (0.9% NaCl), KBPs (doses: 3 nmol/kg (\hat{ }14 μg/kg)). Food intake and body weight were monitored daily for 35 days. At study end, an OGTT was performed followed by animal termination, in which, adipose tissue was taken out and weighed.

### Chronic Treatment of Male HFD SD Rats

Rats were delivered to the animal facility of Nordic Bioscience at twelve weeks of age and immediately put on HFD and fed on it for an additional eight weeks. Prior to study start the rats were randomized based on body weight. The study was initiated at DAY 1.

### Dosage Concentrations and Frequency

Animals were dosed with KBPs once every third day. Dosing was administered subcutaneously (SC) around noon every day. Compounds were dissolved in saline and stored at −20° C. Aliquots were thawed immediately prior to administration.

Saline: Dosage volume was 1 mL/kg.
KBPs: Dosage volume was 1 mL/kg, Dosage concentration was 4 nmol/kg.

The dose equivalent in μg/kg was \hat{ }14 μg/kg. Weekly total dose per treatment group: 4 nmol/kg KBP equals to 28 nmol/kg/week or \hat{ }100 μg/kg/week

### Collection of Test Results

DAY 1: (first day of study dosing was performed
Day 1-35: Daily monitoring of food intake and body weight
DAY 35: Body weight at study end
DAY 35: Oral Glucose tolerance test
DAY 35: Termination+adipose tissue weighed

### Oral Glucose Tolerance Test

A glucose tolerance test (OGTT) was performed after five weeks of treatment. Body weight from the day prior was used to calculate glucose dose given. Animals were fasted for 11 h. Heat was applied app. 45 min prior to time point −30 min (see below figure). Animals were dosed with KBPs or vehicle the day before the OGTT.

### White Adipose Tissue (WAT) Weighing

The entire epididymal and perirenal WAT depot was dissected out and weighed. For Inguinal WAT, a fixed anatomical limited area was dissected out and weighed.

### FIG. 12 Results, Food Intake and Body Weight

FIG. 12 shows the change in food intake and body weight over time during the chronic study as a function of treatment. FIG. 12A shows the dynamic in food intake between acylation 3, 4 and 5, whereas FIG. 12B shows the body weight loss mediated by acylation 3, 4 and 5. It is evident that all three acylations give a significant reduction in body weight after 5 weeks of treatment, however, there are no differences between acylation 3, 4 and 5 in terms on efficacy on body weight.

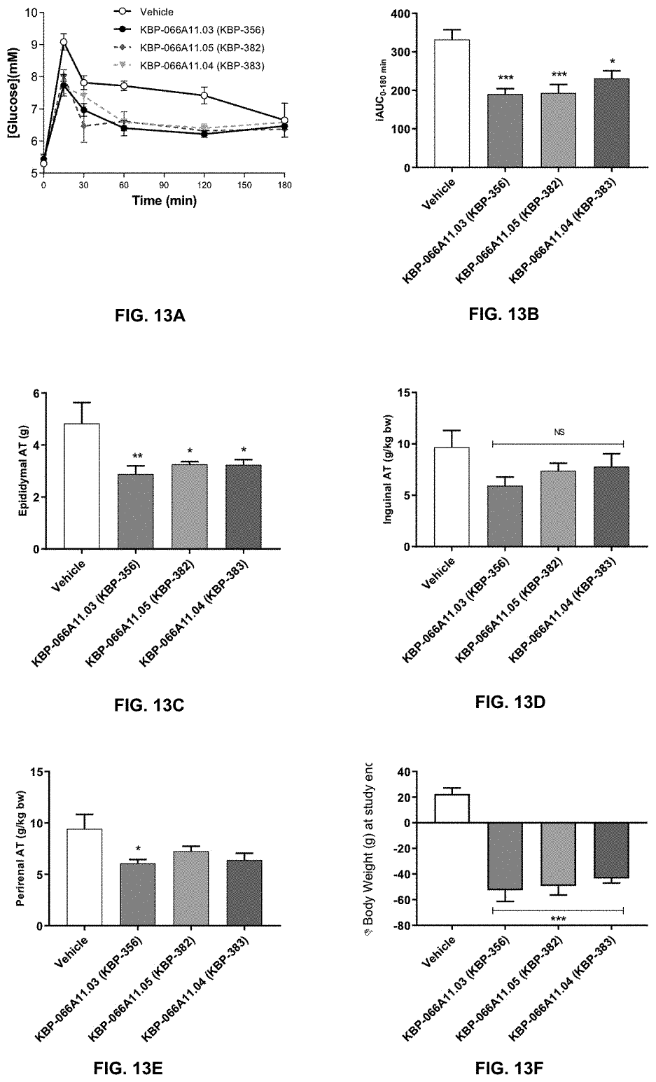

### FIG. 13 Results, OGTT and Adipose Tissue

FIG. 13 shows the results from the OGTT with the corresponding iAUC (OGTT)(Figure A+B) was well as the weight of three different adipose tissue, epididymal, inguinal and perirenal (FIG. 13C-E) and the body weight as study end (FIG. 13F). Treatment with acylation 3 resulted in a significant reduction in iAUC (OGTT), epididymal WAT size, perinal WAT size, and body weight as study. Treatment with acylation 4 and 5 resulted in a significant reduction in iAUC (OGTT), epididymal WAT size, and body weight at study end, but not in perirenal WAT size. Neither treatment significantly reduced the size of the inguinal WAT. Acylation 3 performed slightly better against vehicle compared to 4/5, but there were no significant differences between treatment groups.

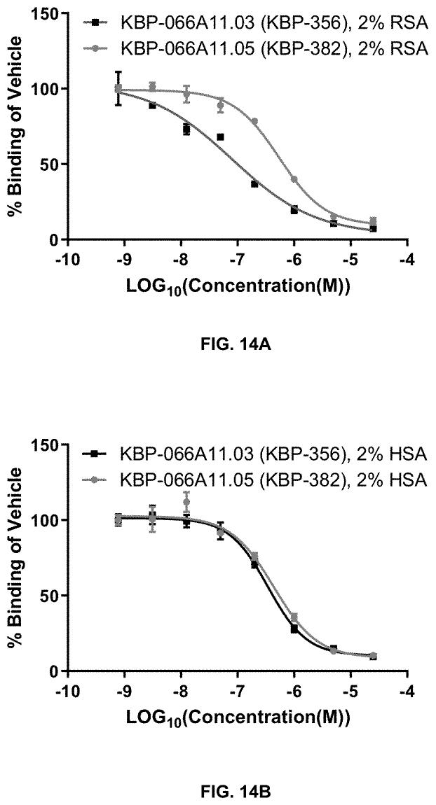

### FIG. 14 Results, Competitive 1-125 sCT Ligand Binding

To investigate whether the improved efficacy in the acute setting of acylation 4 and 5 could be translated to man, a competitive ligand binding assay was conducted to explore acylation binding to serum albumin in rodent and man. FIG. 14 shows the competitive binding of KBP-066A11.03 and KBP-066A11.05 with radio-labelled I-125 salmon calcitonin (NEX423, Perkin Elmer) in 2% serum albumin from rodents (RSA) (FIG. 4A) or 2% serum albumin from humans (HSA)(FIG. 4B). When the assay is conducted 2% HSA there are no differences in EC₅₀ between acylations 3 and 5. However, when the assay was conducted in RSA, the 5 acylation shifted the IC50 further to the right suggesting a stronger affinity towards the RSA in the assay. Hence, the improved efficacy observed in the acute setting in rodents is most likely a phenomenon unique to rodents and not translational to man.

### Example 10 (FIG. 15)

Single dose comparative effect of 3 acylated variants at different positions (9 position “A09”, 11 position “A11”, 12 position “A12”, 16 position “A16”, 18 position “A18”, 19 position “A19”, and 32 position “A32”) to one another on food intake and body weight in 20 week HFD SD rats.

| | | | Position/ |
|---|---|---|---|
| KBP | Core | Annotation | Acylation |
| KBP-354 | KBP-066 | KBP-066A09.03 | A9/3 acylation |
| KBP-356 | KBP-066 | KBP-066A11.03 | A11/3 acylation |
| KBP-386 | KBP-066 | KBP-066A12.03 | A12/3 acylation |
| KBP-387 | KBP-066 | KBP-066A16.03 | A16/3 acylation |
| KBP-388 | KBP-066 | KBP-066A16.03 | A18/3 acylation |
| KBP-389 | KBP-066 | KBP-066A18.03 | A19/3 acylation |
| KBP-390 | KBP-066 | KBP-066A19.03 | A24/3 acylation |
| KBP-358 | KBP-066 | KBP-066A24.03 | A32/3 acylation |

Rats were single caged four days prior to the test. Rats were randomized by weight into eight groups (Vehicle (0.9% NaCl), KBPs (doses: 4 nmol/kg (\hat{ }10-11 μg/kg)). They were fasted overnight and then treated with a single dose of peptide or vehicle in the morning using subcutaneous administration. Food intake was monitored in the following intervals (0-4 hours, 4-24 hours, 24-48 hours, 48-72 hours, and 72-96 hours). Body weight was measured at baseline and at 4 hour, 24 hours, 48 hours, 72 hours and 96 hours post s.c. injection. Two backbones were tested, KBP-066 and KBP-021.

### FIG. 15 Results—Food Intake and Body Weight

In terms of position on backbone, the KBP-066 results are as follows. At 4 nmol/kg in an acute setting (FIG. 5), position A11 and A19 were the two-best positions at suppressing both food intake for 72 hours and body weight for 96 hours. Third best position was A24, followed by A18 and A16. The least potent position to acylate was A12 which looks like a disadvantageous position to acylate as it appears to somewhat interfere with the DACRA mediated efficacy on both food intake and body weight.

Based on these data, A11 and A19 are the preferred positions to acylate backbone KBP-066.

### FIG. 16 Results—Food Intake and Body Weight

When using a different backbone, KBP-021, with the same experimental settings as for KBP-066, the position pattern was slightly different.

| | | | Position/ |
|---|---|---|---|
| KBP | Core | Annotation | Acylation |
| KBP-312 | KBP-021 | KBP-021A09.03 | A9/3 acylation |
| KBP-391 | KBP-021 | KBP-021A11.03 | A11/3 acylation |
| KBP-313 | KBP-021 | KBP-021A12.03 | A12/3 acylation |
| KBP-314 | KBP-021 | KBP-021A16.03 | A16/3 acylation |
| KBP-315 | KBP-021 | KBP-021A16.03 | A18/3 acylation |
| KBP-316 | KBP-021 | KBP-021A18.03 | A19/3 acylation |
| KBP-317 | KBP-021 | KBP-021A19.03 | A24/3 acylation |
| KBP-318 | KBP-021 | KBP-021A24.03 | A32/3 acylation |

At 3 nmol/kg in an acute setting (FIG. 6), position A11 and A19 were by far the two-best positions at suppressing both food intake for 72 hours and body weight for 96 hours like what was observed for the KBP-066 backbone. Third best position was A18, but it was far inferior when compared to A11 and A19. Position A24 was better than vehicle, but nowhere near what was observed for the KBP-066 backbone. Position A16, A12 and A09 all failed to separate from vehicle on both food intake and body weight suggesting the positions they were acylated were disadvantageous as they inferred with peptide activity.

However, as the in vitro characteristics table 4.3 shows, A19 and A18 have major issues in terms of fibrillation potential in combination with KBP-021, which make A11 the preferred position to acylate when it comes to backbone KBP-021.

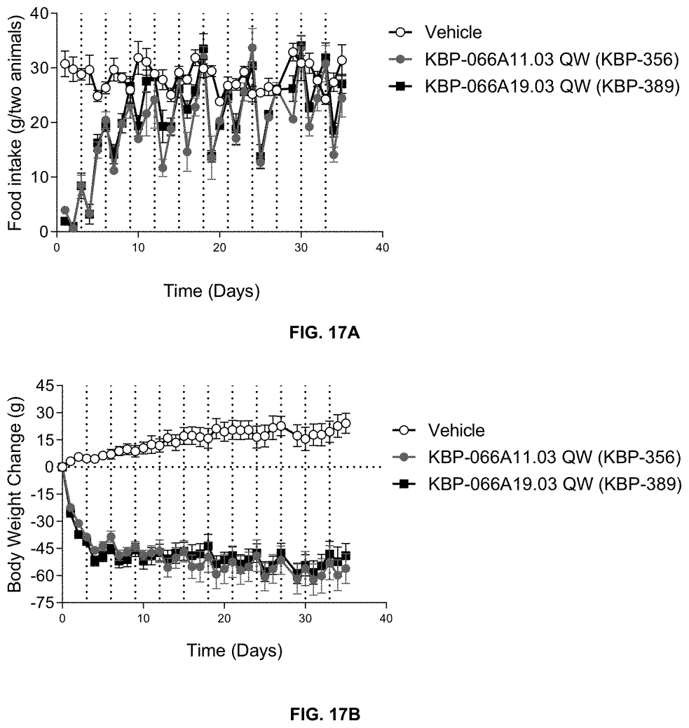

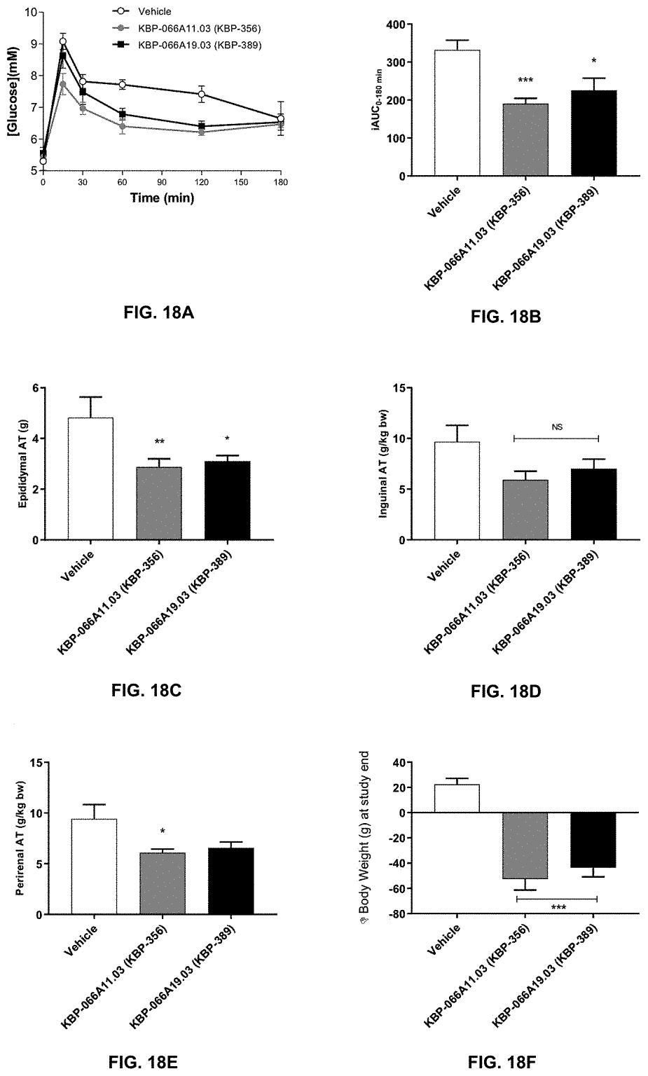

### Example 11 (FIGS. 17 and 18)

Further work was conducted on the best performers from the acute testing, acylated positions (A11 and A19), and a study using repeated doses for comparative effect of the acylations with the same acylation and backbone, namely 3 acylation and KBP-066, respectively.

Food intake and body weight were investigated in a chronic setting (five-week study) in 20-week old SD rats feed HFD for 8 weeks prior to study start.

| | | Acylation | | Position/ |
|---|---|---|---|---|
| KBP | Core | Position | Annotation | Acylation |
| KBP-356 | KBP-066 | A11 | KBP-066A11.03 | A11/3 acylation |
| KBP-389 | KBP-066 | A19 | KBP-066A19.03 | A19/3 acylation |

The experimental protocol as described above in Example 9 was followed. Briefly, rats were caged two and two and were randomized by weight into treatment groups (Vehicle (0.9% NaCl), KBPs (doses: 4 nmol/kg (\hat{ }14 μg/kg)). Food intake and body weight were monitored daily for 35 days. At study end, an OGTT was performed followed by animal termination in which adipose tissue was taken out and weighed.

### FIG. 17 Results—Food Intake and Body Weight for all Vs A19 in a Chronic Setting

FIG. 17 shows the change in food intake (FIG. 17A) and in body weight (FIG. 17B) over time during the chronic study as a function of treatment. It is evident that A11 and A19 both suppress food intake in a similar fashion and give a significant reduction in body weight after 5 weeks of treatment, however, there is no difference between position A11 and A19 in terms on efficacy on body weight-.

### FIG. 18 Results, OGTT and Adipose Tissue

FIG. 18 shows the results from the OGTT with the corresponding iAUC (OGTT)(Figure A+B) as well as the weight of three different adipose tissue, epididymal, inguinal and perirenal (FIG. 18C-E) and the body weight as study end (FIG. 18F). Treatment with position A11 resulted in a significant reduction in iAUC (OGTT), epididymal WAT size, perirenal WAT size, and body weight as study. Treatment with position A19 resulted in a significant reduction in iAUC (OGTT), epididymal WAT size, and body weight as study, but not perirenal WAT size. Neither treatment significantly reduced the size of the inguinal WAT. Position A11 performed slightly better against vehicle compared to Position A19, but there was no significant difference between treatment groups.

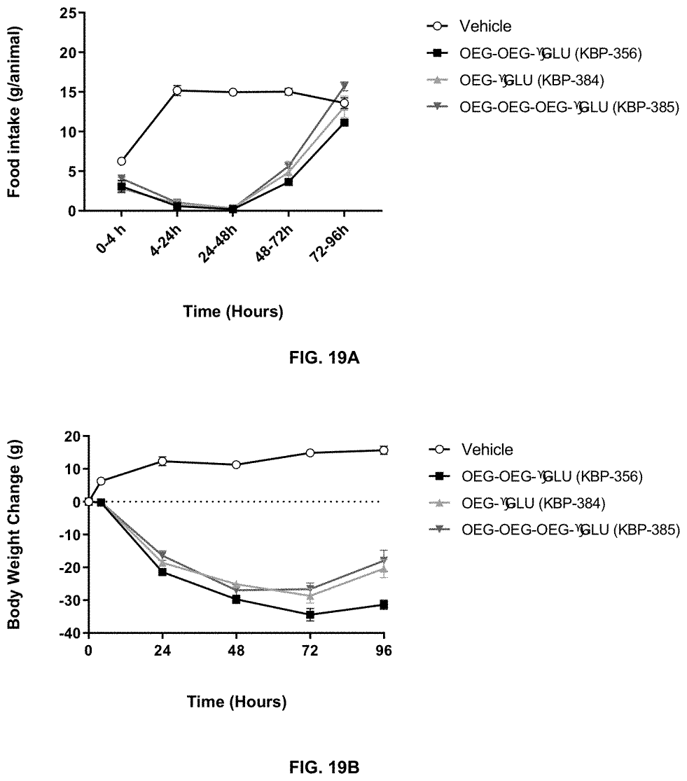

### Example 12 (FIG. 19)

Single dose comparative effect of three acylated linker variants (3, 7 and 8) at the same position and backbone, A11 and KBP-066, respectively, on food intake and body weight in an acute setting in 20-week old SD rats feed HFD for 8 weeks prior to the experiment.

| | | Acylation | Acylation | | Position/ |
|---|---|---|---|---|---|
| KBP | Core | length | Linker | Annotation | Acylation |
| KBP-356 | KBP-066 | C18 diacid | OEG- | KBP- | A11/3 |
| | | | OEG- | 066A11.03 | acylation |
| | | | γGLU | | |
| KBP-385 | KBP-066 | C18 diacid | OEG- | KBP- | A11/7 |
| | | | OEG- | 066A11.07 | acylation |
| | | | OEG- | | |
| | | | γGLU | | |
| KBP-384 | KBP-066 | C18 diacid | OEG- | KBP- | A11/8 |
| | | | γGLU | 066A11.08 | acylation |

Rats were single caged four days prior to the test. Rats were randomized by weight into eight groups (Vehicle (0.9% NaCl), KBPs (doses: 4 nmol/kg (\hat{ }13-14 μg/kg)). They were fasted overnight and then treated with a single dose of peptide or vehicle in the morning using subcutaneous administration. Food intake was monitored in the following intervals (0-4 hours, 4-24 hours, 24-48 hours, 48-72 hours, and 72-96 hours). Body weight was measured at baseline and at 4 hour, 24 hours, 48 hours, 72 hours and 96 hours post s.c injection.

### FIG. 19 Results—Food Intake and Body Weight

All three linkers tested worked well in an acute setting and all attenuated food intake (FIG. 19A) for up to 72 hours before rebounding. Acylation 3 was slightly better than 7 and 8 after 96 hours. This was evident on the corresponding body weight loss (FIG. 19B) where acylation 3 separate from acylation 7 and 8 early on (24 hours) and continued to separate on two following time points, 72 hours and 96 hours.

Furthermore, in terms of fibrillation potential (Table 4.4), acylation 8 appear to have some minor tendencies that could complicate further development of compound using that type of acylation.

### Summary of Results of Examples 7-12

### Acylations Length

The collected data from FIG. 11-14 and Table 4.1 suggest that the acylations, 3, 4 and 5, in the range of C18 diacid to C22 diacid, are interchangeable when developing acylated peptides for a once weekly dosing regimen in man.

Hence, C18, C20 and C22 diacid are the preferred length of acylation for this invention.

### Acylation Position

The collected data from FIGS. 15, 17 and 18 demonstrated that A19 may have a slight edge in an acute setting, whereas A11 has a slight edge in a chronic setting when using the KBP-066 backbone and 3 acylation.

Neither A11 nor A19 had any issues with fibrillation when combined with the KBP-066 backbone (Table 4.2)

Overall, these data suggest that the acylations position A11 and A19 together with KBP-066, are the two best all-round positions to acylate for development of a once weekly dosing regimen in man.

Hence A11 and A19 are the preferred positions for acylating KBP-066.

Similarly, based on Table 4.3 and FIG. 16 it is evident that A11 and A19 are the two best acylation positions when combined with KBP-021.

However, as A19 is very fibrillation prone in this setting, the A11 with 3 acylation is the preferred acylation position and -length for the KBP-021 backbone based on overall performance.

### Acylation Linker

Based on this test and Table 4.4 it appears that the OEG-OEG-γGLU linker is the optimal linker as shortening it generates potential fibrillation issues and elongating it at best does nothing. Furthermore, As FIG. 19 demonstrated, the OEG-OEG-γGLU linker was also the best performer in an acute setting, in vivo, making it the overall preferred acylation linker.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference.

1. A calcitonin mimetic that is a peptide acylated at a lysine residue located at the 11 position of the calcitonin mimetic and/or that is acylated at a lysine residue located at the 19 position of the calcitonin mimetic, wherein the side chain C-amino group of said lysine residue is acylated with an acyl group that is:
a C₁₆ or longer fatty acid with an optional linker, or
a C₁₆ or longer fatty diacid with an optional linker.

2. The calcitonin mimetic of claim 1, wherein the calcitonin mimetic is a peptide of formula (I) (a):
CX₂X₃LSTCX₈LGK_{Ac}
wherein
X₂=A, G or S,
X₃=N or S,
X₈=M, V or α-aminoisobutyric acid (AiB), and
wherein K_{Ac} is a lysine residue wherein the side chain ε-amino group is acylated with an acyl group that is:
the C₁₆ or longer fatty acid with an optional linker, or
the C₁₆ or longer fatty diacid with an optional linker.

3. The calcitonin mimetic of claim 1, wherein the calcitonin mimetic is peptide of formula (I) (b):
CX₂X₃LSTCX₈LGX₁₁X₁₂X₁₃X₁₄X₁₅X₁₆X₁₇X₁₈K_{Ac}
wherein
X₂=A, G or S,
X₃=N or S,
X₈=M, V or α-aminoisobutyric acid (AiB),
X₁₁=R, K, T, A or KAc,
X₁₂=L or Y,
X₁₃=S, T, W or Y,
X₁₄=Q, K, R or A,
X₁₅=D, E or N,
X₁₆=L or F,
X₁₇=H or N,
X₁₈=R, K or N, and
wherein K_{Ac} is a lysine residue wherein the side chain ε-amino group is acylated with an acyl group that is:
the C₁₆ or longer fatty acid with an optional linker, or
the C₁₆ or longer fatty diacid with an optional linker.

4. The calcitonin mimetic of claim 1, wherein the calcitonin mimetic is a peptide of formula (II):
CX₂X₃LSTCX₈LGX₁₁X₁₂X₁₃X₁₄X₁₅X₁₆X₁₇X₁₈X₁₉X₂₀X₂₁X₂₂X₂₃X₂₄X₂₅X₂₆X₂₇GX₂₉X₃₀X₃₁P
wherein
X₂=A, G or S,
X₃=N or S,
X₈=M, V or α-aminoisobutyric acid (AiB),
X₁₁=R, K, T, A or K_{Ac},
X₁₂=L or Y,
X₁₃=S, T, W or Y,
X₁₄=Q, K, R or A,
X₁₅=D, E or N,
X₁₆=L or F,
X₁₇=H or N,
X₁₈=R, K or N,
X₁₉=K_{Ac}, L, F or K,
X₂₀=Q, H or A,
X₂₁=T or R,
X₂₂=Y or F,
X₂₃=S or P,
X₂₄=G, K, Q or R,
X₂₅=T, I or M,
X₂₆=S, N, D, G or A,
X₂₇=T, V, F or I,
X₂₉=S, A, P or V,
X₃₀=N, G or E,
X₃₁=A, T or S,
wherein either X₁₁ is K_{Ac} and/or X₁₉ is K_{Ac}, and
wherein K_{Ac} is a lysine residue wherein the side chain ε-amino group is acylated with an acyl group that is:
a C₁₆ or longer fatty acid,
a C₁₆ or longer fatty diacid,
a linker-C₁₆ or longer fatty acid, or
a linker-C₁₆ or longer fatty diacid.

5. The calcitonin mimetic of claim 4, wherein the peptide of formula (II) is:
CX₂X₃LSTCX₈LGX₁₁LX₁₃X₁₄X₁₅LX₁₇X₁₈X₁₉X₂₀TX₂₂PX₂₄TDVGANAP
wherein
X₂=A, G or S,
X₃=N or S,
X₈=M, V or AiB,
X₁₁=KAc, R, K, T or A,
X₁₃=T, S or Y,
X₁₄=Q or A,
X₁₅=D or E,
X₁₇=H or N,
X₁₈=R or K,
X₁₉=KAc, L, F or K,
X₂₀=Q, H or A,
X₂₂=Y or F, or
X₂₄=K, Q or R.

6. The calcitonin mimetic of claim 4, wherein X₂ is S and X₃ is N; or X₂ is G and X₃ is N; or X₂ is A and X₃ is S.

7. The calcitonin mimetic of claim 4, wherein
X₁₁ is K_{Ac}, X₁₇ is H, X₁₈ is K, X₁₉ is L and X₂₀ is Q or A; or
X₁₁ is K_{Ac}, X₁₇ is H, X₁₈ is R, X₁₉ is L and X₂₀ is Q or A; or
X₁₁ is K_{Ac}, X₁₇ is N, X₁₈ is K, X₁₉ is F and X₂₀ is H or A; or
X₁₁ is K_{Ac}, X₁₇ is N, X₁₈ is R, X₁₉ is is F and X₂₀ is H or A; or
X₁₁ is K_{Ac}, X₁₇ is H, X₁₈ is K, X₁₉ is is K_{Ac} and X₂₀ is Q or A; or
X₁₁ is K_{Ac}, X₁₇ is H, X₁₈ is R, X₁₉ is is K_{Ac} and X₂₀ is Q or A; or
X₁₁ is K_{Ac}, X₁₇ is N, X₁₈ is K, X₁₉ is K_{Ac} and X₂₀ is H or A; or
X₁₁ is K_{Ac}, X₁₇ is N, X₁₈ is R, X₁₉ is K_{Ac} and X₂₀ is H or A.

8. The calcitonin mimetic claim 4, wherein
X₂ is S, X₃ is N, X₁₁ is K_{Ac}, X₁₃ is S, X₁₇ is H, X₁₈ is K or R, X₁₉ is L, X₂₀ is Q or A and X₂₂ is Y; or
X₂ is S, X₃ is N, X₁₁ is R or K, X₁₃ is S, X₁₇ is H, X₁₈ is K or R, X₁₉ is K_{Ac}, X₂₀ is Q or A and X₂₂ is Y; or
X₂ is A, X₃ is S, X₁₁ is K_{Ac} X₁₃ is S, X₁₇ is H, X₁₈ is K or R, X₁₉ is L, X₂₀ is Q or A and X₂₂ is F; or
X₂ is A, X₃ is S, X₁₁ is K_{Ac}, X₁₃ is S, X₁₇ is H, X₁₈ is K or R, X₁₉ is K_{Ac}, X₂₀ is Q or A and X₂₂ is F; or
X₂ is G, X₃ is N, X₁₁ is K_{Ac}, X₁₃ is T, X₁₇ is N, X₁₈ is K or R, X₁₉ is F, X₂₀ is H or A and X₂₂ is F; or
X₂ is G, X₃ is N, X₁₁ is R or K, X₁₃ is T, X₁₇ is N, X₁₈ is K or R, X₁₉ is K_{Ac}, X₂₀ is H or A and X₂₂ is F.

9-10. (canceled)

11. The calcitonin mimetic of claim 1, wherein the calcitonin mimetic is a 33mer peptide in accordance with formula (III):
CSNLSTCX₆LGX₇LSQDLHRX₈QTYPKX₁TX₅VGANAP; or
wherein the calcitonin mimetic is a 35mer peptide in accordance with formula (IV):
CSNLSTCX₆LGX₇LSQDLHRX₈QTYPKX₁X₂X₃TX₅VGANAP; or
wherein the calcitonin mimetic is a 36mer peptide in accordance with formula (V):
CSNLSTCX₆LGX₇LSQDLHRX₈QTYPKX₁X₂X₃X₄TX₅VGANAP; or
wherein the calcitonin mimetic is a 37mer peptide in accordance with formula (VI):
CSNLSTCX₆LGK_{Ac}LZX₁X₂X₃X₄TX₅VGANAP;
wherein each of X₁ to X₄ is any amino acid, with the proviso that at least one of X₁ to X₄ is a basic amino acid residue, and/or at least two of X₁ to X₄ are independently a polar amino acid residue or a basic amino acid residue, and/or at least one of X₁ to X₄ is a Gly residue, and wherein none of X₁ to X₄ is an acidic residue;
wherein X₅ is D or N;
wherein X₆ is AiB or M;
wherein either X₇ is K_{Ac} and X₈ is L, or X₇ is R or K and X₈ is K_{Ac};
wherein Z is selected from SQDLHRLSNNFGA, SQDLHRLQTYGAI or ANFLVHSSNNFGA; and
wherein K_{Ac} is a lysine residue wherein the side chain ε-amino group is acylated with an acyl group that is:
a C₁₆ or longer fatty acid,
a C₁₆ or longer fatty diacid,
a linker-C₁₆ or longer fatty acid, or
a linker-C₁₆ or longer fatty diacid.

12. The calcitonin mimetic of claim 11, wherein at least one of X₁ or X₄ is a basic amino acid residue.

13. The calcitonin mimetic of claim 11, wherein at least one of X₇ or X₄ is a basic amino acid residue, and at least two of X₁ to X₄ are independently a polar amino acid residue or a basic amino acid residue, and none of X₁ to X₄ is an acidic residue.

14-16. (canceled)

17. The calcitonin mimetic of claim 11, wherein the basic amino acid residue is Arg, His or Lys, and/or the polar amino acid residue is Ser, Thr, Asn, Gln or Cys.

18. The calcitonin mimetic of claim 11, wherein X₁ is selected from Asn, Phe, Val, Gly, lie, Leu, Lys, His or Arg;
X₂ is Ala, Asn, His, Leu, Ser, Thr, Gly or Lys;
X₃ is Ala, Phe, lie, Ser, Pro, Thr, Gly or Lys; and/or
X₄ is Ile, Leu, Gly, His, Arg, Asn, Ser, Lys, Thr or Gln;
with the proviso that at least one of X₁ or X₄ is a basic amino acid residue, and/or at least two of X₁ to X₄ are independently a polar amino acid residue and/or a basic amino acid residue, and/or at least one of X₁ to X₄ is a Gly residue.

19. The calcitonin mimetic of claim 18, wherein X₁ is Asn, Gly, lie, His or Arg;
X₂ is Asn, Leu, Thr, Gly or Lys;
X₃ is from Phe, Pro, lie, Ser, Thr, Gly or Lys; and/or
X₄ is from Gly, His, Asn, Ser, Lys, Thr or Gln.

20-21. (canceled)

22. The calcitonin mimetic of claim 1, wherein the linker comprises a glutamic acid residue and/or an oligoethyleneglycol (OEG) amino acid linker comprising one OEG amino acid or two or more OEG amino acids linked together, wherein said OEG amino acid is: 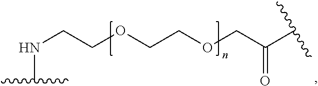
wherein n is from 1 to 10.

23. (canceled)

24. The calcitonin mimetic of claim 22, wherein said OEG amino acid linker further comprises one or more glutamic acid residues linked to the amino terminus or to the carboxyl terminus of the OEG amino acid linker.

25. (canceled)

26. The calcitonin mimetic of claim 22, wherein the OEG amino acid linker is

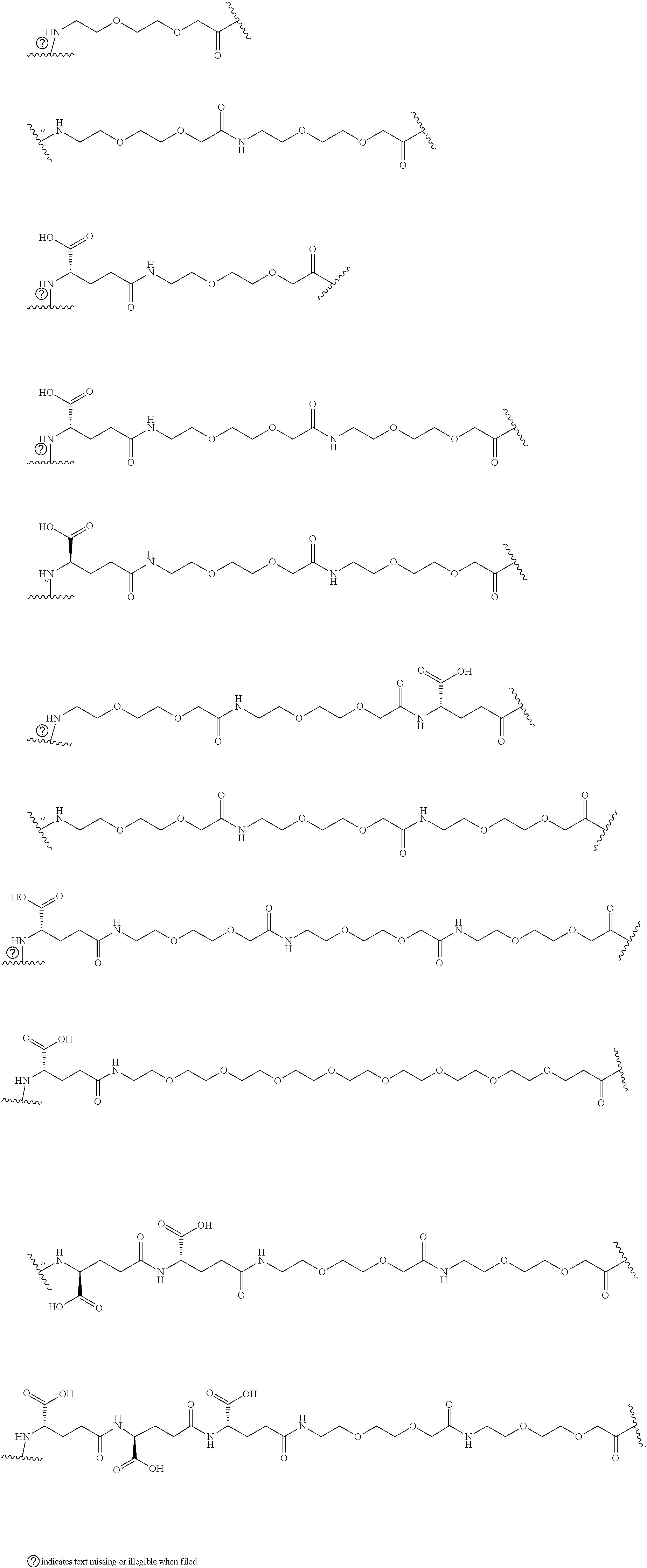

27. The calcitonin mimetic of claim 26, wherein the linker is

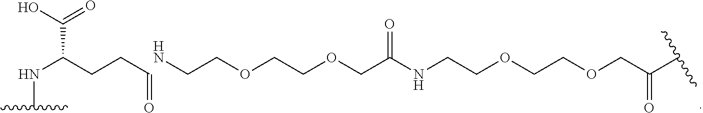

28. (canceled)

29. The calcitonin mimetic of claim 1, wherein the acyl group is a C₁₈ to C₃₀ fatty acid, a C₁₈ to C₃₀ fatty diacid, a linker-C₁₈ to C₃₀ fatty acid, or a linker-C₁₈ to C₃₀ fatty diacid.

30-31. (canceled)

32. The calcitonin mimetic of claim 1, wherein the peptide is:
CSNLSTCMLGK_{Ac}LSQDLHRLQTYPKTDVGANAP,
CSNLSTC(AiB)LGK_{Ac}LSQDLHRLQTYPKTDVGANAP,
CGNLSTC(AiB)LGK_{Ac}LTQDLNKFHTFPKTDVGANAP,
CSNLSTCVLGK_{Ac}LSQELHKLQTYPRTDVGANAP,
CSNLSTCMLGK_{Ac}LSQELHRLQTYPKTDVGANAP,
CASLSTCVLGK_{Ac}LSQDLHKLQTFPKTDVGANAP,
CASLSTCMLGK_{Ac}LSQDLHKLQTFPKTDVGANAP,
CGNLSTCMLGK_{Ac}LSQDLNKFHTFPQTDVGANAP,
CSNLSTC(AiB)LGK_{Ac}LANFLVHSSNNFGAILPKTDVGANAP,
CSNLSTCMLGK_{Ac}LSQDLHRLQTYPKHTDVGANAP,
CSNLSTCMLGK_{Ac}LSQDLHRLQTYPKHSSTDVGANAP,
CSNLSTCMLGK_{Ac}LSQDLHRLQTYPKHSSNTDVGANAP,
CSNLSTCMLGK_{Ac}LSQDLHRLSNNFGAILSSTNVGANAP,
CSNLSTCMLGK_{Ac}LSQDLHRLQTYGAILSPKTDVGANAP,
CSNLSTCMLGK_{Ac}LANFLVHSSNNFGAILPKTDVGANAP,
CSNLSTCMLGK_{Ac}LSQDLHRLQTYPKILSSTDVGANAP,
CSNLSTCMLGK_{Ac}LSQDLHRLQTYPKGLITTDVGANAP,
CSNLSTCMLGK_{Ac}LSQDLHRLQTYPKNNFGTDVGANAP,
CSNLSTCMLGK_{Ac}LSQDLHRLQTYPKRTTQTDVGANAP,
CSNLSTCMLGK_{Ac}LSQDLHRLQTYPKHTTNTDVGANAP,
CSNLSTCMLGK_{Ac}LSQDLHRLQTYPKHGGQTDVGANAP,
CSNLSTCMLGK_{Ac}LSQDLHRLQTYPKHKKNTDVGANAP,
CSNLSTCMLGK_{Ac}LSQDLHRLQTYPKHKKHTDVGANAP,
CSNLSTC(AiB)LGRLSQDLHRK_{Ac}QTYPKTDVGANAP,
or
CSNLSTCMLGRLSQELHRK_{Ac}QTYPKTDVGANAP

33. The calcitonin mimetic of claim 1, wherein the peptide is:
AcCSNLSTCMLGK_{Ac}LSQDLHRLQTYPKTDVGANAP-NH₂,
AcCSNLSTC(AiB)LGK_{Ac}LSQDLHRLQTYPKTDVGANAP-NH₂,
AcCGNLSTC(AiB)LGK_{Ac}LTQDLNKFHTFPKTDVGANAP-NH₂,
AcCSNLSTCVLGK_{Ac}LSQELHKLQTYPRTDVGANAP-NH₂,
AcCSNLSTCMLGK_{Ac}LSQELHRLQTYPKTDVGANAP-NH₂,
AcCASLSTCVLGK_{Ac}LSQDLHKLQTFPKTDVGANAP-NH₂,
AcCASLSTCMLGK_{Ac}LSQDLHKLQTFPKTDVGANAP-H2,
AcCGNLSTCMLGK_{Ac}LSQDLNKFHTFPQTDVGANAP-NH21
AcCSNLSTC(AiB)LGK_{Ac}LANFLVHSSNNFGAILPKTDVGANAP-NH₂,
AcCSNLSTCMLGK_{Ac}LSQDLHRLQTYPKHTDVGANAP-NH₂,
AcCSNLSTCMLGK_{Ac}LSQDLHRLQTYPKHSSTDVGANAP-NH₂,
AcCSNLSTCMLGK_{Ac}LSQDLHRLQTYPKHSSNTDVGANAP-NH₂,
AcCSNLSTCMLGK_{Ac}LSQDLHRLSNNFGAILSSTNVGANAP-NH₂,
AcCSNLSTCMLGK_{Ac}LSQDLHRLQTYGAILSPKTDVGANAP-NH₂,
AcCSNLSTCMLGK_{Ac}LANFLVHSSNNFGAILPKTDVGANAP-NH₂,
AcCSNLSTCMLGK_{Ac}LSQDLHRLQTYPKILSSTDVGANAP-NH₂,
AcCSNLSTCMLGK_{Ac}LSQDLHRLQTYPKGLITTDVGANAP-NH₂,
AcCSNLSTCMLGK_{Ac}LSQDLHRLQTYPKNNFGTDVGANAP-NH₂,
AcCSNLSTCMLGK_{Ac}LSQDLHRLQTYPKRTTQTDVGANAP-NH₂,
AcCSNLSTCMLGK_{Ac}LSQDLHRLQTYPKHTTNTDVGANAP-NH₂,
AcCSNLSTCMLGK_{Ac}LSQDLHRLQTYPKHGGQTDVGANAP-NH₂,
AcCSNLSTCMLGK_{Ac}LSQDLHRLQTYPKHKKNTDVGANAP-NH₂,
AcCSNLSTCMLGK_{Ac}LSQDLHRLQTYPKHKKHTDVGANAP-NH₂,
AcCSNLSTC(AiB)LGRLSQDLHRK_{Ac}QTYPKTDVGANAP-NH₂,
or
AcCSNLSTCMLGK_{Ac}LSQELHRLQTYPKTDVGANAP-NH₂,
and
wherein K_{Ac} is acylated with a linker-fatty diacid, wherein the fatty diacid is a C₁₈ to C₂₂ fatty diacid and the linker is

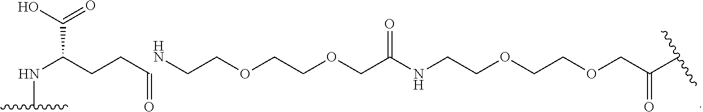

34. The calcitonin mimetic of claim 1, wherein the peptide is formulated for enteral administration, for oral administration, for parenteral administration, or for injection.

35. The calcitonin mimetic of claim 34, wherein the peptide is is coated with citric acid particles for oral administration, and wherein the coated citric acid particles increase the oral bioavailability of the peptide.

36. A pharmaceutical composition comprising the calcitonin mimetic of claim 1, and a pharmaceutically acceptable carrier.

37. The pharmaceutical composition of claim 36, wherein the pharmaceutically acceptable carrier comprises N-(5-chlorosalicyloyl)-8-aminocaprylic acid (5-CNAC), sodium salt of 10-(2-Hydroxybenzamido)decanoic acid (SNAD), or sodium salt of N-(8-[2-hydroxybenzoyl]amino)caprylic acid (SNAC) for oral administration.

38-45. (canceled)

46. The calcitonin mimetic of claim 36, further comprising an insulin sensitizer or a weight loss drug.

47. (canceled)

48. A method of treating diabetes (Type I and/or Type II), excess bodyweight, excessive food consumption, metabolic syndrome, rheumatoid arthritis, non-alcoholic steatohepatitis (NASH), non-alcoholic fatty liver disease, alcoholic fatty liver disease, osteoporosis, or osteoarthritis, poorly regulated blood glucose levels, poorly regulated response to glucose tolerance tests, or poor regulation of food intake, comprising administering an effective amount of the calcitonin mimetic of claim 1 to a patient in need of said treatment.

49. A method of treating diabetes (Type I and/or Type II), excess bodyweight, excessive food consumption, metabolic syndrome, rheumatoid arthritis, non-alcoholic steatohepatitis (NASH), non-alcoholic fatty liver disease, alcoholic fatty liver disease, osteoporosis, or osteoarthritis, poorly regulated blood glucose levels, poorly regulated response to glucose tolerance tests, or poor regulation of food intake, comprising administering an effective amount of the calcitonin mimetic of claim 1 in combination with metformin or another insulin sensitizer to a patient in need of said treatment.

50. A method of treating an overweight condition comprising administering an effective amount of the calcitonin mimetic of claim 1 in combination with a weight loss drug to a patient in need of said treatment.


SEQUENCE LISTING (189 sequences)

SEQ ID NO: 1 - protein, 32 aa, Salmo salar
CSNLSTCVLGKLSQELHKLQTYPRTNTGSGTP

SEQ ID NO: 2 - protein, 32 aa, Unknown
  Description of Unknown Eel calcitonin sequence
CSNLSTCVLGKLSQELHKLQTYPRTDVGAGTP

SEQ ID NO: 3 - protein, 32 aa, Gallus gallus
CASLSTCVLGKLSQELHKLQTYPRTDVGAGTP

SEQ ID NO: 4 - protein, 32 aa, Mus sp.
CGNLSTCMLGTYTQDLNKFHTFPQTSIGVEAP

SEQ ID NO: 5 - protein, 32 aa, Rattus sp.
CGNLSTCMLGTYTQDLNKFHTFPQTSIGVGAP

SEQ ID NO: 6 - protein, 32 aa, Equus caballus
CSNLSTCVLGTYTQDLNKFHTFPQTAIGVGAP

SEQ ID NO: 7 - protein, 32 aa, Unknown
  Description of Unknown Canine calcitonin sequence
CSNLSTCVLGTYSKDLNNFHTFSGIGFGAETP

SEQ ID NO: 8 - protein, 32 aa, Unknown; identical to SEQ ID NO: 6
  Description of Unknown Canine calcitonin sequence

SEQ ID NO: 9 - protein, 32 aa, Sus scrofa
CSNLSTCVLSAYWRNLNNFHRFSGMGFGPETP

SEQ ID NO: 10 - protein, 32 aa, Homo sapiens
CGNLSTCMLGTYTQDFNKFHTFPQTAIGVGAP

SEQ ID NO: 11 - protein, 11 aa, Artificial Sequence
  Description of Artificial Sequence Synthetic peptide
  VARIANT (2)..(2): /replace=“Gly” or “Ser”
  VARIANT (3)..(3): /replace=“Ser”
  VARIANT (8)..(8): /replace=“Val” or “AiB”
  MOD_RES (11)..(11): Lys-Ac
  SITE (1)..(11): /note=“Variant residues given in the sequence have no preference with respect to those in the annotations for variant positions”
  source: /note=“See specification as filed for detailed description of substitutions and preferred embodiments”
CANLSTCMLGX

SEQ ID NO: 12 - protein, 19 aa, Artificial Sequence
  Description of Artificial Sequence Synthetic peptide
  VARIANT (2)..(2): /replace=“Gly” or “Ser”
  VARIANT (3)..(3): /replace=“Ser”
  VARIANT (8)..(8): /replace=“Val” or “AiB”
  VARIANT (11)..(11): /replace=“Lys” or “Thr” or “Ala” or “Lys-Ac”
  VARIANT (12)..(12): /replace=“Tyr”
  VARIANT (13)..(13): /replace=“Thr” or “Trp” or “Tyr”
  VARIANT (14)..(14): /replace=“Lys” or “Arg” or “Ala”
  VARIANT (15)..(15): /replace=“Glu” or “Asn”
  VARIANT (16)..(16): /replace=“Phe”
  VARIANT (17)..(17): /replace=“Asn”
  VARIANT (18)..(18): /replace=“Lys” or “Asn”
  MOD_RES (19)..(19): Lys-Ac
  SITE (1)..(19): /note=“Variant residues given in the sequence have no preference with respect to those in the annotations for variant positions”
  source: /note=“See specification as filed for detailed description of substitutions and preferred embodiments”
CANLSTCMLGRLSQDLHRX

SEQ ID NO: 13 - protein, 32 aa, Artificial Sequence
  Description of Artificial Sequence Synthetic polypeptide
  VARIANT (2)..(2): /replace=“Gly” or “Ser”
  VARIANT (3)..(3): /replace=“Ser”
  VARIANT (8)..(8): /replace=“Val” or “AiB”
  VARIANT (11)..(11): /replace=“Lys-Ac” or “Lys” or “Thr” or “Ala”
  VARIANT (12)..(12): /replace=“Tyr”
  VARIANT (13)..(13): /replace=“Thr” or “Trp” or “Tyr”
  VARIANT (14)..(14): /replace=“Lys” or “Arg” or “Ala”
  VARIANT (15)..(15): /replace=“Glu” or “Asn”
  VARIANT (16)..(16): /replace=“Phe”
  VARIANT (17)..(17): /replace=“Asn”
  VARIANT (18)..(18): /replace=“Lys” or “Asn”
  VARIANT (19)..(19): /replace=“Lys-Ac” or “Phe” or “Lys”
  VARIANT (20)..(20): /replace=“His” or “Ala”
  VARIANT (21)..(21): /replace=“Arg”
  VARIANT (22)..(22): /replace=“Phe”
  VARIANT (23)..(23): /replace=“Pro”
  VARIANT (24)..(24): /replace=“Lys” or “Gln” or “Arg”
  VARIANT (25)..(25): /replace=“Ile” or “Met”
  VARIANT (26)..(26): /replace=“Asn” or “Asp” or “Gly” or “Ala”
  VARIANT (27)..(27): /replace=“Val” or “Phe” or “Ile”
  VARIANT (29)..(29): /replace=“Ala” or “Pro” or “Val”
  VARIANT (30)..(30): /replace=“Gly” or “Glu”
  VARIANT (31)..(31): /replace=“Thr” or “Ser”
  SITE (1)..(32): /note=“Variant residues given in the sequence have no preference with respect to those in the annotations for variant positions”
  source: /note=“See specification as filed for detailed description of substitutions and preferred embodiments”
CANLSTCMLGRLSQDLHRLQTYSGTSTGSNAP

SEQ ID NO: 14 - protein, 32 aa, Artificial Sequence
  Description of Artificial Sequence Synthetic polypeptide
  VARIANT (2)..(2): /replace=“Gly” or “Ser”
  VARIANT (3)..(3): /replace=“Ser”
  VARIANT (8)..(8): /replace=“Val” or “AiB”
  VARIANT (11)..(11): /replace=“Lys-Ac” or “Lys” or “Thr” or “Ala”
  VARIANT (13)..(13): /replace=“Ser” or “Tyr”
  VARIANT (14)..(14): /replace=“Ala”
  VARIANT (15)..(15): /replace=“Glu”
  VARIANT (17)..(17): /replace=“Asn”
  VARIANT (18)..(18): /replace=“Lys”
  VARIANT (19)..(19): /replace=“Lys-Ac” or “Phe” or “Lys”
  VARIANT (20)..(20): /replace=“His” or “Ala”
  VARIANT (22)..(22): /replace=“Phe”
  VARIANT (24)..(24): /replace=“Gln” or “Arg”
  SITE (1)..(32): /note=“Variant residues given in the sequence have no preference with respect to those in the annotations for variant positions”
  source: /note=“See specification as filed for detailed description of substitutions and preferred embodiments”
CANLSTCMLGRLTQDLHRLQTYPKTDVGANAP

SEQ ID NO: 15 - protein, 33 aa, Artificial Sequence
  Description of Artificial Sequence Synthetic polypeptide
  VARIANT (8)..(8): /replace=“AiB”
  VARIANT (11)..(11): /replace=“Lys-Ac” or “Lys”
  VARIANT (19)..(19): /replace=“Lys-Ac”
  MOD_RES (25)..(25): Any amino acid
  VARIANT (27)..(27): /replace=“Asn”
  SITE (1)..(33): /note=“Variant residues given in the sequence have no preference with respect to those in the annotations for variant positions”
  source: /note=“See specification as filed for detailed description of substitutions and preferred embodiments”
CSNLSTCMLGRLSQDLHRLQTYPKXTDVGANAP

SEQ ID NO: 16 - protein, 35 aa, Artificial Sequence
  Description of Artificial Sequence Synthetic polypeptide
  VARIANT (8)..(8): /replace=“AiB”
  VARIANT (11)..(11): /replace=“Lys-Ac” or “Lys”
  VARIANT (19)..(19): /replace=“Lys-Ac”
  MOD_RES (25)..(25): Any amino acid
  MOD_RES (26)..(26): Any amino acid
  MOD_RES (27)..(27): Any amino acid
  VARIANT (29)..(29): /replace=“Asn”
  SITE (1)..(35): /note=“Variant residues given in the sequence have no preference with respect to those in the annotations for variant positions”
  source: /note=“See specification as filed for detailed description of substitutions and preferred embodiments”
CSNLSTCMLGRLSQDLHRLQTYPKXXXTDVGANAP

SEQ ID NO: 17 - protein, 36 aa, Artificial Sequence
  Description of Artificial Sequence Synthetic polypeptide
  VARIANT (8)..(8): /replace=“AiB”
  VARIANT (11)..(11): /replace=“Lys-Ac” or “Lys”
  VARIANT (19)..(19): /replace=“Lys-Ac”
  MOD_RES (25)..(25): Any amino acid
  MOD_RES (26)..(26): Any amino acid
  MOD_RES (27)..(27): Any amino acid
  MOD_RES (28)..(28): Any amino acid
  VARIANT (30)..(30): /replace=“Asn”
  SITE (1)..(36): /note=“Variant residues given in the sequence have no preference with respect to those in the annotations for variant positions”
  source: /note=“See specification as filed for detailed description of substitutions and preferred embodiments”
CSNLSTCMLGRLSQDLHRLQTYPKXXXXTDVGANAP

SEQ ID NO: 18 - protein, 37 aa, Artificial Sequence
  Description of Artificial Sequence Synthetic polypeptide
  VARIANT (8)..(8): /replace=“AiB”
  MOD_RES (11)..(11): Lys-Ac
  SITE (13)..(25): /note=“This region may encompass one of the following sequences SQDLHRLSNNFGA, SQDLHRLQTYGAI, or ANFLVHSSNNFGA”
  MOD_RES (26)..(26): Any amino acid
  MOD_RES (27)..(27): Any amino acid
  MOD_RES (28)..(28): Any amino acid
  MOD_RES (29)..(29): Any amino acid
  VARIANT (31)..(31): /replace=“Asn”
  SITE (1)..(37): /note=“Variant residues given in the sequence have no preference with respect to those in the annotations for variant positions”
  source: /note=“See specification as filed for detailed description of substitutions and preferred embodiments”
CSNLSTCMLGXLXXXXXXXXXXXXXXXXXTDVGANAP

SEQ ID NO: 19 - protein, 13 aa, Artificial Sequence
  Description of Artificial Sequence Synthetic peptide
SQDLHRLSNNFGA

SEQ ID NO: 20 - protein, 13 aa, Artificial Sequence
  Description of Artificial Sequence Synthetic peptide
SQDLHRLQTYGAI

SEQ ID NO: 21 - protein, 13 aa, Artificial Sequence
  Description of Artificial Sequence Synthetic peptide
ANFLVHSSNNFGA

SEQ ID NO: 22 - protein, 13 aa, Artificial Sequence
  Description of Artificial Sequence Synthetic peptide
SQDLHRLSNNFGA

SEQ ID NO: 23 - protein, 13 aa, Artificial Sequence
  Description of Artificial Sequence Synthetic peptide
SQDLHRLQTYGAI

SEQ ID NO: 24 - protein, 32 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKTDVGANAP

SEQ ID NO: 25 - protein, 32 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQELHRLQTYPKTDVGANAP

SEQ ID NO: 26 - protein, 32 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCVLGXLSQELHKLQTYPRTDVGANAP

SEQ ID NO: 27 - protein, 32 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CASLSTCVLGXLSQDLHKLQTFPKTDVGANAP

SEQ ID NO: 28 - protein, 32 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CGNLSTCMLGXLSQDLNKFHTFPQTDVGANAP

SEQ ID NO: 29 - protein, 32 aa, Artificial Sequence
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”
CSNLSTCXLGXLSQDLHRLQTYPKTDVGANAP

SEQ ID NO: 30 - protein, 32 aa, Artificial Sequence
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”
CGNLSTCXLGXLTQDLNKFHTFPKTDVGANAP

SEQ ID NO: 31 - protein, 37 aa, Artificial Sequence
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”
CSNLSTCXLGXLANFLVHSSNNFGAILPKTDVGANAP

SEQ ID NO: 32 - protein, 33 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKHTDVGANAP

SEQ ID NO: 33 - protein, 35 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKHSSTDVGANAP

SEQ ID NO: 34 - protein, 36 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKHSSNTDVGANAP

SEQ ID NO: 35 - protein, 37 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLSNNFGAILSSTNVGANAP

SEQ ID NO: 36 - protein, 37 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYGAILSPKTDVGANAP

SEQ ID NO: 37 - protein, 37 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLANFLVHSSNNFGAILPKTDVGANAP

SEQ ID NO: 38 - protein, 36 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKILSSTDVGANAP

SEQ ID NO: 39 - protein, 36 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKGLITTDVGANAP

SEQ ID NO: 40 - protein, 36 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKNNFGTDVGANAP

SEQ ID NO: 41 - protein, 36 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKRTTQTDVGANAP

SEQ ID NO: 42 - protein, 36 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKHTTNTDVGANAP

SEQ ID NO: 43 - protein, 36 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKHGGQTDVGANAP

SEQ ID NO: 44 - protein, 36 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKHKKNTDVGANAP

SEQ ID NO: 45 - protein, 36 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CSNLSTCMLGXLSQDLHRLQTYPKHKKHTDVGANAP

SEQ ID NO: 46 - protein, 32 aa, Artificial Sequence
  Description of Artificial Sequence Synthetic polypeptide
  MOD_RES (8)..(8): AiB
  MOD_RES (19)..(19): Lys-Ac
  source: /note=“See specification as filed for detailed description of substitutions and preferred embodiments”
CSNLSTCXLGRLSQDLHRXQTYPKTDVGANAP

SEQ ID NO: 47 - protein, 32 aa, Artificial Sequence
  Description of Artificial Sequence Synthetic polypeptide
  MOD_RES (19)..(19): Lys-Ac
  source: /note=“See specification as filed for detailed description of substitutions and preferred embodiments”
CSNLSTCMLGRLSQELHRXQTYPKTDVGANAP

SEQ ID NO: 48 - protein, 32 aa, Artificial Sequence; identical to SEQ ID NO: 24
  Description of Artificial Sequence Synthetic polypeptide
  MOD_RES (11)..(11): Lys-Ac
  source: /note=“See specification as filed for detailed description of substitutions and preferred embodiments”

SEQ ID NO: 49 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”

SEQ ID NO: 50 - protein, 32 aa, Artificial Sequence; identical to SEQ ID NO: 30
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”

SEQ ID NO: 51 - protein, 32 aa, Artificial Sequence; identical to SEQ ID NO: 26
  Description of Artificial Sequence Synthetic polypeptide
  MOD_RES (11)..(11): Lys-Ac
  source: /note=“See specification as filed for detailed description of substitutions and preferred embodiments”

SEQ ID NO: 52 - protein, 32 aa, Artificial Sequence; identical to SEQ ID NO: 25
  Description of Artificial Sequence Synthetic polypeptide
  MOD_RES (11)..(11): Lys-Ac
  source: /note=“See specification as filed for detailed description of substitutions and preferred embodiments”

SEQ ID NO: 53 - protein, 32 aa, Artificial Sequence; identical to SEQ ID NO: 27
  Description of Artificial Sequence Synthetic polypeptide
  MOD_RES (11)..(11): Lys-Ac
  source: /note=“See specification as filed for detailed description of substitutions and preferred embodiments”

SEQ ID NO: 54 - protein, 32 aa, Artificial Sequence; identical to SEQ ID NO: 28
  Description of Artificial Sequence Synthetic polypeptide
  MOD_RES (11)..(11): Lys-Ac
  source: /note=“See specification as filed for detailed description of substitutions and preferred embodiments”

SEQ ID NO: 55 - protein, 33 aa, Artificial Sequence; identical to SEQ ID NO: 32
  Description of Artificial Sequence Synthetic polypeptide
  MOD_RES (11)..(11): Lys-Ac
  source: /note=“See specification as filed for detailed description of substitutions and preferred embodiments”

SEQ ID NO: 56 - protein, 35 aa, Artificial Sequence; identical to SEQ ID NO: 33
  Description of Artificial Sequence Synthetic polypeptide
  MOD_RES (11)..(11): Lys-Ac
  source: /note=“See specification as filed for detailed description of substitutions and preferred embodiments”

SEQ ID NO: 57 - protein, 36 aa, Artificial Sequence; identical to SEQ ID NO: 34
  Description of Artificial Sequence Synthetic polypeptide
  MOD_RES (11)..(11): Lys-Ac
  source: /note=“See specification as filed for detailed description of substitutions and preferred embodiments”

SEQ ID NO: 58 - protein, 37 aa, Artificial Sequence; identical to SEQ ID NO: 35
  Description of Artificial Sequence Synthetic polypeptide
  MOD_RES (11)..(11): Lys-Ac
  source: /note=“See specification as filed for detailed description of substitutions and preferred embodiments”

SEQ ID NO: 59 - protein, 37 aa, Artificial Sequence; identical to SEQ ID NO: 36
  Description of Artificial Sequence Synthetic polypeptide
  MOD_RES (11)..(11): Lys-Ac
  source: /note=“See specification as filed for detailed description of substitutions and preferred embodiments”

SEQ ID NO: 60 - protein, 37 aa, Artificial Sequence; identical to SEQ ID NO: 37
  Description of Artificial Sequence Synthetic polypeptide
  MOD_RES (11)..(11): Lys-Ac
  source: /note=“See specification as filed for detailed description of substitutions and preferred embodiments”

SEQ ID NO: 61 - protein, 36 aa, Artificial Sequence; identical to SEQ ID NO: 38
  Description of Artificial Sequence Synthetic polypeptide
  MOD_RES (11)..(11): Lys-Ac
  source: /note=“See specification as filed for detailed description of substitutions and preferred embodiments”

SEQ ID NO: 62 - protein, 36 aa, Artificial Sequence; identical to SEQ ID NO: 39
  Description of Artificial Sequence Synthetic polypeptide
  MOD_RES (11)..(11): Lys-Ac
  source: /note=“See specification as filed for detailed description of substitutions and preferred embodiments”

SEQ ID NO: 63 - protein, 36 aa, Artificial Sequence; identical to SEQ ID NO: 40
  Description of Artificial Sequence Synthetic polypeptide
  MOD_RES (11)..(11): Lys-Ac
  source: /note=“See specification as filed for detailed description of substitutions and preferred embodiments”

SEQ ID NO: 64 - protein, 36 aa, Artificial Sequence; identical to SEQ ID NO: 41
  Description of Artificial Sequence Synthetic polypeptide
  MOD_RES (11)..(11): Lys-Ac
  source: /note=“See specification as filed for detailed description of substitutions and preferred embodiments”

SEQ ID NO: 65 - protein, 36 aa, Artificial Sequence; identical to SEQ ID NO: 42
  Description of Artificial Sequence Synthetic polypeptide
  MOD_RES (11)..(11): Lys-Ac
  source: /note=“See specification as filed for detailed description of substitutions and preferred embodiments”

SEQ ID NO: 66 - protein, 36 aa, Artificial Sequence; identical to SEQ ID NO: 43
  Description of Artificial Sequence Synthetic polypeptide
  MOD_RES (11)..(11): Lys-Ac
  source: /note=“See specification as filed for detailed description of substitutions and preferred embodiments”

SEQ ID NO: 67 - protein, 36 aa, Artificial Sequence; identical to SEQ ID NO: 44
  Description of Artificial Sequence Synthetic polypeptide
  MOD_RES (11)..(11): Lys-Ac
  source: /note=“See specification as filed for detailed description of substitutions and preferred embodiments”

SEQ ID NO: 68 - protein, 36 aa, Artificial Sequence; identical to SEQ ID NO: 45
  Description of Artificial Sequence Synthetic polypeptide
  MOD_RES (11)..(11): Lys-Ac
  source: /note=“See specification as filed for detailed description of substitutions and preferred embodiments”

SEQ ID NO: 69 - protein, 37 aa, Artificial Sequence; identical to SEQ ID NO: 31
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”

SEQ ID NO: 70 - protein, 32 aa, Artificial Sequence; identical to SEQ ID NO: 46
  Description of Artificial Sequence Synthetic polypeptide
  MOD_RES (8)..(8): AiB
  MOD_RES (19)..(19): Lys-Ac
  source: /note=“See specification as filed for detailed description of substitutions and preferred embodiments”

SEQ ID NO: 71 - protein, 32 aa, Artificial Sequence; identical to SEQ ID NO: 47
  Description of Artificial Sequence Synthetic polypeptide
  MOD_RES (19)..(19): Lys-Ac
  source: /note=“See specification as filed for detailed description of substitutions and preferred embodiments”

SEQ ID NO: 72 - protein, 32 aa, Artificial Sequence
  Description of Artificial Sequence Synthetic polypeptide
CSNLSTCMLGRLSQDLHRLQTYPKTDVGANAP

SEQ ID NO: 73 - protein, 32 aa, Artificial Sequence
  Description of Artificial Sequence Synthetic polypeptide
  MOD_RES (8)..(8): AiB
CSNLSTCXLGRLSQDLHRLQTYPKTDVGANAP

SEQ ID NO: 74 - protein, 32 aa, Artificial Sequence
  Description of Artificial Sequence Synthetic polypeptide
  MOD_RES (8)..(8): AiB
CGNLSTCXLGRLTQDLNKFHTFPKTDVGANAP

SEQ ID NO: 75 - protein, 32 aa, Artificial Sequence
  Description of Artificial Sequence Synthetic polypeptide
CSNLSTCVLGKLSQELHKLQTYPRTDVGANAP

SEQ ID NO: 76 - protein, 37 aa, Artificial Sequence
  Description of Artificial Sequence Synthetic polypeptide
  MOD_RES (8)..(8): AiB
CSNLSTCXLGRLANFLVHSSNNFGAILPKTDVGANAP

SEQ ID NO: 77 - protein, 32 aa, Artificial Sequence
  Description of Artificial Sequence Synthetic polypeptide
CSNLSTCMLGRLSQELHRLQTYPKTDVGANAP

SEQ ID NO: 78 - protein, 32 aa, Artificial Sequence; identical to SEQ ID NO: 26
  Description of Artificial Sequence Synthetic polypeptide
  MOD_RES (11)..(11): Lys-Ac-(glutamic acid linker)-(C16 fatty acid [palmitate])
  source: /note=“See specification as filed for detailed description of substitutions and preferred embodiments”

SEQ ID NO: 79 - protein, 32 aa, Artificial Sequence
  Description of Artificial Sequence Synthetic polypeptide
  MOD_RES (18)..(18): Lys-Ac-(glutamic acid linker)-(C16 fatty acid [palmitate])
  source: /note=“See specification as filed for detailed description of substitutions and preferred embodiments”
CSNLSTCMLGRLSQDLHXLQTYPKTDVGANAP

SEQ ID NO: 80 - protein, 32 aa, Artificial Sequence; identical to SEQ ID NO: 24
  Description of Artificial Sequence Synthetic polypeptide
  MOD_RES (11)..(11): Lys-Ac-(glutamic acid linker)-(C16 fatty acid [palmitate])
  source: /note=“See specification as filed for detailed description of substitutions and preferred embodiments”

SEQ ID NO: 81 - protein, 32 aa, Artificial Sequence
  Description of Artificial Sequence Synthetic polypeptide
  MOD_RES (12)..(12): Lys-Ac-(glutamic acid linker)-(C16 fatty acid [palmitate])
  source: /note=“See specification as filed for detailed description of substitutions and preferred embodiments”
CSNLSTCMLGRXSQDLHRLQTYPKTDVGANAP

SEQ ID NO: 82 - protein, 32 aa, Artificial Sequence
  Description of Artificial Sequence Synthetic polypeptide
  MOD_RES (16)..(16): Lys-Ac-(glutamic acid linker)-(C16 fatty acid [palmitate])
  source: /note=“See specification as filed for detailed description of substitutions and preferred embodiments”
CSNLSTCMLGRLSQDXHRLQTYPKTDVGANAP

SEQ ID NO: 83 - protein, 32 aa, Artificial Sequence
  Description of Artificial Sequence Synthetic polypeptide
  MOD_RES (9)..(9): Lys-Ac-(glutamic acid linker)-(C16 fatty acid [palmitate])
  source: /note=“See specification as filed for detailed description of substitutions and preferred embodiments”
CSNLSTCMXGRLSQDLHRLQTYPKTDVGANAP

SEQ ID NO: 84 - protein, 34 aa, Artificial Sequence
  Description of Artificial Sequence Synthetic polypeptide
  MOD_RES (34)..(34): Lys-Ac-(glutamic acid linker)-(C16 fatty acid [palmitate])
  source: /note=“See specification as filed for detailed description of substitutions and preferred embodiments”
CSNLSTCMLGRLSQDLHRLQTYPKTDVGANAPKX

SEQ ID NO: 85 - protein, 32 aa, Artificial Sequence
  Description of Artificial Sequence Synthetic polypeptide
  MOD_RES (8)..(8): AiB
  MOD_RES (9)..(9): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCXXGRLSQDLHRLQTYPKTDVGANAP

SEQ ID NO: 86 - protein, 32 aa, Artificial Sequence; identical to SEQ ID NO: 85
  Description of Artificial Sequence Synthetic polypeptide
  MOD_RES (8)..(8): AiB
  MOD_RES (9)..(9): Lys-Ac-(glutamic acid linker)-(C18 diacid [Octadecanedioic acid])
  source: /note=“See specification as filed for detailed description of substitutions and preferred embodiments”

SEQ ID NO: 87 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 88 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(glutamic acid linker)-(C18 diacid [Octadecanedioic acid])
  source: /note=“See specification as filed for detailed description of substitutions and preferred embodiments”

SEQ ID NO: 89 - protein, 34 aa, Artificial Sequence
  Description of Artificial Sequence Synthetic polypeptide
  MOD_RES (8)..(8): AiB
  MOD_RES (34)..(34): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCXLGRLSQDLHRLQTYPKTDVGANAPKX

SEQ ID NO: 90 - protein, 34 aa, Artificial Sequence; identical to SEQ ID NO: 89
  Description of Artificial Sequence Synthetic polypeptide
  MOD_RES (8)..(8): AiB
  MOD_RES (34)..(34): Lys-Ac-(glutamic acid linker)-(C18 diacid [Octadecanedioic acid])
  source: /note=“See specification as filed for detailed description of substitutions and preferred embodiments”

SEQ ID NO: 91 - protein, 32 aa, Artificial Sequence
  Description of Artificial Sequence Synthetic polypeptide
  MOD_RES (8)..(8): AiB
  MOD_RES (9)..(9): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CGNLSTCXXGRLTQDLNKFHTFPKTDVGANAP

SEQ ID NO: 92 - protein, 32 aa, Artificial Sequence; identical to SEQ ID NO: 91
  Description of Artificial Sequence Synthetic polypeptide
  MOD_RES (8)..(8): AiB
  MOD_RES (9)..(9): Lys-Ac-(glutamic acid linker)-(C18 diacid [Octadecanedioic acid])
  source: /note=“See specification as filed for detailed description of substitutions and preferred embodiments”

SEQ ID NO: 93 - protein, 32 aa, Artificial Sequence; identical to SEQ ID NO: 30
  Description of Artificial Sequence Synthetic polypeptide
  MOD_RES (8)..(8): AiB
  MOD_RES (11)..(11): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 94 - protein, 32 aa, Artificial Sequence; identical to SEQ ID NO: 30
  Description of Artificial Sequence Synthetic polypeptide
  MOD_RES (8)..(8): AiB
  MOD_RES (11)..(11): Lys-Ac-(glutamic acid linker)-(C18 diacid [Octadecanedioic acid])
  source: /note=“See specification as filed for detailed description of substitutions and preferred embodiments”

SEQ ID NO: 95 - protein, 34 aa, Artificial Sequence
  Description of Artificial Sequence Synthetic polypeptide
  MOD_RES (8)..(8): AiB
  MOD_RES (34)..(34): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CGNLSTCXLGRLTQDLNKFHTFPKTDVGANAPKX

SEQ ID NO: 96 - protein, 34 aa, Artificial Sequence; identical to SEQ ID NO: 95
  Description of Artificial Sequence Synthetic polypeptide
  MOD_RES (8)..(8): AiB
  MOD_RES (34)..(34): Lys-Ac-(glutamic acid linker)-(C18 diacid [Octadecanedioic acid])
  source: /note=“See specification as filed for detailed description of substitutions and preferred embodiments”

SEQ ID NO: 97 - protein, 37 aa, Artificial Sequence
  Description of Artificial Sequence Synthetic polypeptide
  MOD_RES (8)..(8): AiB
  MOD_RES (9)..(9): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCXXGRLANFLVHSSNNFGAILPKTDVGANAP

SEQ ID NO: 98 - protein, 37 aa, Artificial Sequence; identical to SEQ ID NO: 97
  Description of Artificial Sequence Synthetic polypeptide
  MOD_RES (8)..(8): AiB
  MOD_RES (9)..(9): Lys-Ac-(glutamic acid linker)-(C18 diacid [Octadecanedioic acid])
  source: /note=“See specification as filed for detailed description of substitutions and preferred embodiments”

SEQ ID NO: 99 - protein, 37 aa, Artificial Sequence; identical to SEQ ID NO: 31
  Description of Artificial Sequence Synthetic polypeptide
  MOD_RES (8)..(8): AiB
  MOD_RES (11)..(11): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 100 - protein, 37 aa, Artificial Sequence; identical to SEQ ID NO: 31
  Description of Artificial Sequence Synthetic polypeptide
  MOD_RES (8)..(8): AiB
  MOD_RES (11)..(11): Lys-Ac-(glutamic acid linker)-(C18 diacid [Octadecanedioic acid])
  source: /note=“See specification as filed for detailed description of substitutions and preferred embodiments”

SEQ ID NO: 101 - protein, 38 aa, Artificial Sequence
  Description of Artificial Sequence Synthetic polypeptide
  MOD_RES (8)..(8): AiB
  MOD_RES (38)..(38): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCXLGRLANFLVHSSNNFGAILPKTDVGANAPX

SEQ ID NO: 102 - protein, 38 aa, Artificial Sequence; identical to SEQ ID NO: 101
  Description of Artificial Sequence Synthetic polypeptide
  MOD_RES (8)..(8): AiB
  MOD_RES (38)..(38): Lys-Ac-(glutamic acid linker)-(C18 diacid [Octadecanedioic acid])
  source: /note=“See specification as filed for detailed description of substitutions and preferred embodiments”

SEQ ID NO: 103 - protein, 32 aa, Artificial Sequence; identical to SEQ ID NO: 26
  Description of Artificial Sequence Synthetic polypeptide
  MOD_RES (11)..(11): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 104 - protein, 32 aa, Artificial Sequence
  Description of Artificial Sequence Synthetic polypeptide
  MOD_RES (9)..(9): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCVXGKLSQELHKLQTYPRTDVGANAP

SEQ ID NO: 105 - protein, 34 aa, Artificial Sequence
  Description of Artificial Sequence Synthetic polypeptide
  MOD_RES (34)..(34): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCVLGKLSQELHKLQTYPRTDVGANAPKX

SEQ ID NO: 106 - protein, 32 aa, Artificial Sequence; identical to SEQ ID NO: 83
  Description of Artificial Sequence Synthetic polypeptide
  MOD_RES (9)..(9): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 107 - protein, 32 aa, Artificial Sequence; identical to SEQ ID NO: 24
  Description of Artificial Sequence Synthetic polypeptide
  MOD_RES (11)..(11): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 108 - protein, 34 aa, Artificial Sequence; identical to SEQ ID NO: 84
  Description of Artificial Sequence Synthetic polypeptide
  MOD_RES (34)..(34): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 109 - protein, 32 aa, Artificial Sequence
  Description of Artificial Sequence Synthetic polypeptide
  MOD_RES (9)..(9): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CASLSTCVXGKLSQDLHKLQTFPKTDVGANAP

SEQ ID NO: 110 - protein, 32 aa, Artificial Sequence; identical to SEQ ID NO: 27
  Description of Artificial Sequence Synthetic polypeptide
  MOD_RES (11)..(11): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 111 - protein, 34 aa, Artificial Sequence
  Description of Artificial Sequence Synthetic polypeptide
  MOD_RES (34)..(34): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CASLSTCVLGKLSQDLHKLQTFPKTDVGANAPKX

SEQ ID NO: 112 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(2xOEG amino acids linked together with a glutamic acid residue attached to N-terminus)-(C20 diacid [Eicosanedioic acid])
  source: /note=“See specification as filed for detailed description of substitutions and preferred embodiments”

SEQ ID NO: 113 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(2xOEG amino acids linked together with a glutamic acid residue attached to N-terminus)-(C22 diacid [Docosanedioic acid])
  source: /note=“See specification as filed for detailed description of substitutions and preferred embodiments”

SEQ ID NO: 114 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(2xOEG amino acids linked together with a glutamic acid residue attached to N-terminus)-(C16 diacid [Hexadecanedioic acid])
  source: /note=“See specification as filed for detailed description of substitutions and preferred embodiments”

SEQ ID NO: 115 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(3xOEG amino acids linked together with a glutamic acid residue attached to N-terminus)-(C18 diacid [Octadecanedioic acid])
  source: /note=“See specification as filed for detailed description of substitutions and preferred embodiments”

SEQ ID NO: 116 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(1xOEG amino acids linked together with a glutamic acid residue attached to N-terminus)-(C18 diacid [Octadecanedioic acid])
  source: /note=“See specification as filed for detailed description of substitutions and preferred embodiments”

SEQ ID NO: 117 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(2xOEG amino acids linked together with a glutamic acid residue attached to N-terminus)-(C24 diacid [Tetracosanedioic acid])
  source: /note=“See specification as filed for detailed description of substitutions and preferred embodiments”

SEQ ID NO: 118 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(2xOEG amino acids linked together with a glutamic acid residue attached to N-terminus)-(C26 diacid [Hexacosanedioic acid])
  source: /note=“See specification as filed for detailed description of substitutions and preferred embodiments”

SEQ ID NO: 119 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(2xOEG amino acids linked together with a glutamic acid residue attached to N-terminus)-(C14 diacid [Tetradecanedioic acid])
  source: /note=“See specification as filed for detailed description of substitutions and preferred embodiments”

SEQ ID NO: 120 - protein, 32 aa, Artificial Sequence; identical to SEQ ID NO: 85
  Description of Artificial Sequence Synthetic polypeptide
  MOD_RES (8)..(8): AiB
  MOD_RES (9)..(9): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 121 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 122 - protein, 32 aa, Artificial Sequence
  Description of Artificial Sequence Synthetic polypeptide
  MOD_RES (8)..(8): AiB
  MOD_RES (12)..(12): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCXLGRXSQDLHRLQTYPKTDVGANAP

SEQ ID NO: 123 - protein, 32 aa, Artificial Sequence
  Description of Artificial Sequence Synthetic polypeptide
  MOD_RES (8)..(8): AiB
  MOD_RES (16)..(16): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCXLGRLSQDXHRLQTYPKTDVGANAP

SEQ ID NO: 124 - protein, 32 aa, Artificial Sequence
  Description of Artificial Sequence Synthetic polypeptide
  MOD_RES (8)..(8): AiB
  MOD_RES (18)..(18): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCXLGRLSQDLHXLQTYPKTDVGANAP

SEQ ID NO: 125 - protein, 32 aa, Artificial Sequence; identical to SEQ ID NO: 46
  Description of Artificial Sequence Synthetic polypeptide
  MOD_RES (8)..(8): AiB
  MOD_RES (19)..(19): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 126 - protein, 32 aa, Artificial Sequence; identical to SEQ ID NO: 46
  Description of Artificial Sequence Synthetic polypeptide
  MOD_RES (8)..(8): AiB
  MOD_RES (19)..(19): Lys-Ac-(2xOEG amino acids linked together with a glutamic acid residue attached to N-terminus)-(C22 diacid [Docosanedioic acid])
  source: /note=“See specification as filed for detailed description of substitutions and preferred embodiments”

SEQ ID NO: 127 - protein, 32 aa, Artificial Sequence
  Description of Artificial Sequence Synthetic polypeptide
  MOD_RES (8)..(8): AiB
  MOD_RES (24)..(24): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCXLGRLSQDLHRLQTYPXTDVGANAP

SEQ ID NO: 128 - protein, 34 aa, Artificial Sequence; identical to SEQ ID NO: 89
  Description of Artificial Sequence Synthetic polypeptide
  MOD_RES (8)..(8): AiB
  MOD_RES (34)..(34): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 129 - protein, 32 aa, Artificial Sequence
  Description of Artificial Sequence Synthetic polypeptide
  MOD_RES (9)..(9): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCMXGRLSQELHRLQTYPKTDVGANAP

SEQ ID NO: 130 - protein, 32 aa, Artificial Sequence; identical to SEQ ID NO: 25
  Description of Artificial Sequence Synthetic polypeptide
  MOD_RES (11)..(11): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 131 - protein, 32 aa, Artificial Sequence; identical to SEQ ID NO: 25
  Description of Artificial Sequence Synthetic polypeptide
  MOD_RES (11)..(11): Lys-Ac-(2xOEG amino acids linked together with a glutamic acid residue attached to N-terminus)-(C20 diacid [Eicosanedioic acid])
  source: /note=“See specification as filed for detailed description of substitutions and preferred embodiments”

SEQ ID NO: 132 - protein, 32 aa, Artificial Sequence; identical to SEQ ID NO: 25
  Description of Artificial Sequence Synthetic polypeptide
  MOD_RES (11)..(11): Lys-Ac-(2xOEG amino acids linked together with a glutamic acid residue attached to N-terminus)-(C22 diacid [Docosanedioic acid])
  source: /note=“See specification as filed for detailed description of substitutions and preferred embodiments”

SEQ ID NO: 133 - protein, 32 aa, Artificial Sequence
  Description of Artificial Sequence Synthetic polypeptide
  MOD_RES (12)..(12): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCMLGRXSQELHRLQTYPKTDVGANAP

SEQ ID NO: 134 - protein, 32 aa, Artificial Sequence
  Description of Artificial Sequence Synthetic polypeptide
  MOD_RES (16)..(16): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCMLGRLSQEXHRLQTYPKTDVGANAP

SEQ ID NO: 135 - protein, 32 aa, Artificial Sequence
  Description of Artificial Sequence Synthetic polypeptide
  MOD_RES (18)..(18): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCMLGRLSQELHXLQTYPKTDVGANAP

SEQ ID NO: 136 - protein, 32 aa, Artificial Sequence; identical to SEQ ID NO: 47
  Description of Artificial Sequence Synthetic polypeptide
  MOD_RES (19)..(19): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”

SEQ ID NO: 137 - protein, 32 aa, Artificial Sequence; identical to SEQ ID NO: 47
  Description of Artificial Sequence Synthetic polypeptide
  MOD_RES (19)..(19): Lys-Ac-(2xOEG amino acids linked together with a glutamic acid residue attached to N-terminus)-(C22 diacid [Docosanedioic acid])
  source: /note=“See specification as filed for detailed description of substitutions and preferred embodiments”

SEQ ID NO: 138 - protein, 32 aa, Artificial Sequence
  Description of Artificial Sequence Synthetic polypeptide
  MOD_RES (24)..(24): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCMLGRLSQELHRLQTYPXTDVGANAP

SEQ ID NO: 139 - protein, 34 aa, Artificial Sequence
  Description of Artificial Sequence Synthetic polypeptide
  MOD_RES (34)..(34): Lys-Ac-(2xOEG amino acids linked together with a glutamic acid residue attached to N-terminus)-(C18 diacid[Octadecanedioic acid])
  source: /note=“See specification as filed for detailed description of substitutions and preferred embodiments”
CSNLSTCMLGRLSQELHRLQTYPKTDVGANAPKX

SEQ ID NO: 140 - protein, 32 aa, Artificial Sequence; identical to SEQ ID NO: 24
  Description of Artificial Sequence Synthetic polypeptide
  MOD_RES (11)..(11): Lys-Ac
  source: /note=“See specification as filed for detailed description of substitutions and preferred embodiments”

SEQ ID NO: 141 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”

SEQ ID NO: 142 - protein, 32 aa, Artificial Sequence; identical to SEQ ID NO: 30
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”

SEQ ID NO: 143 - protein, 32 aa, Artificial Sequence; identical to SEQ ID NO: 26
  Description of Artificial Sequence Synthetic polypeptide
  MOD_RES (11)..(11): Lys-Ac
  source: /note=“See specification as filed for detailed description of substitutions and preferred embodiments”

SEQ ID NO: 144 - protein, 32 aa, Artificial Sequence; identical to SEQ ID NO: 25
  Description of Artificial Sequence Synthetic polypeptide
  MOD_RES (11)..(11): Lys-Ac
  source: /note=“See specification as filed for detailed description of substitutions and preferred embodiments”

SEQ ID NO: 145 - protein, 32 aa, Artificial Sequence; identical to SEQ ID NO: 27
  Description of Artificial Sequence Synthetic polypeptide
  MOD_RES (11)..(11): Lys-Ac
  source: /note=“See specification as filed for detailed description of substitutions and preferred embodiments”

SEQ ID NO: 146 - protein, 32 aa, Artificial Sequence
  Description of Artificial Sequence Synthetic polypeptide
  MOD_RES (11)..(11): Lys-Ac
  source: /note=“See specification as filed for detailed description of substitutions and preferred embodiments”
CASLSTCMLGXLSQDLHKLQTFPKTDVGANAP

SEQ ID NO: 147 - protein, 32 aa, Artificial Sequence; identical to SEQ ID NO: 28
  Description of Artificial Sequence Synthetic polypeptide
  MOD_RES (11)..(11): Lys-Ac
  source: /note=“See specification as filed for detailed description of substitutions and preferred embodiments”

SEQ ID NO: 148 - protein, 37 aa, Artificial Sequence; identical to SEQ ID NO: 31
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”

SEQ ID NO: 149 - protein, 33 aa, Artificial Sequence; identical to SEQ ID NO: 32
  Description of Artificial Sequence Synthetic polypeptide
  MOD_RES (11)..(11): Lys-Ac
  source: /note=“See specification as filed for detailed description of substitutions and preferred embodiments”

SEQ ID NO: 150 - protein, 35 aa, Artificial Sequence; identical to SEQ ID NO: 33
  Description of Artificial Sequence Synthetic polypeptide
  MOD_RES (11)..(11): Lys-Ac
  source: /note=“See specification as filed for detailed description of substitutions and preferred embodiments”

SEQ ID NO: 151 - protein, 36 aa, Artificial Sequence; identical to SEQ ID NO: 34
  Description of Artificial Sequence Synthetic polypeptide
  MOD_RES (11)..(11): Lys-Ac
  source: /note=“See specification as filed for detailed description of substitutions and preferred embodiments”

SEQ ID NO: 152 - protein, 37 aa, Artificial Sequence; identical to SEQ ID NO: 35
  Description of Artificial Sequence Synthetic polypeptide
  MOD_RES (11)..(11): Lys-Ac
  source: /note=“See specification as filed for detailed description of substitutions and preferred embodiments”

SEQ ID NO: 153 - protein, 37 aa, Artificial Sequence; identical to SEQ ID NO: 36
  Description of Artificial Sequence Synthetic polypeptide
  MOD_RES (11)..(11): Lys-Ac
  source: /note=“See specification as filed for detailed description of substitutions and preferred embodiments”

SEQ ID NO: 154 - protein, 37 aa, Artificial Sequence; identical to SEQ ID NO: 37
  Description of Artificial Sequence Synthetic polypeptide
  MOD_RES (11)..(11): Lys-Ac
  source: /note=“See specification as filed for detailed description of substitutions and preferred embodiments”

SEQ ID NO: 155 - protein, 36 aa, Artificial Sequence; identical to SEQ ID NO: 38
  Description of Artificial Sequence Synthetic polypeptide
  MOD_RES (11)..(11): Lys-Ac
  source: /note=“See specification as filed for detailed description of substitutions and preferred embodiments”

SEQ ID NO: 156 - protein, 36 aa, Artificial Sequence; identical to SEQ ID NO: 39
  Description of Artificial Sequence Synthetic polypeptide
  MOD_RES (11)..(11): Lys-Ac
  source: /note=“See specification as filed for detailed description of substitutions and preferred embodiments”

SEQ ID NO: 157 - protein, 36 aa, Artificial Sequence; identical to SEQ ID NO: 40
  Description of Artificial Sequence Synthetic polypeptide
  MOD_RES (11)..(11): Lys-Ac
  source: /note=“See specification as filed for detailed description of substitutions and preferred embodiments”

SEQ ID NO: 158 - protein, 36 aa, Artificial Sequence; identical to SEQ ID NO: 41
  Description of Artificial Sequence Synthetic polypeptide
  MOD_RES (11)..(11): Lys-Ac
  source: /note=“See specification as filed for detailed description of substitutions and preferred embodiments”

SEQ ID NO: 159 - protein, 36 aa, Artificial Sequence; identical to SEQ ID NO: 42
  Description of Artificial Sequence Synthetic polypeptide
  MOD_RES (11)..(11): Lys-Ac
  source: /note=“See specification as filed for detailed description of substitutions and preferred embodiments”

SEQ ID NO: 160 - protein, 36 aa, Artificial Sequence; identical to SEQ ID NO: 43
  Description of Artificial Sequence Synthetic polypeptide
  MOD_RES (11)..(11): Lys-Ac
  source: /note=“See specification as filed for detailed description of substitutions and preferred embodiments”

SEQ ID NO: 161 - protein, 36 aa, Artificial Sequence; identical to SEQ ID NO: 44
  Description of Artificial Sequence Synthetic polypeptide
  MOD_RES (11)..(11): Lys-Ac
  source: /note=“See specification as filed for detailed description of substitutions and preferred embodiments”

SEQ ID NO: 162 - protein, 36 aa, Artificial Sequence; identical to SEQ ID NO: 45
  Description of Artificial Sequence Synthetic polypeptide
  MOD_RES (11)..(11): Lys-Ac
  source: /note=“See specification as filed for detailed description of substitutions and preferred embodiments”

SEQ ID NO: 163 - protein, 32 aa, Artificial Sequence; identical to SEQ ID NO: 46
  Description of Artificial Sequence Synthetic polypeptide
  MOD_RES (8)..(8): AiB
  MOD_RES (19)..(19): Lys-Ac
  source: /note=“See specification as filed for detailed description of substitutions and preferred embodiments”

SEQ ID NO: 164 - protein, 32 aa, Artificial Sequence; identical to SEQ ID NO: 47
  Description of Artificial Sequence Synthetic polypeptide
  MOD_RES (19)..(19): Lys-Ac
  source: /note=“See specification as filed for detailed description of substitutions and preferred embodiments”

SEQ ID NO: 165 - protein, 32 aa, Artificial Sequence; identical to SEQ ID NO: 24
  Description of Artificial Sequence Synthetic polypeptide
  MOD_RES (11)..(11): Lys-Ac
  source: /note=“See specification as filed for detailed description of substitutions and preferred embodiments”

SEQ ID NO: 166 - protein, 32 aa, Artificial Sequence; identical to SEQ ID NO: 29
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”

SEQ ID NO: 167 - protein, 32 aa, Artificial Sequence; identical to SEQ ID NO: 30
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”

SEQ ID NO: 168 - protein, 32 aa, Artificial Sequence; identical to SEQ ID NO: 26
  Description of Artificial Sequence Synthetic polypeptide
  MOD_RES (11)..(11): Lys-Ac
  source: /note=“See specification as filed for detailed description of substitutions and preferred embodiments”

SEQ ID NO: 169 - protein, 32 aa, Artificial Sequence; identical to SEQ ID NO: 25
  Description of Artificial Sequence Synthetic polypeptide
  MOD_RES (11)..(11): Lys-Ac
  source: /note=“See specification as filed for detailed description of substitutions and preferred embodiments”

SEQ ID NO: 170 - protein, 32 aa, Artificial Sequence; identical to SEQ ID NO: 27
  Description of Artificial Sequence Synthetic polypeptide
  MOD_RES (11)..(11): Lys-Ac
  source: /note=“See specification as filed for detailed description of substitutions and preferred embodiments”

SEQ ID NO: 171 - protein, 32 aa, Artificial Sequence; identical to SEQ ID NO: 146
  Description of Artificial Sequence Synthetic polypeptide
  MOD_RES (11)..(11): Lys-Ac
  source: /note=“See specification as filed for detailed description of substitutions and preferred embodiments”

SEQ ID NO: 172 - protein, 32 aa, Artificial Sequence; identical to SEQ ID NO: 28
  Description of Artificial Sequence Synthetic polypeptide
  MOD_RES (11)..(11): Lys-Ac
  source: /note=“See specification as filed for detailed description of substitutions and preferred embodiments”

SEQ ID NO: 173 - protein, 37 aa, Artificial Sequence; identical to SEQ ID NO: 31
  Description of Artificial Sequence Synthetic polypeptide
  MOD_RES (8)..(8): AiB
  MOD_RES (11)..(11): Lys-Ac
  source: /note=“See specification as filed for detailed description of substitutions and preferred embodiments”

SEQ ID NO: 174 - protein, 33 aa, Artificial Sequence; identical to SEQ ID NO: 32
  Description of Artificial Sequence Synthetic polypeptide
  MOD_RES (11)..(11): Lys-Ac
  source: /note=“See specification as filed for detailed description of substitutions and preferred embodiments”

SEQ ID NO: 175 - protein, 35 aa, Artificial Sequence; identical to SEQ ID NO: 33
  Description of Artificial Sequence Synthetic polypeptide
  MOD_RES (11)..(11): Lys-Ac
  source: /note=“See specification as filed for detailed description of substitutions and preferred embodiments”

SEQ ID NO: 176 - protein, 36 aa, Artificial Sequence; identical to SEQ ID NO: 34
  Description of Artificial Sequence Synthetic polypeptide
  MOD_RES (11)..(11): Lys-Ac
  source: /note=“See specification as filed for detailed description of substitutions and preferred embodiments”

SEQ ID NO: 177 - protein, 37 aa, Artificial Sequence; identical to SEQ ID NO: 35
  Description of Artificial Sequence Synthetic polypeptide
  MOD_RES (11)..(11): Lys-Ac
  source: /note=“See specification as filed for detailed description of substitutions and preferred embodiments”

SEQ ID NO: 178 - protein, 37 aa, Artificial Sequence; identical to SEQ ID NO: 36
  Description of Artificial Sequence Synthetic polypeptide
  MOD_RES (11)..(11): Lys-Ac
  source: /note=“See specification as filed for detailed description of substitutions and preferred embodiments”

SEQ ID NO: 179 - protein, 37 aa, Artificial Sequence; identical to SEQ ID NO: 37
  Description of Artificial Sequence Synthetic polypeptide
  MOD_RES (11)..(11): Lys-Ac
  source: /note=“See specification as filed for detailed description of substitutions and preferred embodiments”

SEQ ID NO: 180 - protein, 36 aa, Artificial Sequence; identical to SEQ ID NO: 38
  Description of Artificial Sequence Synthetic polypeptide
  MOD_RES (11)..(11): Lys-Ac
  source: /note=“See specification as filed for detailed description of substitutions and preferred embodiments”

SEQ ID NO: 181 - protein, 36 aa, Artificial Sequence; identical to SEQ ID NO: 39
  Description of Artificial Sequence Synthetic polypeptide
  MOD_RES (11)..(11): Lys-Ac
  source: /note=“See specification as filed for detailed description of substitutions and preferred embodiments”

SEQ ID NO: 182 - protein, 36 aa, Artificial Sequence; identical to SEQ ID NO: 40
  Description of Artificial Sequence Synthetic polypeptide
  MOD_RES (11)..(11): Lys-Ac
  source: /note=“See specification as filed for detailed description of substitutions and preferred embodiments”

SEQ ID NO: 183 - protein, 36 aa, Artificial Sequence; identical to SEQ ID NO: 41
  Description of Artificial Sequence Synthetic polypeptide
  MOD_RES (11)..(11): Lys-Ac
  source: /note=“See specification as filed for detailed description of substitutions and preferred embodiments”

SEQ ID NO: 184 - protein, 36 aa, Artificial Sequence; identical to SEQ ID NO: 42
  Description of Artificial Sequence Synthetic polypeptide
  MOD_RES (11)..(11): Lys-Ac
  source: /note=“See specification as filed for detailed description of substitutions and preferred embodiments”

SEQ ID NO: 185 - protein, 36 aa, Artificial Sequence; identical to SEQ ID NO: 43
  Description of Artificial Sequence Synthetic polypeptide
  MOD_RES (11)..(11): Lys-Ac
  source: /note=“See specification as filed for detailed description of substitutions and preferred embodiments”

SEQ ID NO: 186 - protein, 36 aa, Artificial Sequence; identical to SEQ ID NO: 44
  Description of Artificial Sequence Synthetic polypeptide
  MOD_RES (11)..(11): Lys-Ac
  source: /note=“See specification as filed for detailed description of substitutions and preferred embodiments”

SEQ ID NO: 187 - protein, 36 aa, Artificial Sequence; identical to SEQ ID NO: 45
  Description of Artificial Sequence Synthetic polypeptide
  MOD_RES (11)..(11): Lys-Ac
  source: /note=“See specification as filed for detailed description of substitutions and preferred embodiments”

SEQ ID NO: 188 - protein, 32 aa, Artificial Sequence; identical to SEQ ID NO: 46
  Description of Artificial Sequence Synthetic polypeptide
  MOD_RES (8)..(8): AiB
  MOD_RES (19)..(19): Lys-Ac
  source: /note=“See specification as filed for detailed description of substitutions and preferred embodiments”

SEQ ID NO: 189 - protein, 32 aa, Artificial Sequence; identical to SEQ ID NO: 25
  Description of Artificial Sequence Synthetic polypeptide
  MOD_RES (11)..(11): Lys-Ac
  source: /note=“See specification as filed for detailed description of substitutions and preferred embodiments”